(19) United States
(12) Patent Application Publication
(10) Pub. No.: US 2011/0318728 A1
(43) Pub. Date: Dec. 29, 2011

(54) SYSTEMS, DEVICES, METHODS AND KITS FOR FLUID HANDLING

(76) Inventors: Huan Lac Phan, Belmont, CA (US); Stanley Paul Woods, Cupertino, CA (US)

(21) Appl. No.: 13/140,996

(22) PCT Filed: Dec. 30, 2009
(86) PCT No.: PCT/US09/69812

§ 371 Date: Jul. 26, 2011

Related U.S. Application Data
(60) Provisional application No. 61/166,760, filed on Apr. 5, 2009.
(60) Provisional application No. 61/141,645, filed on Dec. 30, 2008.

(51) Int. Cl. C12Q 1/70; C12Q 1/68; B01L 3/00; C12M 1/33; B01L 99/00; G01N 1/34; C12M 1/34

(52) U.S. Cl. 435/5; 436/174; 435/6.1; 435/6.15; 435/287.2; 436/17; 435/306.1; 422/501; 422/513; 422/68.1; 422/547

(57) ABSTRACT

Fluid handling devices, systems, methods and kits are disclosed. Fluid handling devices according to the disclosure comprise: an inlet for receiving a sample; a reagent layer comprising, a substrate having a first surface and an opposing second surface, at least one reagent storage compartment configured to hold a reagent, and a seal in communication with the at least one reagent storage compartment; and a reaction layer having a first surface and an opposing second surface comprising, a reaction area, and an outlet in communication with the reaction area, wherein the reagent layer and reaction layer are adapted and configured to permit movement of at least one of the reagent layer and the reaction layer in a plane relative to the other layer.

### CROSS-REFERENCE

This application claims the benefit of U.S. Provisional Application No. 61/141,645, filed Dec. 30, 2008, and Application No. 61/166,760, filed Apr. 5, 2009, which applications are incorporated herein by reference.

### BACKGROUND OF THE INVENTION

Molecular Diagnostics (MDx) is the fastest growing segment of the In vitro Diagnostic (IVD) market and is projected to be on the order of $10 billion dollars by 2015. These tests are typically more sensitive, more specific and more timely than earlier generation tests and/or provide information that is unavailable with any other approaches. Examples of MDx applications include assessment of a patient's predisposition to diseases, determination of their likely response to therapeutics, and identification of infectious agents either in the standard clinical settings or in the context of bioterrorism or biodefense.

To date, only several hundreds of the approximately 60,000 CLIA (Clinical Laboratory Improvement Amendment) certified laboratories are capable of performing MDx tests. This is a reflection of the complex workflows of current MDx tests and their requirement for highly trained staff in a controlled laboratory environment to execute these tests. The market for MDx is likely to expand outside of the existing CLIA certified laboratories as demands for MDx rises from the emerging retail health clinics and point of care testing markets.

Despite these exploding demands for MDx, the ability to support these tests is declining under the current system. It is estimated that the number of available trained CLIA staff will decrease by 60-70% within the next few years due to the prevailing demographic shift (retirements) and the lack of trained replacement personnel. These trends point to the need to reduce the complexity of these tests to enable their use by more naïve staff, to disconnect them from the traditional clinical labs and make them available in distributed locations closer to the patient.

Diagnostic assays frequently require sample preparation steps in order to remove substances that interfere with the assays and/or to increase the concentration of analytes in the sample. These sample preparation steps for biological samples often include pre-treatment of the sample to disrupt or lyse the cellular materials (for example by mechanical or enzymatic treatments) to release analytes into solution. These solutions, or lysates, are introduced to a reaction area which specifically binds analytes of interest. These reaction areas are then washed to further remove contaminants. Finally, the analytes are eluted from the reaction area for downstream detection by various analytical methods sensitive to the analytes. This “bind, wash, elute” process is a dominant paradigm in sample processing for MDx.

Nucleic acids are a common class of analytes targeted by MDx and must be released from the cellular bodies so that they become accessible for analysis. By way of background and to provide context for the invention, FIG. 1 illustrates a commonly used process for the analysis of nucleic acids from cellular materials as currently practiced. For simple targets such as some viruses, release of the nucleic acids from cellular bodies is easily accomplished by mixing the viral sample with a lysis buffer. For other targets with sturdier cell walls, enzymatic incubation or mechanical means (e.g. bead beating, sonication) are needed to breach these cell walls and release the nucleic acids into the lysate. Other suitable means to breach these cell walls can be used without departing from the scope of the invention. When enzymatic incubation is used, typically, the samples are mixed with a dedicated lysis buffer optimized for enzymatic activity.

There are numerous solid phases which can function as a reaction area and which are adapted to bind nucleic acids, some examples are: glass or silica based surfaces (U.S. Pat. Nos. 5,234,809, 6,787,307), carboxylated surfaces (U.S. Pat. No. 5,705,628), or pH sensitive surfaces (U.S. Pat. No. 6,914,137). The lysates are typically combined with a binding buffer prior to their introduction to the solid surface. The binding buffer promotes selective binding of the nucleic acids to the solid phase over other undesirable substances. The exact composition of the binding buffer varies greatly depending on the solid phase that is used, but in the case of silica based materials, a strong chaotrope such as guanidine is usually a major constituent of the binding buffer. Use of chaotrope based binding buffers have advantages in that they can serve as lysis buffer for many sample types, and they also act as a preservative for nucleic acids. Nevertheless, many successful purification processes do not use the binding buffer as the lysis buffer and a dedicated binding buffer must be added to the lysate. Even when a chaotrope based binding/lysis buffer is used, it can be advantageous, as described in U.S. Pat. No. 6,383,393, to add a second binding buffer containing alcohols prior to solid phase binding. Use of a second binding buffer can improve the binding efficiency of nucleic acids to the solid phase but adds an additional fluid transfer step to the overall process.

As FIG. 1 shows, once the analyte(s) have been bound to the solid phase, the remaining fluids are emptied to waste and the solid phase is washed with one or more wash buffers to remove any remaining contaminants. These washes are also emptied to waste. The remaining bound analyte(s) are eluted from the solid phase upon introduction of an elution buffer and the eluate is transferred to a collection vessel for downstream analysis/detection. While the exact process can vary depending on the sample type, application and solid phase that are used, the arrows connecting each of the process boxes in FIG. 1 can be viewed as a fluid transfer step and/or manipulation of test tubes or solid phases that must be performed by a trained laboratory specialist (as denoted by the lab figure positioned next to each arrow where a manual step is performed or a step that requires user interaction). These steps are numerous, tedious, and prone to errors. Moreover, the repetitive steps involved increase the likelihood that workers will suffer from repetitive stress injuries such as carpal tunnel.

Since their inception, improvements have been made to the solid phase form factor to improve their utility. Current commercially available solid phases typically come in the form of filtration columns or as coated magnetic beads. The QIAGEN line of QlAprep® (Hilden, Germany) products is an example of a spin filter based form factor. Fluids are introduced to the filter by pipetting into the reservoir on top of the filter unit. Centrifugal force is used to drive the fluids through the filter. The lysate and wash are filtered into waste collection tubes, while the elution buffer is filtered into a dedicated collection tube. The Fuji QuickGene®-Mini80 (Tokyo, Japan) is a similar product which uses pressure as the driving force to move fluids through the filter.

An alternative solid phase form factor is the coated magnetic bead. Here the fluids are mixed in a container with the beads and the two are allowed to interact. Fluid exchange is accomplished by placing an external magnet next to the container, attracting the beads to the container wall, removing the old fluid and replacing it with the new fluid of choice. At the end of the process, the eluate is transferred to a separate dedicated collection tube. One potential drawback of the bead based process is that processing of large volumes of lysate requires larger numbers of beads (which can increase cost) and longer times to allow for beads/fluid interaction and subsequent attraction by the external magnet to the wall (which slows the overall process).

When these solid phase form factors were introduced, the fluid transfer and manipulation steps were performed manually. Recently, more automated and integrated systems have been introduced. Examples of more automated systems which employ filter based technologies include the QIAGEN QIACube®, the Fuji QuickGene®-800, and the Cepheid GeneXpert® (Sunnyvale, Calif.). Systems which uses bead based solid phases include the Promega Maxwell®-16 (Madison, Wis.), Roche MagNA Pure® (Indianapolis, Ind.), and Iquum Liat™ (Marlborough, Mass.) Analyzer. All of these systems are able to automate the fluid handling steps of MDx sample preparation to varying levels of success. But this is but the first hurdle to provide MDx access to the insufficiently staffed CLIA labs, the Point of Care (POC) practitioners, or retail health clinics. A compelling set of features important to access these markets include: 1) Simple to use consumable with on-board reagents. Systems with reagent bottles and tubings present challenges to apparatus maintenance and reagent tracking. 2) Inexpensive consumable. 3) Ability to carryout the lysis step in addition to the “bind, wash, elute” sample preparation steps. 4) Effective waste containment for enhanced safety. 5) Flexible enough to employ single or dual binding buffer chemistry. 6) Able to effectively handle large input sample volume. 7) Can process single sample at a time, but configurable to handle multiple samples when needed. This single sample capability is important in the clinical setting for urgent processing of “STAT” samples. 8) Easily adaptable for introduction of the eluate into any downstream consumable for analytical detection. 9) Ease of integration of sample preparation with downstream detection into a single apparatus.

While the existing systems described above may each contain some combinations of these features, none have all of these important features.

### SUMMARY OF THE INVENTION

An aspect of the disclosure is directed to fluid handling devices. Fluid handling devices comprise: an inlet for receiving a sample; a reagent layer comprising, a substrate having a first surface and an opposing second surface, at least one reagent storage compartment configured to hold a reagent, and a seal in communication with the at least one reagent storage compartment; and a reaction layer having a first surface and an opposing second surface comprising, a reaction area, and an outlet in communication with the reaction area, wherein the reagent layer and reaction layer are adapted and configured to permit movement of at least one of the reagent layer and the reaction layer in a plane relative to the other layer. Moreover, one or more of a reagent layer support extending from the substrate or a reaction layer support can be provided. Additionally, one or more of each of a shoulder, a seal, such as a selectively openable seal, and one or more reagent storage compartments, which may be compressible, may also be provided. The reagent layer may further be configured to comprise two or more reagent storage compartments and further wherein at least two of the reagent storage compartments of the reagent layer are in fluid communication. Where two or more reagent compartments are provided, two or more reagents may be provided as well. The reagent storage compartment may further be configured to comprise a tip. The tip can be configured such that it enables at least one of mixing of material within the reaction area, transfer of material from a first reaction area to a second reaction area, and transfer of material from a reaction area to a fluid collection compartment. Additionally, the tip may further comprises a selectively openable seal. In some configurations, the reaction area may further be configured to comprise one or more filters. Additionally, one or more reaction vessels may be provided, each of which also may include a reagent. In other aspects a fluid collection compartment may be provided. Additional external positioning features may be provided that are adapted and configured to engage the reagent layer with the reaction layer. A fluid collection layer may be provided, which may, for example also be configured to comprise one or more inlets, provide one or more materials adapted and configured to absorb waste, and comprise one or more fluid collection compartments, which can also contain a reagent and can be sealable. Additionally, at least one of the reagent layer and the reaction layer is nestable within the other layer and the reagent compartment of the reagent layer may also be adaptable to form a pressure tight seal in some configurations. The reagent layer and reaction layer can further be adapted and configured to move at least one of about an axis and along and axis. Additionally, at least one of the reagent layer and the reaction layer are adapted and configured to permit movement in a plane relative to the fluid collection layer. In some configurations, the reagent layer and reaction layer are integrally formed. Moreover, one or more external positioning features can be provided that are adapted and configured to engage the reaction layer with the fluid collection layer. The external positioning features can further be adapted and configured to prevent movement of at least one of the reagent layer and the reaction layer relative to the other layer. Additionally, the external positioning features are adapted and configured to at least one of prevent movement of at least one of the reagent layer, the reaction layer and the fluid collection relative to at least one other layer and permit movement of at least one of the reagent layer, the reaction layer and the fluid collection relative to at least one other layer. The reaction layer may also be in fluid communication with a first reagent storage compartment at a first time and a second reagent storage compartment at a second time, e.g. by a channel or a tube or some other mechanism that permits fluid from one storage compartment to access another storage compartment. Any configuration of components or devices can further comprise one or more device identification components. The one or more device identification components are adapted and configured to identify one or more of each of serial number, manufacturer, lot number, date codes, reagent type, reagent volume, reaction area type, process identification, process parameters needed to run the process, and calibration parameter. Additionally, the one or more device identification components may also be associated with at least one or more of the fluid handling device, the reagent layer, the reaction layer, and the fluid collection compartment. Moreover, the one or more device identification components associated with at least one or more of the fluid handling device, the reagent layer, the reaction layer, and the fluid collection compartment are adapted and configured to communicate information between the one or more of the fluid handling device, the reagent layer, the reaction layer, and the fluid collection compartment. The diagnostic device and/or an adapter between the diagnostic device and a fluid handling device can further be adapted and configured to activate the fluid handling device such that the fluid handling device begins processing a sample.

Another aspect of the disclosure is directed to systems adapted and configured to process fluid. Fluid processing systems comprise: a diagnostic device; and a fluid handling device comprising, an inlet for receiving a sample, a reagent layer comprising, a substrate having a first surface and an opposing second surface, at least one reagent storage compartment configured to hold a reagent, a seal in communication with the at least one reagent storage compartment, a reaction layer having a first surface and an opposing second surface comprising, a reaction area, and an outlet in communication with the reaction area, wherein the reagent layer and reaction layer are adapted and configured to permit movement of at least one of the reagent layer and the reaction layer in a plane relative to the other layer. Moreover, one or more of a reagent layer support extending from the substrate or a reaction layer support can be provided. Additionally, one or more of each of a shoulder, a seal, such as a selectively openable seal, and one or more reagent storage compartments, which may be compressible, may also be provided. The reagent layer may further be configured to comprise two or more reagent storage compartments and further wherein at least two of the reagent storage compartments of the reagent layer are in fluid communication. Where two or more reagent compartments are provided, two or more reagents may be provided as well. The reagent storage compartment may further be configured to comprise a tip. The tip can be configured such that it enables at least one of mixing of material within the reaction area, transfer of material from a first reaction area to a second reaction area, and transfer of material from a reaction area to a fluid collection compartment. Additionally, the tip may further comprises a selectively openable seal. In some configurations, the reaction area may further be configured to comprise one or more filters. Additionally, one or more reaction vessels may be provided, each of which also may include a reagent. In other aspects a fluid collection compartment may be provided. Additional external positioning features may be provided that are adapted and configured to engage the reagent layer with the reaction layer. A fluid collection layer may be provided, which may, for example also be configured to comprise one or more inlets, provide one or more materials adapted and configured to absorb waste, and comprise one or more fluid collection compartments, which can also contain a reagent and can be sealable. Additionally, at least one of the reagent layer and the reaction layer is nestable within the other layer and the reagent compartment of the reagent layer may also be adaptable to form a pressure tight seal in some configurations. The reagent layer and reaction layer can further be adapted and configured to move at least one of about an axis and along and axis. Additionally, at least one of the reagent layer and the reaction layer are adapted and configured to permit movement in a plane relative to the fluid collection layer. In some configurations, the reagent layer and reaction layer are integrally formed. Moreover, one or more external positioning features can be provided that are adapted and configured to engage the reaction layer with the fluid collection layer. The external positioning features can further be adapted and configured to prevent movement of at least one of the reagent layer and the reaction layer relative to the other layer. Additionally, the external positioning features are adapted and configured to at least one of prevent movement of at least one of the reagent layer, the reaction layer and the fluid collection relative to at least one other layer and permit movement of at least one of the reagent layer, the reaction layer and the fluid collection relative to at least one other layer. The reaction layer may also be in fluid communication with a first reagent storage compartment at a first time and a second reagent storage compartment at a second time, e.g. by a channel or a tube or some other mechanism that permits fluid from one storage compartment to access another storage compartment. Any configuration of components or devices can further comprise one or more device identification components. The one or more device identification components are adapted and configured to identify one or more of each of serial number, manufacturer, lot number, date codes, reagent type, reagent volume, reaction area type, process identification, process parameters needed to run the process, and calibration parameter. Additionally, the one or more device identification components may also be associated with at least one or more of the fluid handling device, the reagent layer, the reaction layer, and the fluid collection compartment. Moreover, the one or more device identification components associated with at least one or more of the fluid handling device, the reagent layer, the reaction layer, and the fluid collection compartment are adapted and configured to communicate information between the one or more of the fluid handling device, the reagent layer, the reaction layer, and the fluid collection compartment. The diagnostic device and/or an adapter between the diagnostic device and a fluid handling device can further be adapted and configured to activate the fluid handling device such that the fluid handling device begins processing a sample.

Still another aspect of the disclosure is directed to methods for processing a sample. Methods include, for example, obtaining a sample; inserting a sample into a reagent layer further comprising, a substrate having a first surface and an opposing second surface, one or more reagent storage compartments configured to hold a reagent, and one or more seals enclosing the one or more reagent storage compartments, reacting the sample in a reaction layer having a first surface and an opposing second surface comprising, a reaction area, and an outlet in communication with the reaction area, wherein the reagent layer and reaction layer are adapted and configured to permit movement of at least one of the reagent layer and the reaction layer in a plane relative to the other layer; and processing the sample without human interaction with the sample after the step of inserting the sample into the reagent layer. Additional steps of the method can include, for example, one or more of each of the following steps of: delivering at least one processed sample to the diagnostic machine, analyzing the at least one processed sample, one or more of adding a lysis buffer to the sample, adding a binding buffer to the sample, binding the sample to a reaction area, emptying a fluid into a waste container; adding a wash buffer; adding an elution buffer; and eluting the sample, controlling at least one of a temperature, a reaction time, and a motion, analyzing the processed sample, and delivering at least two processed samples to the diagnostic machine. Samples include, biological samples, which includes, but are not limited to, nucleic acids, blood, nasal washes, suspensions of particulates (such as dirt or feces), other cellular suspensions (such as saliva, cheek swabs, scabs, nail clippings, hair, buccal swabs), protein suspensions, mixtures of compounds and the like. Suitable diagnostic devices for use with the method include, for examples, molecular diagnostic devices, polymerase chain reaction devices, isothermal amplification devices, lateral flow devices, devices employing arrays, electrochemical detection devices, optical detection devices, nucleic acid sequencers. The fluid handling device can further be activated such that the fluid handling device begins processing a sample. Activation can be via a network command, an adapter configured to communication between a diagnostic device and the fluid handling device or via the diagnostic device itself.

Yet another aspect of the disclosure is directed to kits for processing a sample. Kits include, for example, a reagent layer comprising, a substrate having a first surface and an opposing second surface, at least one reagent storage compartment configured to contain a reagent, and a seal in communication with the at least one reagent storage compartment; a packaging adapted and configured to house one or more kit components. Another kit could include, for example, a reaction layer comprising a first surface and an opposing second surface, comprising a reaction area, and an outlet in communication with the reaction area; a packaging adapted and configured to house one or more kit components. Additional components of any kit could include one or more of each of filters, a reaction layer having a first surface and an opposing second surface, comprising a reaction area, and an outlet in communication with the reaction area and a fluid collection compartment, reagents, syringes adapted and configured to deliver the reagents to the reagent layer, fluid collection layers, reaction vessels, reaction area columns, eluate collection vessels, adapters to engage a diagnostic device and a fluid handling device or components of a fluid handling device, detectors, device identification components. Suitable reagents include one or more of the following lysis buffers, binding buffers, wash buffers, elution buffers, reaction buffers, dilution buffers, aqueous solutions, organic solutions, protein solutions, and dried reagents.

Additional aspects of the disclosure relate to a communication system. The communication system comprises: a diagnostic device; a fluid handling device comprising an inlet for receiving a sample, a reagent layer comprising a substrate having a first surface and an opposing second surface, at least one reagent storage compartment configured to hold a reagent, and a seal in communication with the at least one reagent storage compartment, and a reaction layer having a first surface and an opposing second surface comprising, a reaction area, and an outlet in communication with the reaction area, wherein the reagent layer and the reaction layer are adapted and configured to permit movement of at least one of the reagent layer and the reaction layer in a plane relative to the other layer; a diagnostic device server computer system; a diagnostic test result module on the server computer system for permitting the transmission of a diagnostic test result from a diagnostic device over a network; at least one of an API engine connected to at least one of the diagnostic device and the fluid handling device to create a message about the diagnostic test result and transmit the message over an API integrated network to a recipient having a predetermined recipient user name, an SMS engine connected to at least one of the diagnostic device and the fluid handling device to create an SMS message about the diagnostic test result and transmit the SMS message over a network to a recipient device having a predetermined diagnostic test result recipient telephone number, and an email engine connected to at least one of the diagnostic device and the fluid handling device to create an email message about the diagnostic test result and transmit the email message over the network to a diagnostic test result recipient email having a predetermined diagnostic test result recipient email address. Additionally, a storing module can be provided on the server computer system for storing the diagnostic test result on the diagnostic device server database. In some configurations, at least one of the diagnostic device and the fluid handling device is connectable to the server computer system over at least one of a mobile phone network and an Internet network, and a browser on the diagnostic test result recipient electronic device is used to retrieve an interface on the server computer system. The system can be configured such that a plurality of email addresses are held in a diagnostic device database (e.g., email addresses of physicians requesting tests, patients for whom tests are performed, law enforcement personnel, etc.) and fewer than all the email addresses are individually selectable from the diagnostic host computer system (e.g., only the email addresses which should receive a particular test result), the email message being transmitted to at least one diagnostic test result recipient email having at least one selected email address. At least one of the diagnostic device and the fluid handling device can also be connectable to the server computer system over the Internet, and a browser on the diagnostic test result recipient electronic device is used to retrieve an interface on the server computer system. Additionally, a plurality of user names are held in the diagnostic device database and fewer than all the user names are individually selectable from the diagnostic host computer system, the message being transmitted to at least one diagnostic test result recipient user name via an API. In other configurations, the diagnostic test result recipient electronic device is connected to the server computer system over a cellular phone network, for example, such that it is in communication with a mobile device. An interface on the server computer system can be provided such that the interface is retrievable by an application on the diagnostic test result recipient mobile device. In that case, the SMS diagnostic test result can then be received by a message application on the diagnostic test result recipient mobile device. Moreover, a plurality of SMS diagnostic test results are received for the diagnostic test result, each by a respective message application on a respective diagnostic test result recipient mobile device. Additionally, at least one SMS engine can be configured to receive an SMS response over the cellular phone SMS network from the diagnostic test result recipient mobile device and stores an SMS response on the server computer system. In some situations, a diagnostic test result recipient phone number ID can be transmitted with the SMS diagnostic test result to the SMS engine and is used by the server computer system to associate the SMS diagnostic test result with the SMS response. The server computer system can also be connectable over a cellular phone network to receive a response from the diagnostic test result recipient mobile device. Additionally, in some configurations, the SMS diagnostic test result includes a URL that is selectable at the diagnostic test result recipient mobile device to respond from the diagnostic test result recipient mobile device to the server computer system, the server computer system utilizing the URL to associate the response with the SMS diagnostic test result. The communications system can also comprise a downloadable application residing on the diagnostic test result recipient mobile device, the downloadable application transmitting the response and a diagnostic test result recipient phone number ID over the cellular phone network to the server computer system, the server computer system utilizing the diagnostic test result recipient phone number ID to associate the response with the SMS diagnostic test result, a transmissions module that transmits the diagnostic test result over a network other than the cellular phone SMS network to a diagnostic test result recipient user computer system, in parallel with the diagnostic test result that is sent over the cellular phone SMS network, and/or a downloadable application residing on the diagnostic test result recipient host computer, the downloadable application transmitting a response and a diagnostic test result recipient phone number ID over the cellular phone network to the server computer system, the server computer system utilizing the diagnostic test result recipient phone number ID to associate the response with the SMS diagnostic test result. The communication system can further be adapted to activate the fluid handling device such that the fluid handling device begins processing a sample.

Another aspect of the disclosure is directed to a networked apparatus or group of apparatuses. The network apparatus comprises: a memory; a processor; a communicator; a display; a fluid handling device comprising an inlet for receiving a sample, a reagent layer comprising a substrate having a first surface and an opposing second surface, at least one reagent storage compartment configured to hold a reagent, and a seal in communication with the at least one reagent storage compartment, and a reaction layer having a first surface and an opposing second surface comprising, a reaction area, and an outlet in communication with the reaction area, wherein the reagent layer and the reaction layer are adapted and configured to permit movement of at least one of the reagent layer and the reaction layer in a plane relative to the other layer. The networked apparatuses can further be adapted to enable activation of the fluid handling device via the network such that the fluid handling device begins processing a sample.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates a commonly used process for the analysis of nucleic acids from cellular materials;

FIG. 2A illustrates a block diagram of several major components of devices disclosed herein;

FIG. 2B illustrates a block diagram of several major components of devices disclosed herein;

FIG. 2C illustrates a block diagram of several major components of devices disclosed herein;

FIG. 2D illustrates a block diagram of the process of FIG. 1 wherein virtually all of the human intervention steps have been automated;

FIGS. 3A-E illustrate a fluid handling device from side and top views; FIGS. 3F-H show an exemplary interaction between layers illustrated in FIGS. 3A-E; FIGS. 3I-G illustrate top views of a fluid handling device; FIG. 3K illustrates use of a spindle to lift layers; FIG. 3L shows alignment of an outlet with an inlet; FIGS. 3M-N illustrates collection compartment;

FIG. 4 illustrates an alternative embodiment of a fluid handling device;

FIG. 5A illustrates a perspective view of a fluid handling device; FIG. 5B is a side-view of the device of FIG. 5A; FIG. 5C is a top view of a fluid handling device; FIG. 5D is a cut-out through a vertical plane of the device; FIG. 5E is a perspective view of the device separated into components; FIG. 5F illustrates the device being assembled; FIGS. 5G-S illustrate the device in perspective and cross-sectional views automatically or semi-automatically executing processes described in FIG. 1;

FIG. 6A is a perspective view of a device having an alternative form factor; FIGS. 6B-I illustrate the device of FIG. 6A in side-views, perspective views, top views and cross-sectional views being assembled and used;

FIG. 7A illustrates an alternative fluid handling device; FIGS. 7B-C illustrates independent horizontal and vertical motions of the layer which allows for alignment of compartments;

FIGS. 8A-B illustrate alternative shapes for compartment; FIGS. 8C-D illustrate a module configured to move droplets;

FIG. 9A shows an alternative reagent compartment; FIG. 9B illustrates an alternative pressure source configuration; FIGS. 9C-D illustrates an embodiment introducing reagents or samples into a reaction area column or vessel; FIGS. 9E-F illustrate an exemplary embodiment introducing reagents or samples into a reaction area column; FIGS. G-H illustrate reagent compartment configurations;

FIG. 10A illustrates an exemplary reagent compartment; FIG. 10B illustrates an embodiment suitable for introducing reagent samples into a solid phase vessel;

FIG. 11 illustrates an alternative column design with side walls;

FIG. 12A illustrates an apparatus adapted to manipulate a fluid processing device;

FIGS. 12B illustrates an automation of positional movement; FIG. 12C illustrates an apparatus with multiple slots; FIG. 12D shows a modular scalable system;

FIGS. 13-14 illustrate data showing the amount of DNA in each eluate quantitated by qualitative PCR for examples; and

FIG. 15A is a block diagram showing a representative example of a logic device through which dynamic a modular and scalable system shown in FIG. 12D can be achieved; and FIG. 15B is a block diagram showing the cooperation of exemplary components of a system suitable for use in a system where dynamic data analysis and modeling is achieved.

### DETAILED DESCRIPTION OF THE INVENTION

### I. Systems

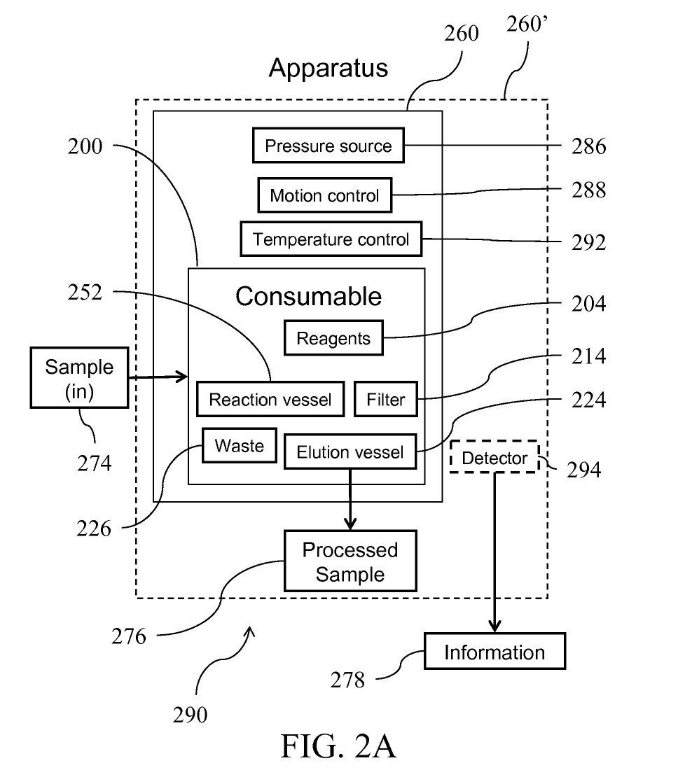

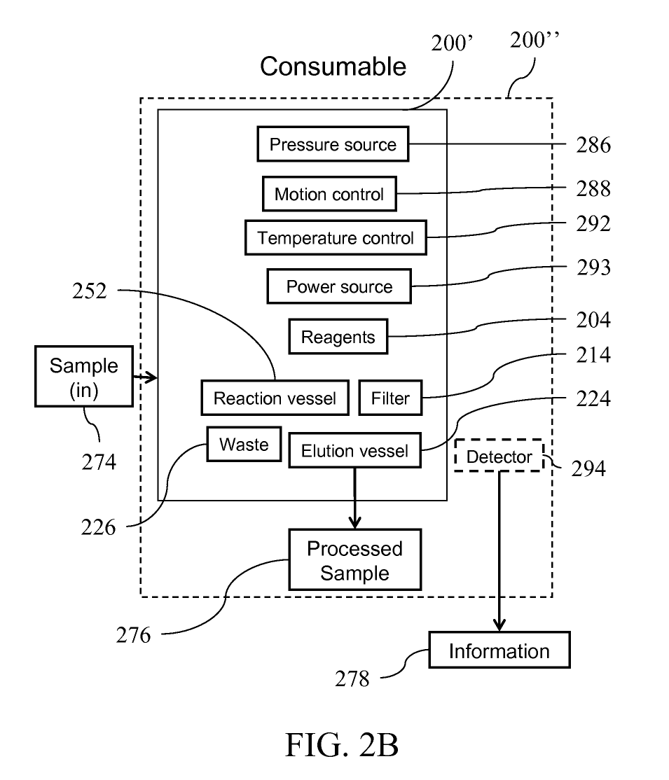

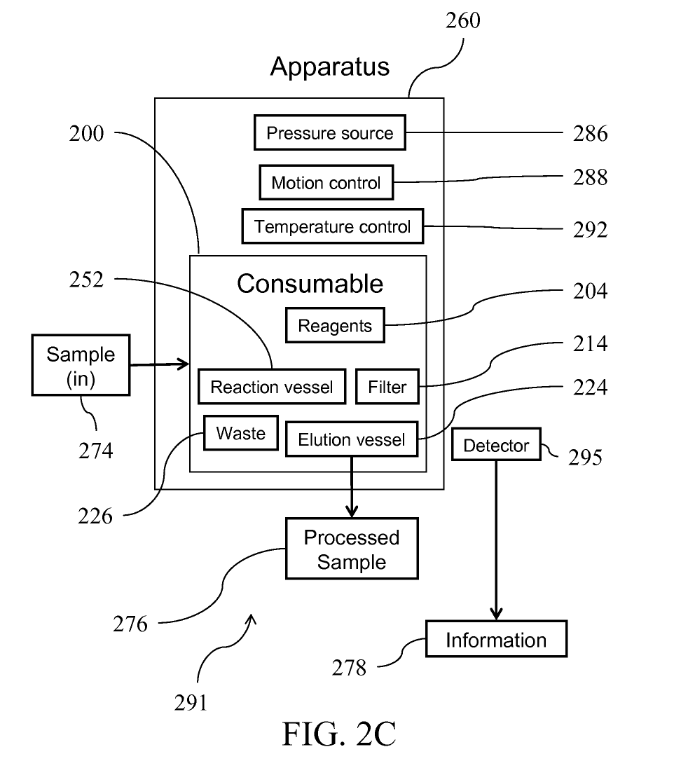

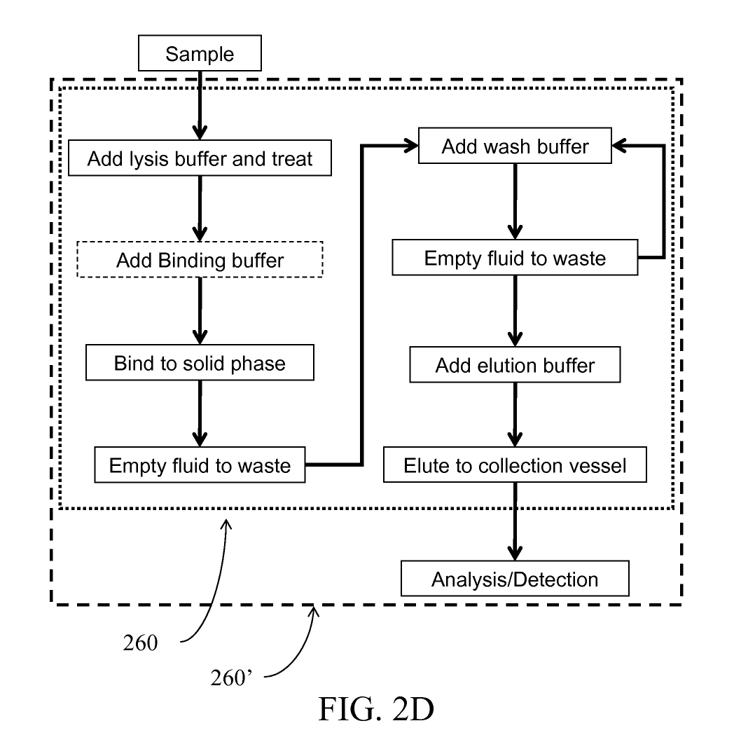

FIG. 2A illustrates a block diagram of several major components of a fluid handling system 290 suitable for use according to teachings of the disclosure. A fluid handling device 200 is provided that is configurable to contain on-board reagents 204. Typically the fluid handling device 200 is configured such that it forms a consumable unit and the reagents provided within the unit are consumed when the fluid handing device is used in a fluid handing system adapted and configured to perform various fluid processing steps on a sample using the fluid handling devices according to this disclosure.

The fluid handling device 200 can be configured such that a user can add one or more custom reagents as needed. Alternatively, the fluid handling device 200 can be configured to provide one or more reagents on-board while also providing one or more vessels that are accessible and/or customizable by a user to add one or more custom reagents. The fluid handling device 200 may contain one or more reaction vessels 252 to allow automated assembly of any reactions prior to the sample preparation steps. Fluid handling device 200 may also be configurable to contain one or more filters or filter columns 214 as a reaction area to facilitate processing of larger input sample volumes.

Fluid handling device 200 is further configurable to provide one or more of each of on-board waste collection vessel 226 and eluate collection vessel 224. As will be appreciated by those skilled in the art, providing on-board waste collection vessels and eluate collection vessels can further improve safety and reduce sample and processing contamination risks. The eluate collection vessel 224 may further be configured to contain reagents designed to carry out downstream reactions on the purified analytes.

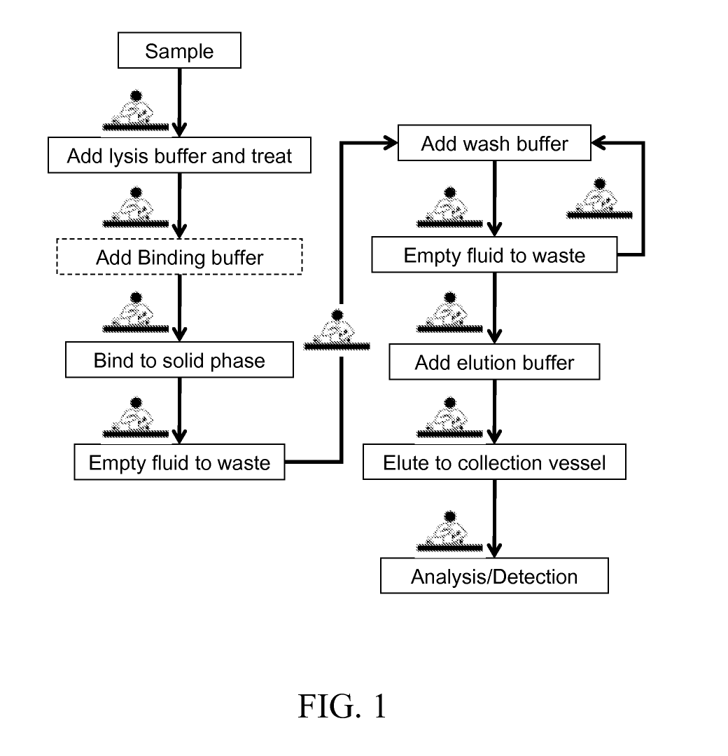

In use, the fluid handling device 200 is actuated by a fluid processing apparatus 260 which contains subcomponents suitable for use in carrying out one or more the fluid processing steps. The fluid handing device and the fluid processing apparatus together teach the fluid handing system. These subcomponents include, but are not limited to one or more of each of, pressure applicator 286, motion controller 288, and temperature controller 292. As shown, fluid processing apparatus 260 processes a fluid handling device 200 which receives an input sample 274 as provided by the user and, subsequent to processing, produces an output sample 276, such as purified nucleic acid (Sample to Processed Sample) wherein the sample had been processed without further human interaction with the sample during at least some of the processing steps shown on FIG. 1. Typically, the processed output sample 276 is contained by the eluate collection vessel 224. Suitable input samples 274 include, but are not limited to nucleic acids, blood, nasal washes, suspensions of particulates (such as dirt or feces), other cellular suspensions (such as saliva, cheek swabs, scabs, nail clippings, hair, buccal swabs), protein suspensions, mixtures of compounds and the like. Typical output samples 276 include, but are not limited to solutions of nucleic acids, proteins, carbohydrates, lipids, and/or chemical compounds.

As will be appreciated by those skilled in the art, if a reaction layer is configured to have more than one reaction area elements or columns, then a single sample could be split or separated into two (or more) different columns. For example, if the sample is added into the reaction vessel, then reacted with lysis buffer, the transfer of this material into the column could be split into two columns (or more). In this way you would have more than one output samples. Thus, as would be appreciated, there is no requirement that the columns are the same. One could be optimized to purify DNA and the other could purify proteins.

Still referring to FIG. 2A, fluid handling device 200 is configurable to carry out a wide variety of fluid handling processes substantially without human interaction such that the processing steps are performed automatically or at least semi-automatically. Accordingly, other alternative configurations of the fluid processing apparatus 260 forming part of the system designed to carry these varied fluid handling processes are possible. One alternative is shown as apparatus 260′. As illustrated, fluid processing apparatus 260′ contains subcomponents such as signal detector 294 to detect, for example, an output signal from any downstream reaction that may be generated. As shown, apparatus 260′ actuates a fluid handling device 200 which accepts or receives an input sample 274, produces a processed output sample 276 into elution collection vessel 224. Elution collection vessel 224 may contain prepackaged reagents which may execute a reaction on the processed output sample 276, producing a signal, which can be detected by signal detector 294, which outputs information 278 (Sample to Answer). Other suitable subcomponents can be used in the fluid processing apparatus without departing from the scope of the invention. By using the system described herein, the labor intensive and error prone processes described with respect to FIG. 1 can be converted into the robust and simple to use processes.

FIG. 2B shows an integrated Sample to Processed Sample fluid handling device 200′ which has integrated all necessary components needed to accept an input sample 274, execute various fluid processing steps to produce a processed output sample 276. These subcomponents include, but are not limited to one or more of each of, pressure applicator 286, motion controller 288, temperature controller 292, and optional on-board power source 293. The power source 293 may be for example one or more batteries. Other power sources can be employed without departing from the scope of the invention. Power sources include a battery, battery pack, rechargeable DC source, capacitor, or any other energy storage or generation (e.g., a fuel cell or photovoltaic cell) device known to those of skill in the art. The components of integrated fluid processing apparatus and consumable 200′ have been miniaturized and integrated where possible to provide a smaller overall form factor.

FIG. 2B show an integrated Sample to Answer fluid handling device 200″. Sample to Answer fluid handling device 200″ has integrated all necessary components needed to accept an input sample 274, execute various fluid processing steps to produce a processed output sample 276, and carry out necessary reactions and signal detection to output information 278. These subcomponents include, but are not limited to one or more of each of, pressure applicator 286, motion controller 288, temperature controller 292, optional on-board power source 293 (such as those discussed above, and detector 294. The components of integrated fluid processing apparatus and consumable fluid handling device 200″ have been miniaturized and integrated where possible to provide a smaller overall form factor.

FIG. 2C shows an integrated Sample-to-Answer fluid handling system 291 that uses the fluid handling device 200, and fluid processing apparatus 260, and detector 295. Fluid processing apparatus 260 manipulate the fluid handling device 200 and performs the necessary fluid handling process to convert the input sample 274 to a processed output sample 276 as described above. The processed output sample 276 is contained in elution vessel 224. Elution vessel 224 may contain prepackaged reagents which may execute a reaction on the processed output sample 276, which results in a signal detectable by signal detector 295. Examples of the detector 295 are commercially available instruments or consumable devices which may carry out temperature cycled or isothermal amplification. As would be appreciated by those skilled in the art, polymerase chain reaction (PCR) is just one example of a way to amplify nucleic acids using temperature cycling. Ligase chain reaction (LCR) is an example another way to amplify nucleic acids. LCR uses a ligase instead of a polymerase. There are other ways to amplify nucleic acids but at a constant temperature (isothermal) such as the Recombinase Polymerase Amplification (RPA). Additional detection can be provided through optical or electrochemical technologies, or nucleic acid lateral flow devices. Elution vessel 224 may be optimizable to mate specifically with the downstream commercial detector 295. These commercially available detectors have reaction vessels that have well defined form factors (test tube, microfluidic vessels, etc.) A Sample to Answer system could be constructed by “bolting” on apparatus 260 and forcing elution vessel 224 to adopt a compatible form factor for use in detector 295. The form factor of elution vessel 224 may be a test tube, a microfluidic device, or a nucleic acid lateral flow device as required by the specific detector 295. In addition, elution vessel 224 is configurable to contain reagents specifically required by detector 295. In some configurations, detector 294 is a customizable detector that is configured and incorporated into the apparatus 260′ or 200″ while detector 295 can be an off-the-shelf detector. As will be appreciated by those skilled in the art, elution vessel 224 could be customized to work well with detector 294.

Examples of commercially available detection apparatus that perform temperature cycled target amplification with optical signal detection are the MiniOpticon™ Real-Time PCR Detection System from Bio-Rad (Hercules, Calif.), the StepOne™ System from Applied Biosystems (Foster City, Calif.), and the Mx3005P® QPCR System from Agilent Technologies (Santa Clara, Calif.). These apparatuses are configured to accept test tube shaped vessels that receive processed samples (purified nucleic acids) and reagents that act to amplify nucleic acid targets and produce a signal that can be detected optically by the apparatus. A Sample to Answer system may be configurable by using the combination of fluid handling device 200, apparatus 260 and one of these commercially available apparatus that performs temperature cycled target amplification with optical signal detection. In this configuration, the elution vessel 224 would simply be the required test tube shaped vessel. Elution vessel 224 would be configurable to contain prepackaged reagents specifically required for temperature cycled target amplification and optical signal generation, for example, buffers, oligonuclotides, nucleotides, enzymes and fluorescent dyes. These reagents could be in a dry state (lyophilized) that become reconstituted upon the introduction of the processed sample by the combination of fluid handling device 200 and apparatus 260. An example of these reagents in a lyophilized state is the illustra PuRe Taq Ready-To-Go™ Beads by GE Healthcare (Waukesha, Wis.).

Another example of an apparatus that perform temperature cycled target amplification with optical signal detection is the 7900HT Fast Real-Time PCR System from Applied Biosystems (Foster City, Calif.). In addition to using test tube shaped reaction vessels, this instrument is also capable of using a Custom TaqMan® Array, a 384 well microfluidic card that allows samples to be run against TaqMan® Gene Expression Assay targets that are preloaded into each of the wells on the card. A Sample-to-Answer system may be configurable by using the combination of fluid handling device 200, apparatus 260 and an apparatus similar to the 7900HT Fast Real-Time PCR System. In this configuration, the elution vessel 224 would simply be a microfluidic card similar to the Custom TaqMan® Array.

Other commercially available detection systems utilize different detection technologies. For example, the TruDiagnosis® Systems from Akonni Biosystems (Frederick, Md.) uses a TruCycler™ Thermal Cycler to amplify the nucleic acid in a TruArray® MicroArray, followed by a TruDx™ Reader to detect the signal. The vessel required for the TruDiagnosis® Systems is the TruArray® MicroArray, which is a microfluidic vessel containing a micro-array of gel-drop biosensors. A Sample-to-Answer system may be configurable by using the combination of fluid handling device 200, apparatus 260 and an apparatus similar to the 7900HT Fast Real-Time PCR System. In this configuration, the elution vessel 224 would simply be a microfluidic card similar to the TruArray® MicroArray.

Another detection technology is the Lateral Flow device exemplified by pregnancy test devices. These devices can be adapted to detect proteins, nucleic acids or compounds. A Sample-to-Answer system may be configurable by using the combination of fluid handling device 200, apparatus 260 and a lateral flow device where the elution vessel 224 is the lateral flow device.

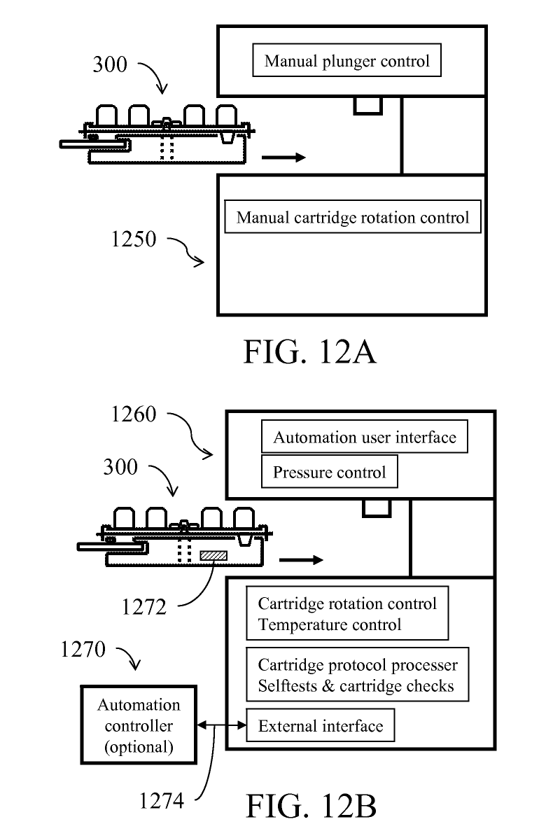

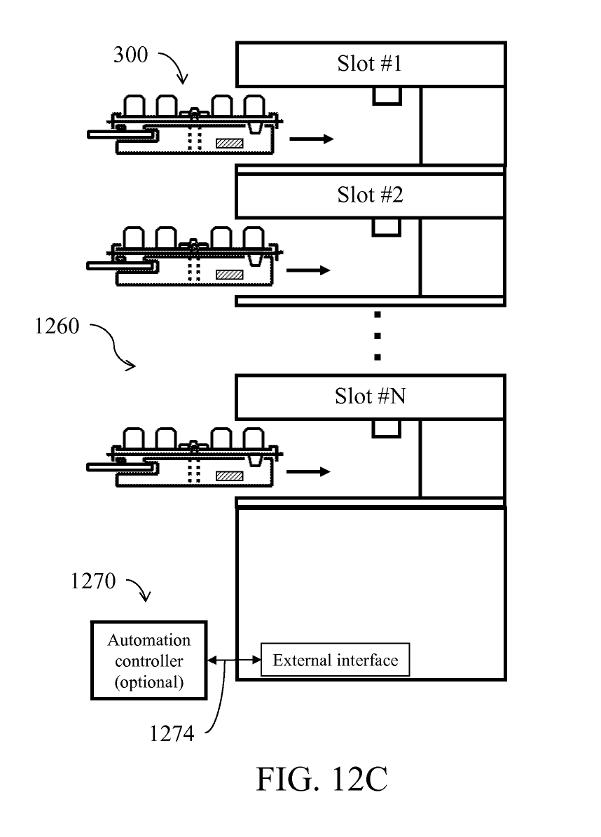

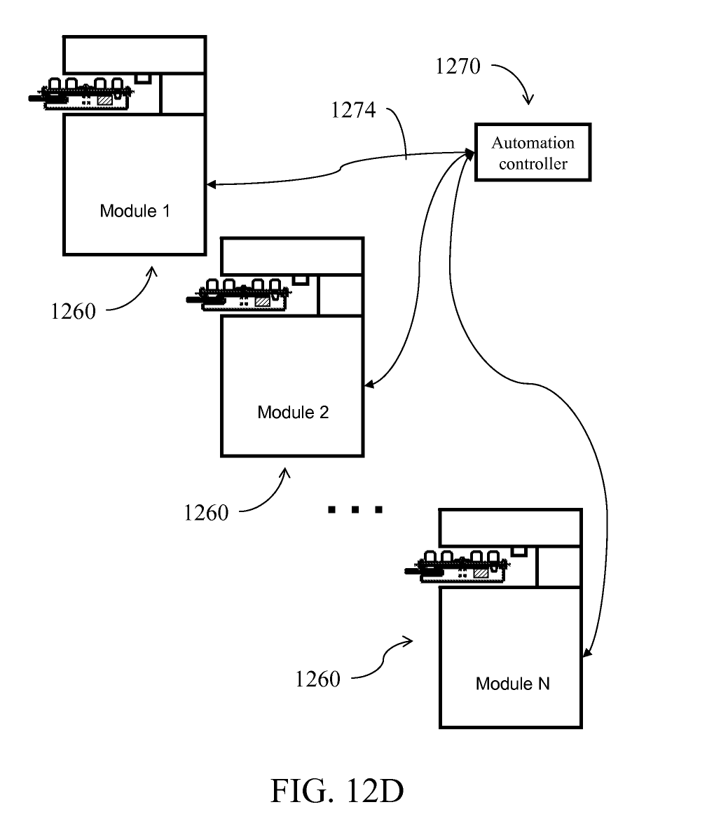

Persons skilled in the art would appreciate that by modifying the form factor of the elution vessel 224, the fluid handling device 200 and the fluid handling apparatus 260 may be coupled with other detection instrumentation. Persons skilled in the art would also appreciate that such an integrated apparatus may be used alone or as part of a larger system as shown in FIG. 12B-D. Information available from the integrated apparatus includes results from the detection process and status of both the sample preparation and detection process.

By using the system described herein, the labor intensive and error prone processes described with respect to FIG. 1 can be converted into the robust and simple to use process in FIG. 2D where many of the steps requiring human interaction have been automated. The dotted box 260 shown in FIG. 2D encompasses a process executable by the Sample to Processed Sample apparatus. The dashed box 260′ shown in FIG. 2D encompasses a process executable by the Sample to Answer apparatus. Aspects of the disclosure are adapted and configured to carry out sample processing with these advantageous features.

### II. Fluid Handling Devices

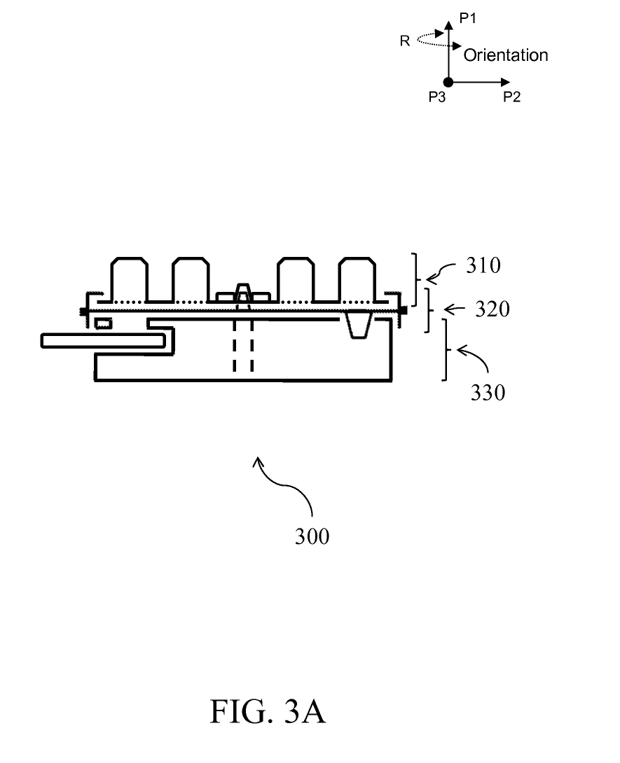

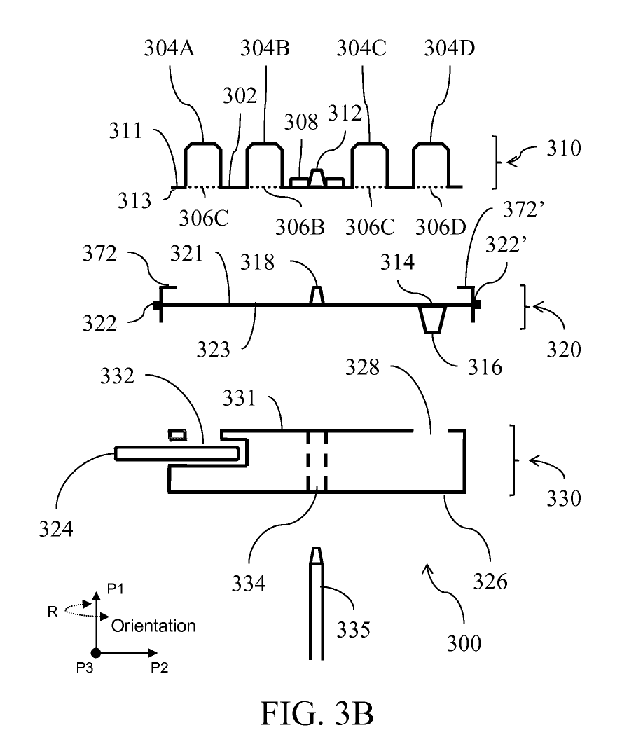

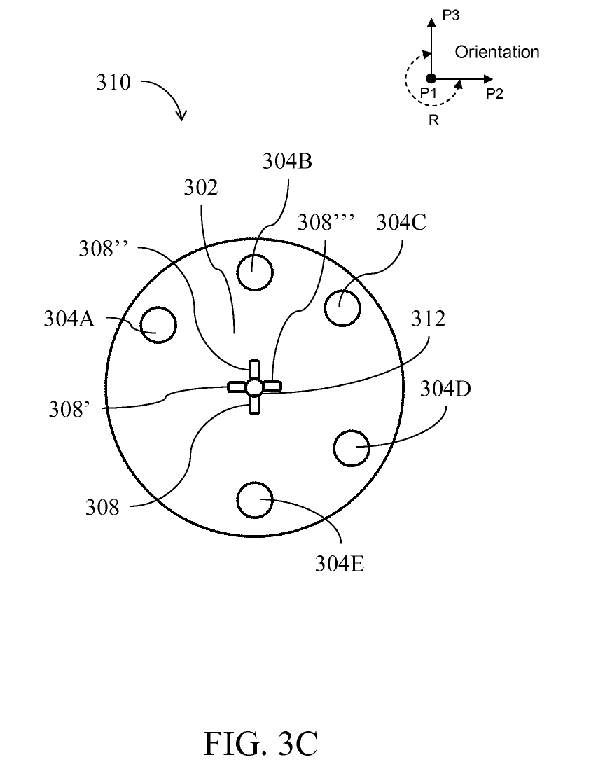

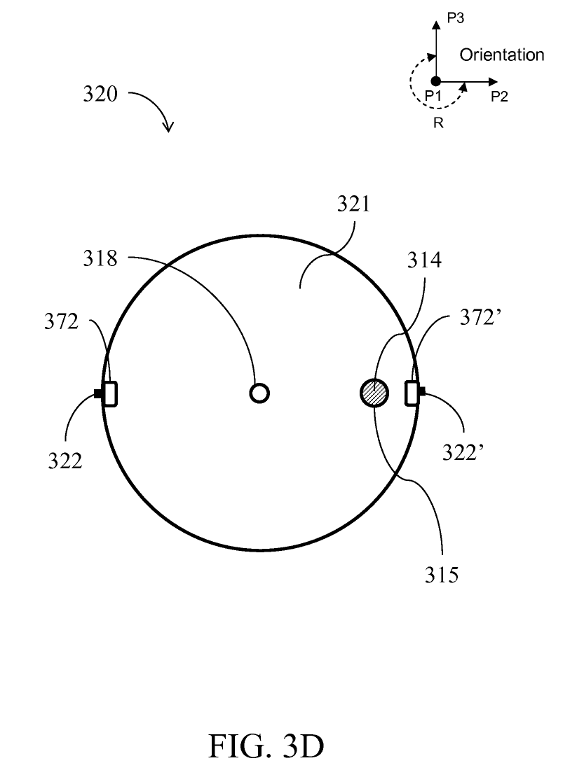

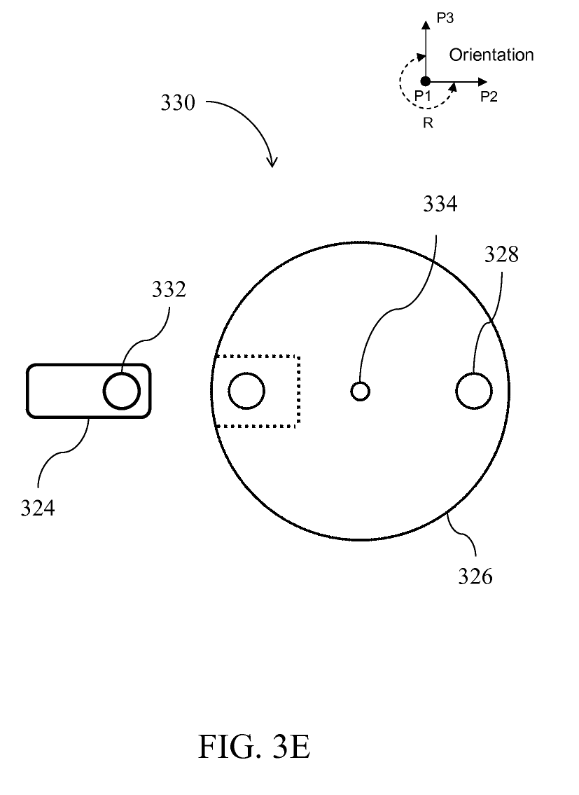

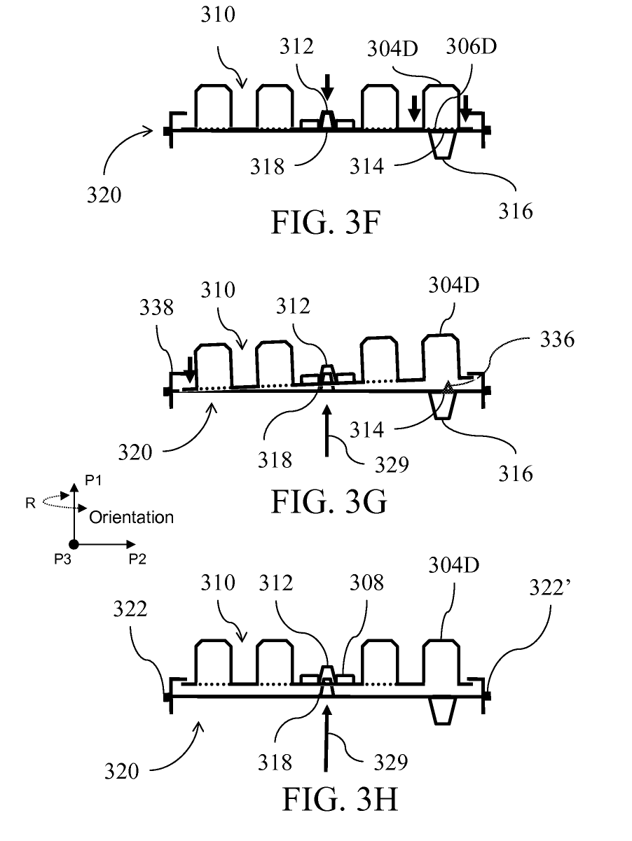

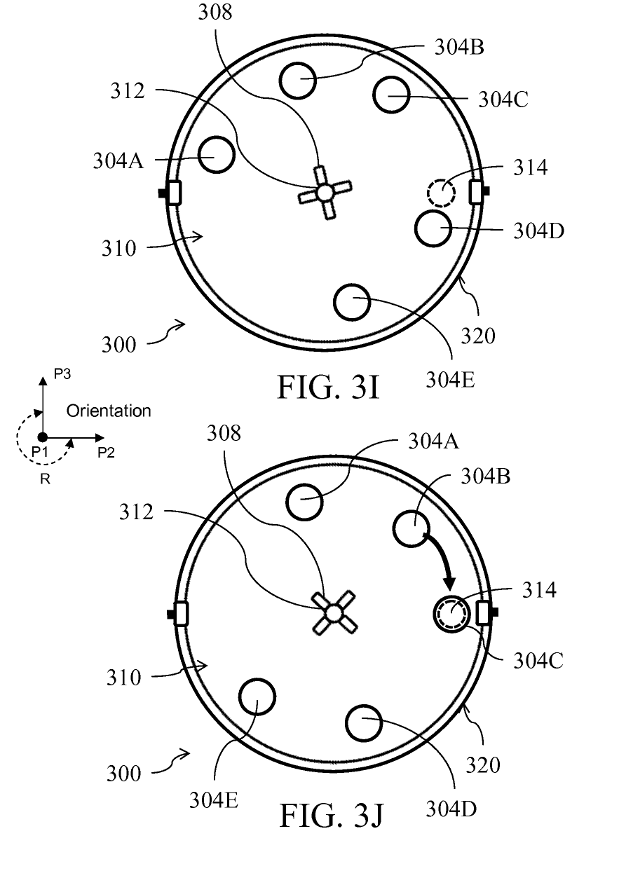

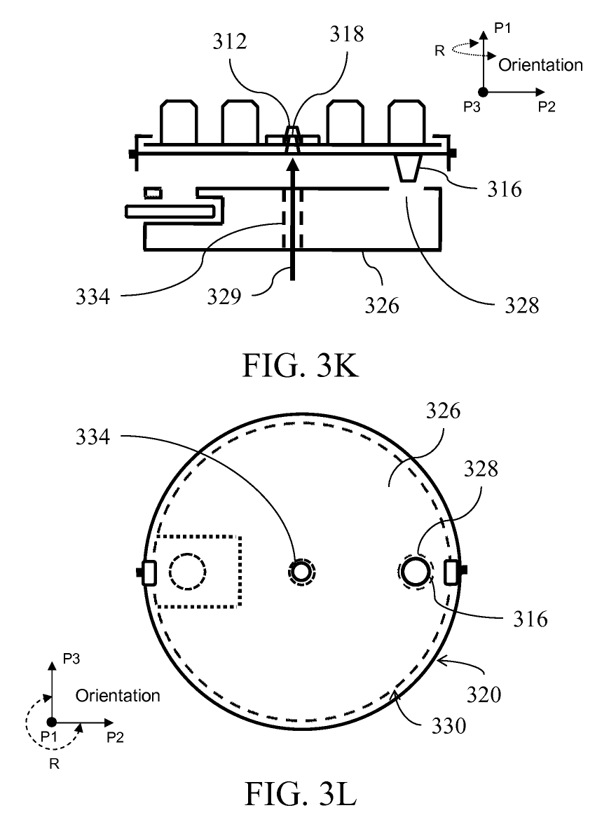

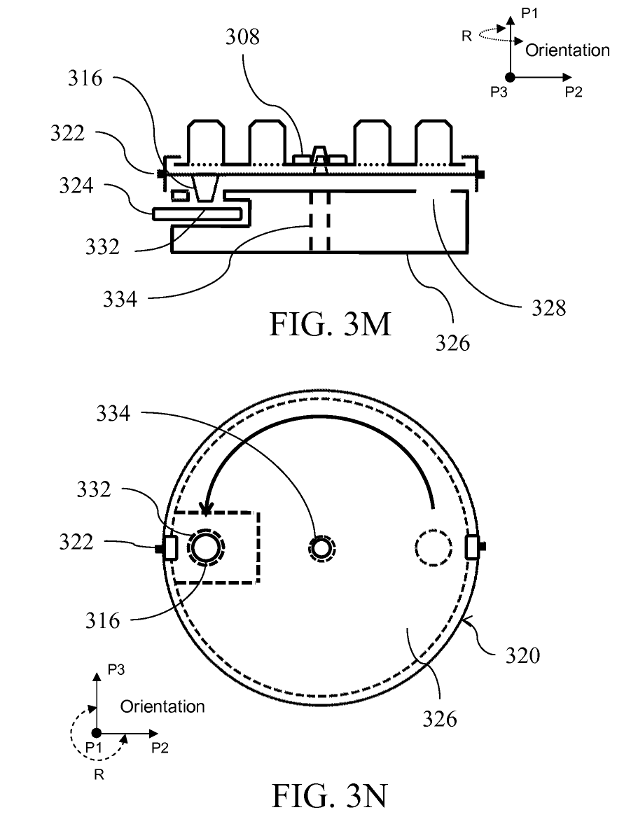

An embodiment of a fluid handling device 300 suitable for use in accordance with the disclosure herein is shown in FIG. 3A. The fluid handling device is composed of, for example, three layers: a reagent layer 310, a reaction layer 320 and a fluid collection layer 330. As will be appreciated by those skilled in the art, fewer layers can be used without departing from the scope of the invention.

The fluid handling device 300 is adapted and configured to fit into and removeably mate with a fluid processing apparatus (see e.g., FIG. 2) which has mechanical mating which interacts with positioning features on the fluid handling device. Suitable mechanical mating features would be readily appreciated by those of skill in the art and include, but are not limited to the use of notches, apertures, grooves, slits, slots, detents, and features configured to achieve male-female or female-male mating between the fluid handling device and the fluid processing device.

FIGS. 3B-E illustrate side and top views of reagent layer 310, reaction layer 320 and fluid collection layer 330 of the fluid handling device 300. FIG. 3B illustrates a cross-section of the fluid handling device 300 in an exploded view such that each of the layers is separated. Reagent layer 310 is substantially planar and has a first surface (shown as an upper surface) 311 and a second opposing surface (shown as a lower surface) 313 and can be configured such that it contains one or more reagent compartments 304A, 304B, 304C, 304D extending from the first surface wherein each reagent compartment is adapted and configured to form a vessel suitable for housing one or more fluids, arranged on a reagent layer substrate 302. These reagent compartments 304 may be of variable sizes and shapes to facilitate accommodating or holding different amounts of fluid. The ability to provide variable sized reagent compartments may be particularly useful where different fluid quantities are required for a particular processing and the processing fluids should be maintained without contact to air. These reagent compartments are at least partially compressible and contain individually or in combination prepackaged liquid, solid reagents, components added by the user, and gases such as atmospheric gases, oxygen, or nitrogen or other suitable gases and gaseous combinations. Where a plurality of primary reagent storage compartments 304A, 304B, 304C, 304D is used, the compartments are connectable by, for example, a channel, which may be enclosed, between two or more compartments (not shown) placing them in fluid communication. Persons of skill in the art would readily appreciate how to construct a channel between compartments. Alternate compression and expansion of these primary reagent storage compartments 304A, 304B, 304C, 304D will cause fluid flow and mixing of the contents between compartments. These primary reagent storage compartments 304A, 304B, 304C, 304D are further sealable with one or more pressure frangible seals 306A, 306B, 306C, 306D. An example of a suitable seal is an aluminized multilayer lidding commonly found in pharmaceutical drug or food packaging. This material will tear and release its content when pressure is increased by compression of the one or more reagent compartments 304A, 304B, 304C, 304D. Pre-scoring of the frangible lid can create a weakened area that will fail preferentially over un-weakened areas.

Other suitable seals, such as duck billed valves, or snip-off or tear-off seals to can be used without departing from the scope of the invention. In the case of duck billed valves the entire second surface 313 of reagent layer 310 is coverable with aluminum lidding to improve reagent retention during storage. This lidding is removable by the user or by the fluid processing apparatus prior to use. In the case of tear-off seals, mechanical features on the fluid processing apparatus may open these seals, for example by cutting, twisting, or tearing the seal, prior to compression of the compartment.

A first support 312 forming a male conical support on the first surface 311 of the reagent layer 310 and a female receiving aperture on its second surface 313 can extend from a reagent layer first surface 311 of the planar substrate 302 of the reagent layer 310. The first support 312 is further configurable to enable the reagent layer 310 to freely rotate R about an axis P1 extending out of the plane P2-P3 as further shown in FIG. 3C. As further illustrated in FIG. 3C, reagent layer 310 substrate 302 is configured to be circular in cross-section across the plane P2-P3. Shoulder features 308, 308′, 308″, 308′″ which extend radially in plane P2-P3 from the upright first support 312 and protrude from the planar substrate 302 on the first surface 311 are grippable either manually or by a fluid processing apparatus to apply rotational force to the reagent layer 310.

The reaction layer 320 having a first (upper as depicted in FIG. 3B) side 321 and a second (lower as depicted) side 323 can contain a reaction area 314 compatible with a reaction area outlet 316 positioned to extend from the second surface 323. As shown in FIG. 3D, which illustrates the reaction layer 320 from the first surface 321, a second support 318 extends from the center of the reaction layer 320 and an aperture 315 in reaction layer 320 which provides fluidic access to the reaction area 314 (hatched). Examples of this reaction area 314 can be filters which can filter fluids or can bind subcomponents of a fluid. This reaction area can also be a solid phase such as beads which rest on a frit to prevent their escape. Second Support 318 extends from the first surface 321 of the on reaction layer 320 and is configured to provide a male surface adapted to mate within the female receiving aperture of the first support 312 on the second surface 313 of the reagent layer 310 and to nest inside the first support 312 on reagent layer 310. The mated second support 318 within the first support 312 further enables free rotation of reagent layer 310 and reaction layer 320 either simultaneously or at different rates of rotation about axis R. Second Support 318 is further configured to provide a female receiving aperture on its second surface 323. Fluid processing apparatus spindle 335 which can pass through spindle hole 334 of fluid collection layer 330 is configured to provide a male conical surface adapted to mate within the female receiving aperture of first support 312 and second support 318. Vertical motion along axis P1 and/or rotational motion R about axis P1 of spindle 335 can provide lifting and rotational force to reagent layer 310 and/or reaction layer 320. Aligning two or more positioning features such as lateral pins 322, 322′ that extend radially from the reaction layer 320 (e.g., from at least a first side and a substantially opposing second side, or three lateral pins separated by approximately 120° on a circular substrate) allows them to be gripped manually or by a fluid processing apparatus to provide vertical motion and/or to provide rotational force for the reaction layer 320 or to hold in a fixed position. Reaction layer 320 can further be adapted to contain latch 372, 372′, adapted and configured to engage at least a portion of an outer edge of the reagent layer 310 and can act to engage reagent layer 310 and reaction layer 320 such that while they retain relative independent motions, the layers can remain in an engaged configuration during shipment, use and/or disposal. Similar latch features (not shown) can be provided on fluid collection layer 330 to facilitate maintaining the reaction layer 320 and fluid collection layer 330 in an engaged configuration during shipment, use and/or disposal.

The fluid collection layer 330 is made up of one or more fluid collection compartments 324, 326. The one or more fluid collection compartments 324, 326 can each have corresponding inlets 332, 328 which face toward the second surface 323 of reaction layer 320. Free rotation, R, of the reaction layer 320 about an axis P1 extending out of the plane P2-P3 as further shown in FIG. 3C, can place reaction area outlet 316 (which extends downward from the second side 323 of reaction layer 320) in fluid communication with fluid collection compartments 324 or 326 via their respective inlets 332, 328 on a first surface 331 of the fluid collection layer. The shape and dimension of fluid collection compartments 324, 326 are arbitrarily shown in the figures for purposes of illustration. As will be appreciated by those skilled in the art, the fluid collection compartments 324, 326 can take on a variety of configurations without departing from the scope of the disclosure. Examples of suitable compartment configuration include, but are not limited to, rigid compartments as shown, flexible bags that expand with increasing fluid volumes, microfluidic devices, or simple tubes that are common to the typical laboratory environment. Fluid collection compartments designed to receive purified analytes may also be further optimized for use as a detection device. For example, lyophilized enzymes, buffers and other reagents may be prepackaged in these compartments, which are then reconstituted upon addition of an analyte containing fluids. Non-limiting examples of reconstituted detection reactions include the 5′ fluorogenic exonuclease assay (U.S. Pat. No. 5,210,015, the recombinase polymerase amplification reaction (U.S. Pat. No. 7,485,428), and the ligase chain reaction (U.S. Pat. No. 6,054,564). These reactions are but a few examples of reactions that can specifically convert a small number of target analytes into a detectable signal. The fluid processing apparatus may contain subcomponents to thermally regulate [isothermal or temperature cycled for example]the fluid collection compartments to ensure optimal conditions for the requisite detection reaction. The fluid processing apparatus may also be configured to contain detection subcomponents or be in communication with detection subcomponents in order to detect the signals generated from these reactions. Non-limiting examples of these signals include fluorescence, colorimetry, and electrochemical detection. Other suitable means to convert target analytes to a detectable signal and to detect that signal can be used without departing from the scope of the invention.

FIGS. 3F-H show exemplary interactions between the reagent layer 310, and the reaction layer 320. As described earlier, reagent layer 310 contains central support 312 and reaction layer 320 contains central support 318. Reaction layer central support 318 is configured to extend from the first surface of the reaction layer 320 and for the first surface of the central support to nest inside the second surface of support 312 which is configured to form a female mating surface for the male reaction area central support as shown in FIG. 3F. A spindle 335 (shown in FIG. 3B), associated, for example, with the fluid processing apparatus, can be moved “up and down” along an axis providing motion 329. The spindle 335 (shown in FIG. 3B) may further include and use, for example, protrusions and/or gear teeth to selectively interlock and rotate the layers of the fluid handling device to accomplish desired fluid processing steps. A wider spindle portion may contact and lift reaction layer 320 at reaction layer central support 318 and a narrower spindle portion may extend through reaction layer central support 318 to contact and lift the reagent layer 310 at reagent layer central support 312. In this manner, reagent layer 310 and reaction layer 320 may be rotated together or independently. When the fluid handling device 300 is inserted into the fluid processing apparatus the fluid handling device 300 comes into communication with fluid processing apparatus spindle 335 (shown in FIG. 3B). The fluid processing apparatus can provide rotation R and motion along an axis, e.g., P1, during the fluid handling process to reagent layer 310, reaction layer 320 and fluid 330. The spindle 335 may act just to maintain concentricity of a plurality of device layers while a separate lift and rotate mechanism (not shown) grab the outside of one or more of the device layers for example by grabbing positioning features 322 and 322′ to provide vertical and rotational motion. Alternatively the spindle 335 may provide motion in two axes (e.g., vertical and rotational). Also alternatively the motions may be shared between the spindle 335 and a second motion control mechanism (not shown) in the fluid processing apparatus.

The spindle 335 furthermore can be integrated with the fluid handling device 300, integrated with the fluid processing apparatus, or split between the fluid handling device and the fluid processing apparatus. One advantage of having a spindle integrated with the fluid handling device 300 is the unification of the fluid handling device 300 and maintenance of concentricity of reagent layer 310, reaction layer 320, and fluid collection layer 330. Persons skilled in the art would appreciate there are different ways of designing the spindles 335 for fluid processing apparatus and fluid handling device 300 while providing independent motion of reagent layer 310, reaction layer 320 and fluid collection layer 330 without departing from the scope of the invention.

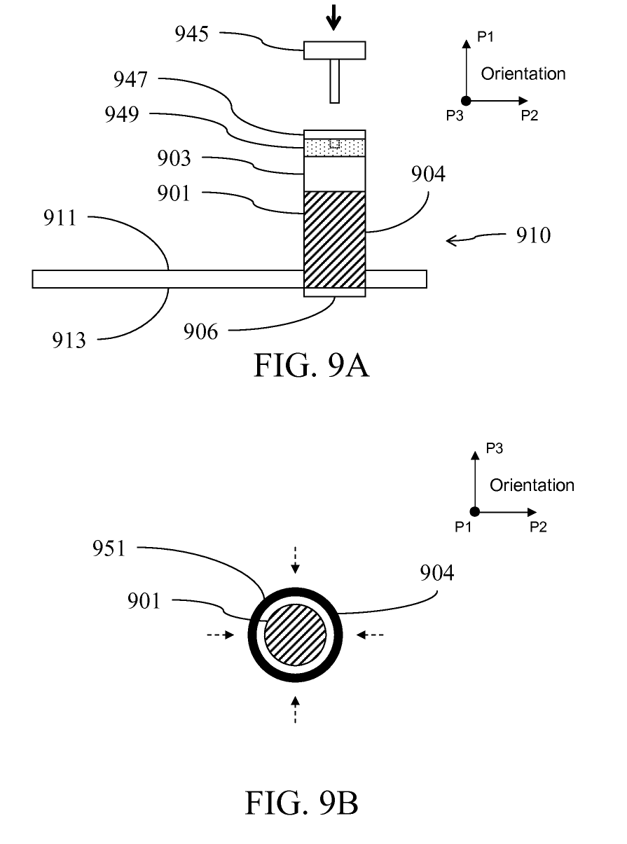

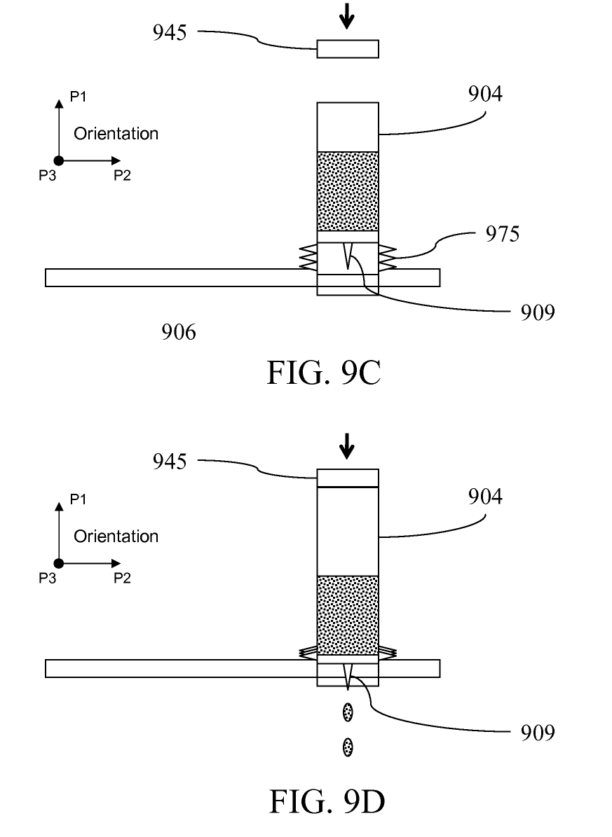

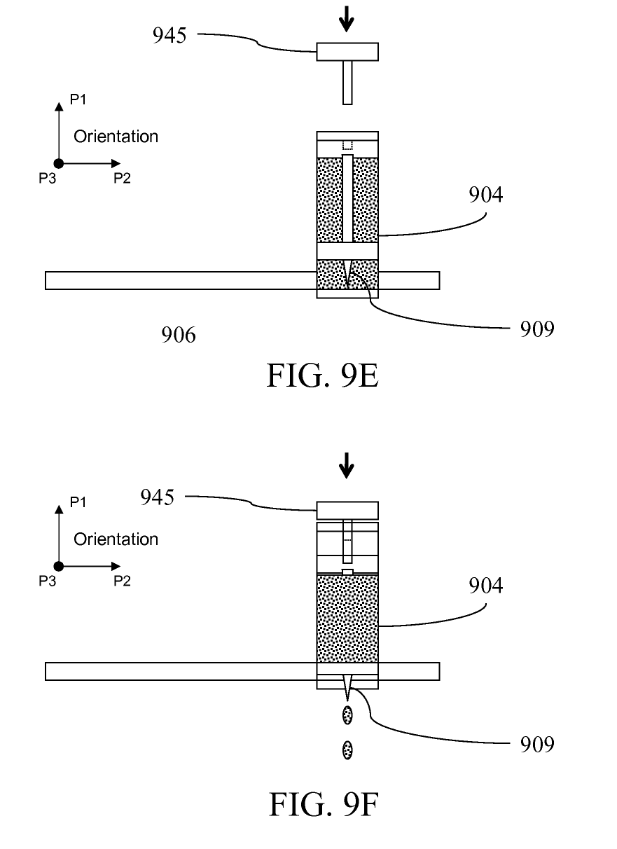

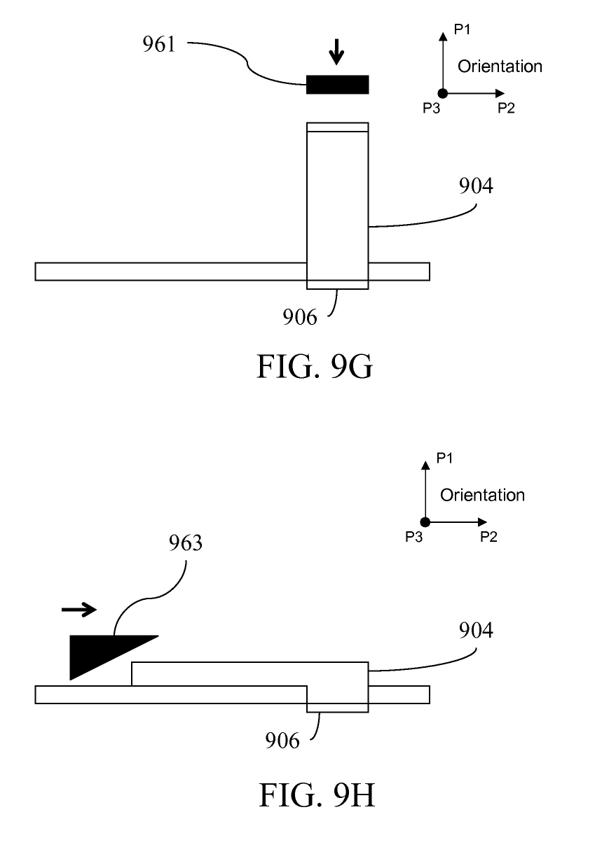

Independent rotations, R, of reagent layer 310 and reaction layer 320 about axis P1 extending out of the plane P2-P3 as further shown in any of FIGS. 3C, 3I, 3J alignment of any target reagent compartment 304A, 304B, 304C, 304D on reagent layer 310 can be achieved with respect to the reaction area 314 on reaction layer 320. Once a reagent compartment 304 has been aligned with the reaction area 314, application of pressure at the indicated points (arrows in FIG. 3F) will ensure pressure tight sealing between the pressure frangible seal 306 of a reagent compartment 304 and the reaction area 314. Reaction layer 320 may contain guide features and O-rings to help guide the alignment of a primary reagent storage compartment 304 of the reagent layer 310 with the reaction area 314 to ensure a pressure tight seal. Reaction layer 320 may also contain sharpened points 336 (shown in FIG. 3G only for clarity) positionable under seal 306 to ensure puncturing of seal 306 to release the fluid in reagent compartment 304. Other mechanisms suitable for use to cause or aid in the failure of seal 306 are shown in FIGS. 9C-F. Other suitable mechanism can be provided which are adapted and configured to cause or aid in the failure of seal 306 can be used as will be appreciated by those skilled in the art without departing from the scope of the invention.

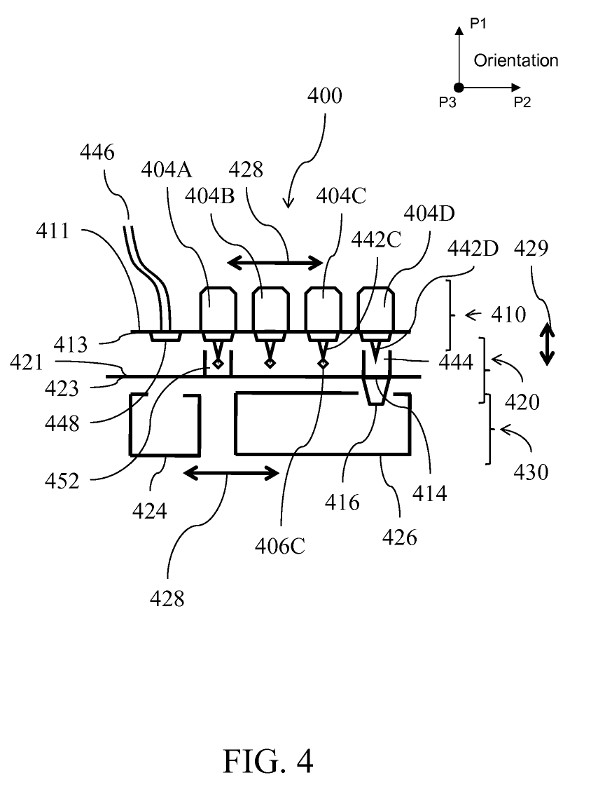

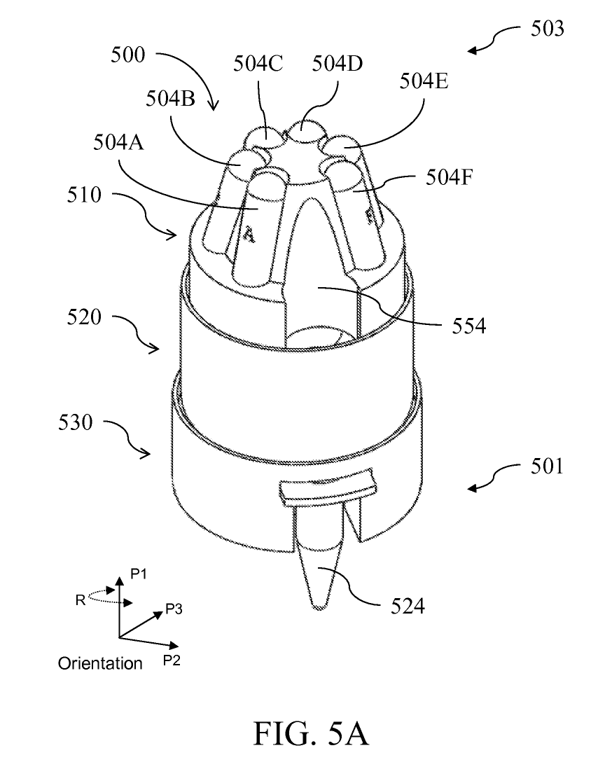

Once a pressure tight seal has been formed between pressure frangible seal 306 and the reaction area 314, compression of reagent compartment 304 will generate sufficient pressure to break seal 306, deliver its content through reaction area 314, where upon the effluent will flow through reaction area outlet 316 into a fluid collection layer 330 (shown in FIGS. 3A-B). Alternatively, the effluent can flow into one or more collection compartments that are a part of the fluid processing apparatus and not part of the fluid handling device itself. If a reagent compartment 304 contains air or other gases, cycles of compression and decompression of the reagent compartment will draw air back and forth across the reaction area 314, thus drying the reaction area 314. Alternatively, one or more of the reagent compartments 304 are replaceable by a fitting (not shown) which can form a tight seal with reaction area 314. Persons of skill in the art would appreciate that suitable fittings include, for example, those discussed and shown herein, such as fitting 448 discussed in FIG. 4. This fitting is attachable to a liquid reagent or gas source from the fluid processing apparatus to deliver a greater volume of reagent or gas to the reaction area 314 of the fluid processing apparatus than is practical with an enclosed compartment 304 provided with the fluid processing apparatus.

After evacuation of reagent compartment 304, it may be desirable to break the seal between 306 and reaction area 314 to allow access to a new, previously unused, or partially used reagent compartment. FIG. 3G shows how upward pressure from the spindle, associated with the fluid processing apparatus (not shown) acting on the second surface 313 of support 312 of reagent layer 310, along with a simultaneous downward pressure on the first surface 311 from the fluid processing apparatus on reagent layer 310 at a point along the substantially planar layer substantially opposing that of the reaction area 314 can impart a seal breaking force to separate the seal between frangible seal 306 and the reaction area 314. FIG. 3G and FIG. 3H show reagent compartment 304D after the associated seal 306D had been broken and the content of reagent compartment 304D passed through reaction area 314.

Reagent layer 310 is separable from reaction layer 320 by application of a pressure from, for example, the spindle 335, associated with the fluid processing apparatus (not shown), into central support 312 (FIG. 3H) of the reaction layer 320. The fluid handling device may use one or more positioning features 322, 322′ to maintain reaction layer 320 stationary, or relatively stationary, relative to a lifting action of the spindle on reagent layer 310. Once reagent layer 310, and reaction layer 320 are separated, the fluid processing apparatus (not shown) can use shoulders 308 to impart vertical motion and/or a rotational force about an axis P1 on reagent layer 310. FIGS. 3I and 3J show top views of fluid handling device 300 and illustrates how rotation R about axis P1 extending out of the plane P2-P3 of reagent layer 310 allows alignment reagent compartment 304C with reaction area 314 on reaction layer 320. In a similar manner, axial rotation of reagent layer 310 allows alignment of any reagent compartments, 304A, 304B, 304C, 304D with reaction area 314 on reaction layer 320.

Reagent layers 310 and reaction layer 320 are independently rotatable of fluid collection layer 330. FIG. 3K shows use of the spindle 335, associated with the fluid processing apparatus (shown in FIG. 3B), to provide motion 329 to lift reagent layer 310 and reaction layer 320 such that reaction area outlet 316 clears the fluid collection inlet 328 of fluid collection compartment 326 on the fluid collection layer 330. FIG. 3L shows alignment of reaction area outlet 316 with fluid collection inlet 328. Rotation of reagent layer 310 and reaction layer 320 relative to fluid collection layer 330 allows alignment of reaction area outlet 316 to a fluid collection inlet 332 of a different fluid collection compartment 324, as shown in FIGS. 3M-N.

It is possible to include other features into the preferred invention to improve the functionality of the device. For example, it may be desirable to include absorbent materials (not shown) in fluid collection compartment 326 to absorb any liquid waste received from reaction area outlet 316. In addition, through independent motions of fluid collection layer 330 and reaction layer 320, reaction area outlet 316 may be raised up and down to touch this absorbent material in order to wick off any liquid that may adhere to reaction area outlet 316. Filter materials (not shown) may be placed around the junctions of reagent layer 310, reaction layer 320 and fluid collection layer 330 to minimize the escape of aerosols, particulates or gases from the fluid handling device. After use, escape of reagents or waste can be minimized and ease of disposal can be maximized by heat or adhesive sealing of layer reagent layer 310, reaction layer 320 and fluid collection layer 330 to each other.

One or more each of the reagent layer 310, reaction layer 320 and fluid collection layer 330 can further be configured to include one or more mechanisms (for example, such as unique device identification 1272 discussed in FIG. 12B) for providing information relating to that layer and/or the fluid handling device generally including, for example, a unique device identification component. Suitable information or unique identification includes, for example, serial numbers, information relating to manufacturer, serial number(s), lot number(s), date codes (e.g. manufacture date, expiration date, etc.), reagent types, reagent volumes, reaction area type, process ID (i.e., the type of process that will be performed and/or the method or sequence of performing steps for a particular set of reagents), process parameters needed to run the process (temperature, pressures, timing), calibration parameters. Additionally, the information mechanism can be configured to communicate with other layers (e.g., to ensure suitability of combining layers) and/or with the fluid processing apparatus.

In one aspect, bar codes can be used to store limited information about an layer. For example, such bar codes are oftentimes positioned on the layer itself, and configured to include static information (see for example U.S. Pat. No. 6,180,351). However, as will be appreciated by those skilled in the art, while effective at storing certain data, bar codes have some limitations. For example, bar codes are not capable of collecting dynamic information. In fact, if information is to be added after the bar code has been configured, such as information related to the processing, or if information must be changed or erased, the bar code must be replaced by another bar code which has had the new information transposed thereupon. In other words, information contained on bar codes is fixed as of the time the bar codes are made and placed on the layer or device, which is typically at the point of fabrication. Second, the bar codes are limited to the amount of information they can store because of size constraints. For example, unique identifiers which match a layer to its specific layout information, often referred to as “Globally Unique Identifiers” or “GUIDs” or “Universally Unique Identifiers” or “UUIDs”(see for example U.S. Pat. Nos. 5,812,793 and 5,404,523) typically require 128 bit data string. However, a string of such a length when written as a bar code would usually take up about 3 to 4 cm, which is more room than is often available on a substrate adjacent a typical array (which may be less than about 1 cm in any dimension). Thus, oftentimes a second, shorter code is used, where such a shorter code is used to identify the actual unique identifier. However, this technique adds complexity to the array process. Third, a bar code requires the use of a bar code scanner for reading the information contained on the bar code itself. Such ancillary equipment adds to the cost and complexity of data retrieval.

Additionally, a data storage element can be provided in addition to a bar code which is configurable to receive and store large amounts of data. Suitable data storage elements include, for example, magnetic, silicon chip, optical or solid state storage devices (including magnetic or optical disks or tape or RAM, or any other suitable device). In other words, the data storage element is capable of storing a greater amount of data than would be feasible to store on an bar code and will typically have a storage capacity of from one byte to hundreds of bytes of data to multiple tens or even hundreds of megabytes of data or more. As such, typically about 100 bytes to about 500 megabytes of data or more may be stored, usually from about 250 bytes to about 15 megabytes of data may be stored and more usually from about 0.125 megabytes to about 4 megabytes of data may be stored by the data storage element. Data may be stored in the data storage element manually, for example in the case of static data, or automatically, for example in the case of dynamic data. The stored data may be organized into separate or discrete areas. For example, data may be stored in areas that are generally or broadly accessible and/or stored in areas that are secure or protected, i.e., areas that have limited accessibility, e.g., the areas are protected and accessible only if a password is provided, or the like. Furthermore, the data may be stored in a variety of formats, including, but not limited to, raw, processed, encrypted and decrypted formats. In certain embodiments of the subject invention, certain data may be stored in a generally accessible area and certain other data may be stored in a limited access area, where some or all of the data stored in either or both of the generally accessible areas and/or limited access areas may be raw and/or processed and/or encrypted and/or decrypted.

Devices configured to collect, receive and store at least one of static and dynamic data, where such data can be easily and securely communicated, i.e., transferred to or received from, at least one external or remote apparatus or site such as a fluid handling apparatus, a device detector, a personal computer (“PC”), and the like. The data storage element may be positioned in or on any or all of the layers of the device or associated with the device generally. See, for example, U.S. Pat. Nos. 6,238,910; 5,958,760; and 6,114,122 and U.S. Patent Publication No. US 2005/0063227 A1.

FIG. 4 shows an alternative exemplary fluid handling device 400 where reagent compartments 404A, 404B, 404C, 404D, residing on, for example, a first surface 411 of a reagent layer 410, have tips 442A, 442B, 442C, 442D with tear off frangible seals, 406A, 406B, 406C, 406D at the tips. For clarity, only tips 442C, 442D and frangible seal, 406C are labeled on the FIG. 4. The tips 442A, 442B, 442C, 442D extend (downward in this configuration) from, for example, a second surface 413 of reagent layer 410 and are in fluid communication with their respective reagent compartments, 404A, 404B, 404C, 404D. Reaction layer 420 has a first surface 421 and an opposing second surface 423 arranged below reagent layer 410 in communication with an opposing second surface 413 of reagent layer 410. Reaction layer 420 contains reaction area compartment 444, reaction area 414, and reaction area output 416. Reaction area compartment 444, arranged on first surface 421, is open at the top and is in communication with an opposing second surface 413 of reagent layer 410. Reaction area output 416, arranged on second surface 423, is open at the bottom and is in communication with fluid collection layer 430. Together, reaction area compartment 444, reaction area 414 and reaction area output 416 are in fluid communication and comprise a reaction area column. Reaction layer 420 may contain more than one reaction area column. Reaction layer 420 may also contain one or more reaction layer reaction vessels 452, arranged on first surface 421, which may be configured to contain prepackaged dry or liquid reagents.

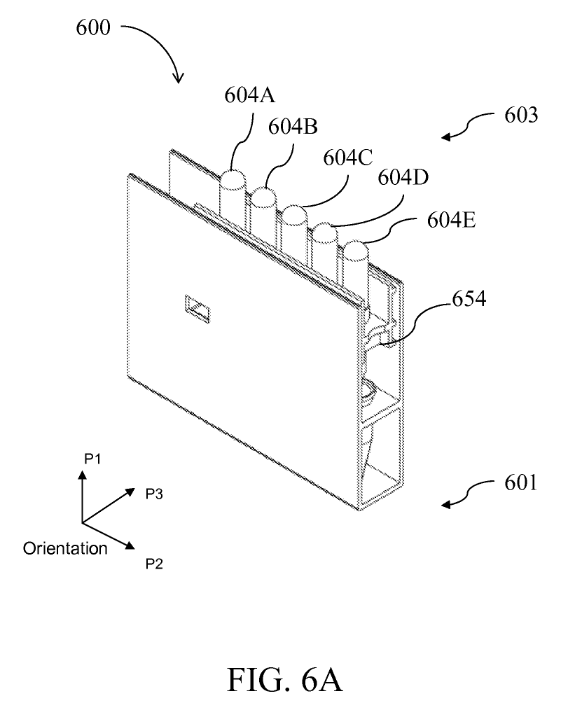

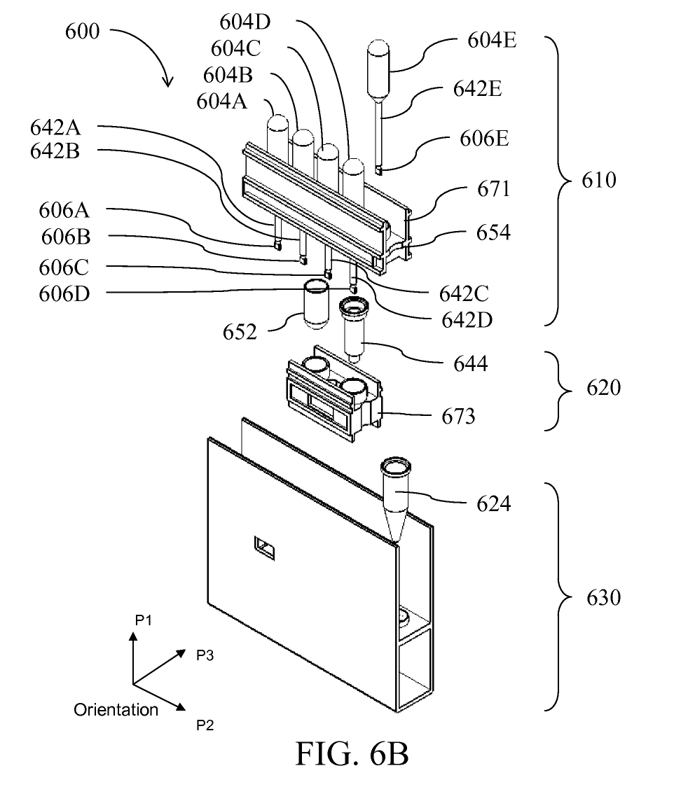

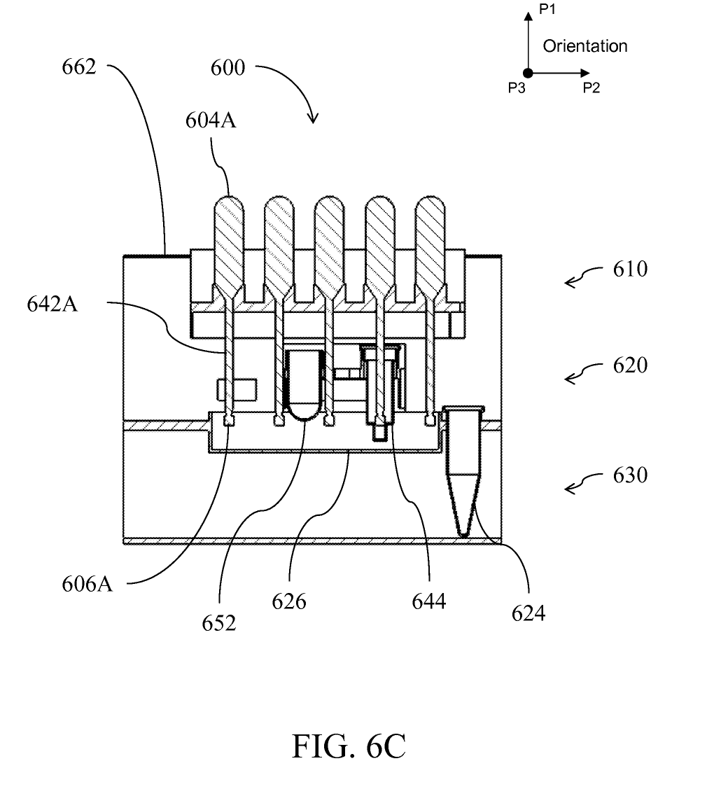

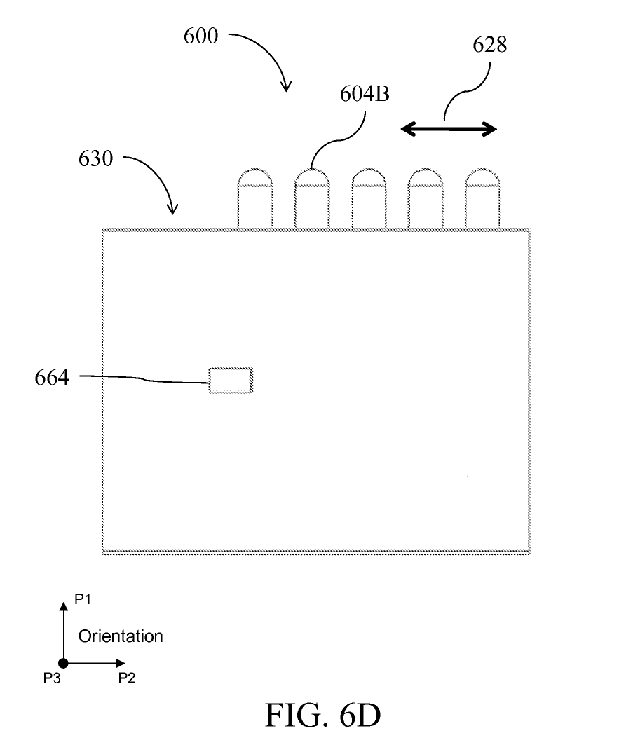

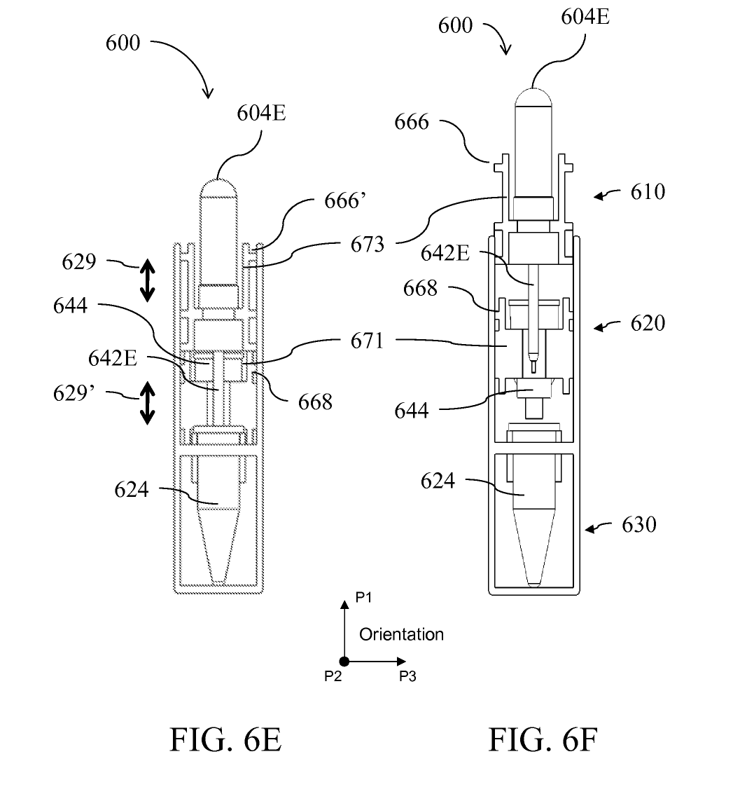

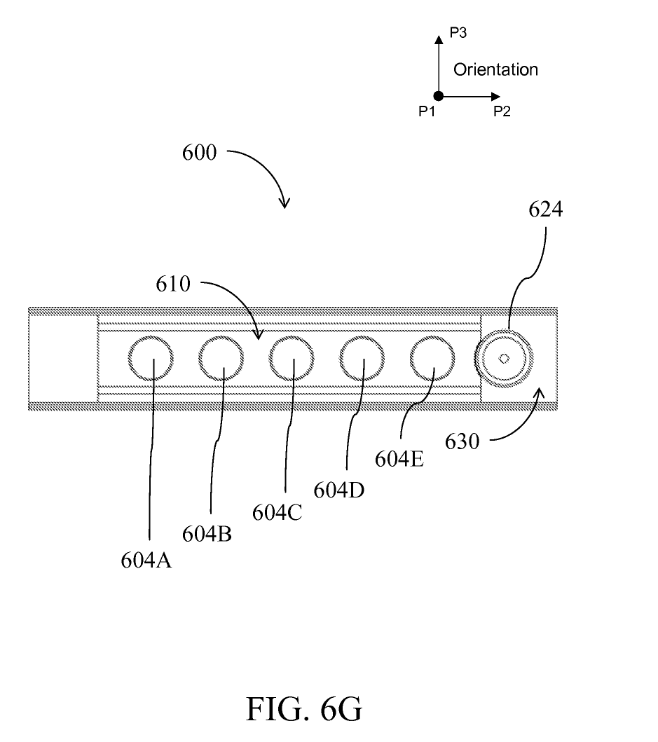

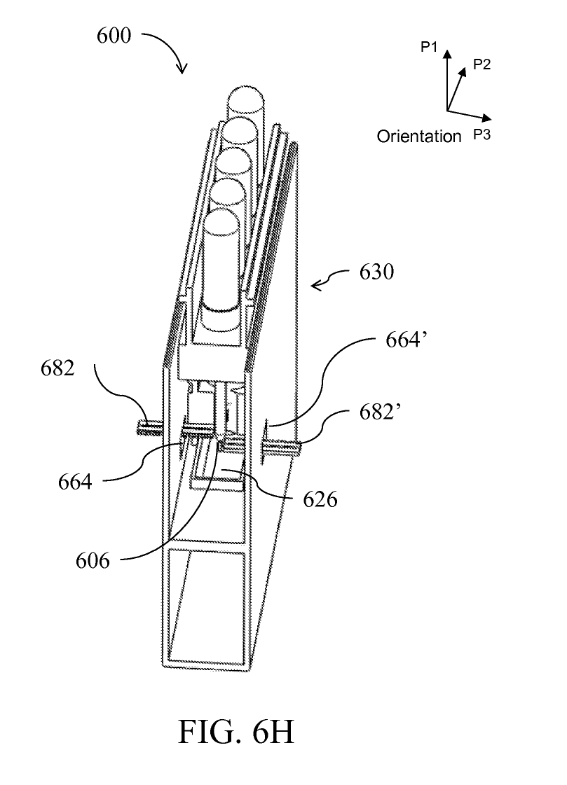

During operation, tear off frangible seals 406A, 406B, 406C, 406D are selectively openable by, for example tearing, twisting or cutting of seals 406A, 406B, 406C, 406D. This operation may be carried out by the fluid processing apparatus, for example as shown in FIGS. 6H, 6I. Alternatively, seals 406A, 406B, 406C, 406D may be pressure sensitive seals and are openable upon compression of reagent compartments 404A, 404B, 404C, 404D. Referring to FIG. 4, frangible seal 406D has been removed by the fluid processing apparatus (not shown) from tip 442D. Insertion of tip 442D into reaction area compartment 444, followed by compression of reagent compartment 404D can serve to deliver reagents stored in reagent compartment 404D into reaction area compartment 444. In a similar manner, fluids from more than one reagent compartments 404A, 404B, 404C, 404D may be added to reaction area compartment 444 or reaction vessel 452. Insertion of any reagent tips 442A, 442B, 442C, 442D into any fluids contained within the reaction area compartment 444 or reaction vessel 452, followed by compression and decompression of the reagent compartment 404 can serve to mix said fluids in these vessels. In addition to mixing, the compression and decompression of a reagent compartment 404 can serve to fill reagent compartment 404 with the mixed fluid and allow transfer of the fluids between reaction area compartment 444 and reaction vessel 452.

Reaction area compartment 444 and reaction vessel 452 may interface with fluid handling apparatus components designed to regulate the temperature of vessel contents. As an example, a reaction that could be carried out in these vessels include, but are not limited to, enzymatic treatments of a biological sample to lyse hardy microorganisms to allow access to their content. As another example, a reaction that could be carried out in these vessels include enzymatic or chemical treatments of a nucleic acid sample to shear, digest, extend, ligate, or convert the nucleic acid sample.

Air tight seals may or may not need to be created between reagent tip 442 and reaction area compartment 444. If an air tight seal is formed between reagent tip 442 and reaction area compartment 444, compression of the attached reagent compartment 404 will generate pressure which may force the fluids out through reagent tip 442, into reaction area compartment 444, through the reaction area 414 and out through reaction layer outlet 416. Passage of fluids through the reaction area 414 may result in retention of analytes in the fluid onto the reaction layer 420. Alternatively, the flow of fluids through reaction area 414 may result in the removal of bound contaminants or analytes from reaction area 414. Alternatively, reagent layer 410 in this embodiment may contain a fitting 44,8 arranged on a second surface 413, which is in fluid communication with a gaseous or reagent source 446 external to the fluid handling device 400. Air tight sealing between fitting 448 and the reaction area compartment 444 allows delivery of gases or reagents to the reaction area 414. Delivery of gases to compartment 444 may serve to drive any residual fluids in compartment 444, through reaction area 414 and reaction layer outlet 416. Fluid flow emanating from reaction layer outlet 416 can be collected into one or more fluid collection compartments (424 or 426) arranged in fluid collection layer 430. Alternatively, fittings to supply gases or reagents may be part of the operating apparatus and not part of reagent layer 410. Independent motion of the reagent layer 410, reaction layer 420 and fluid collection layer 430 allows alignment of the reaction area column to any tip in reagent layer 410 and allows alignment of the reaction area output 416 to any fluid collection compartments 424 or 426 in the fluid collection layer 430. In this exemplary embodiment, alignment of the various compartments, inlets, outlets, tip, etc. is accomplished by linear motion 428 along an axis P2 and vertical motion 429 along an axis P1 rather than rotary motion about an axis as provided for in the embodiment shown in FIG. 3.

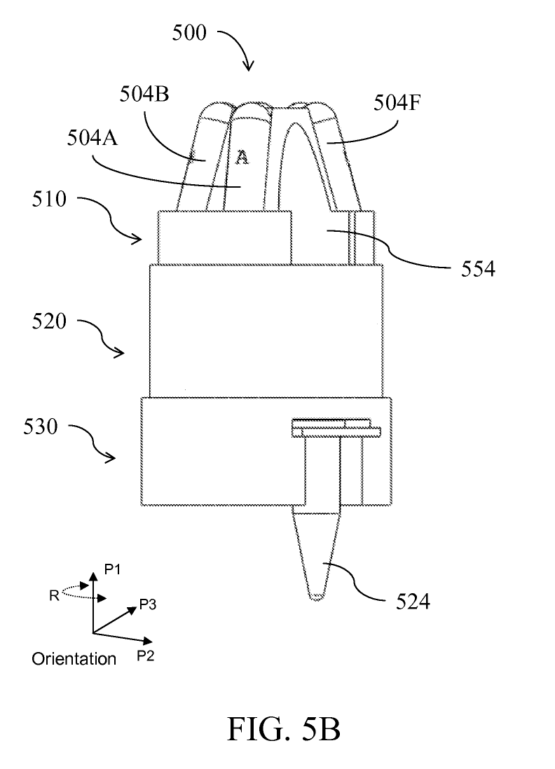

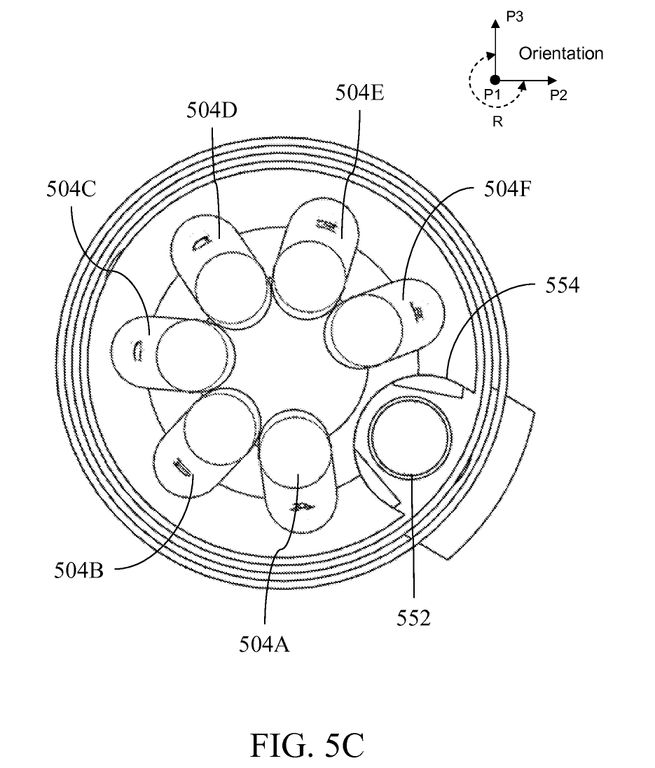

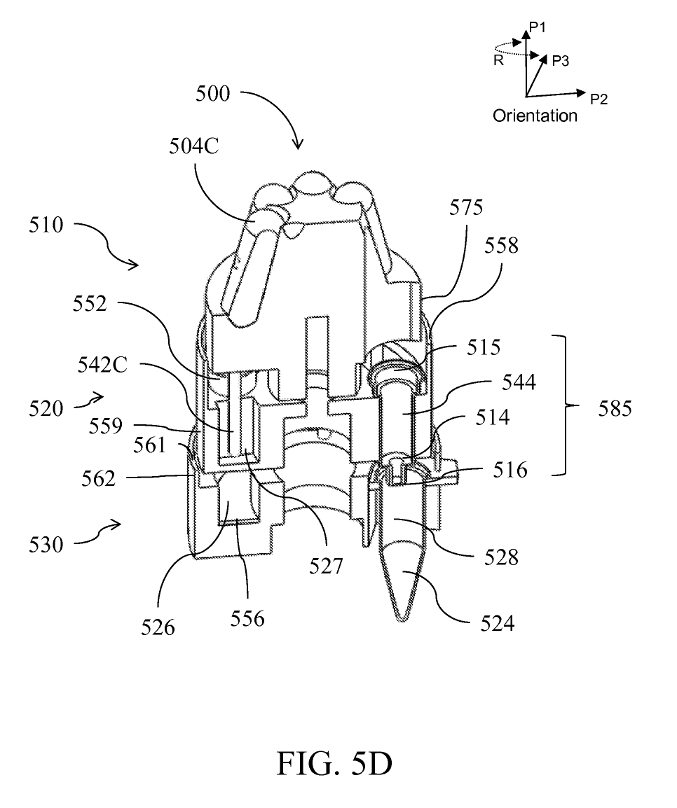

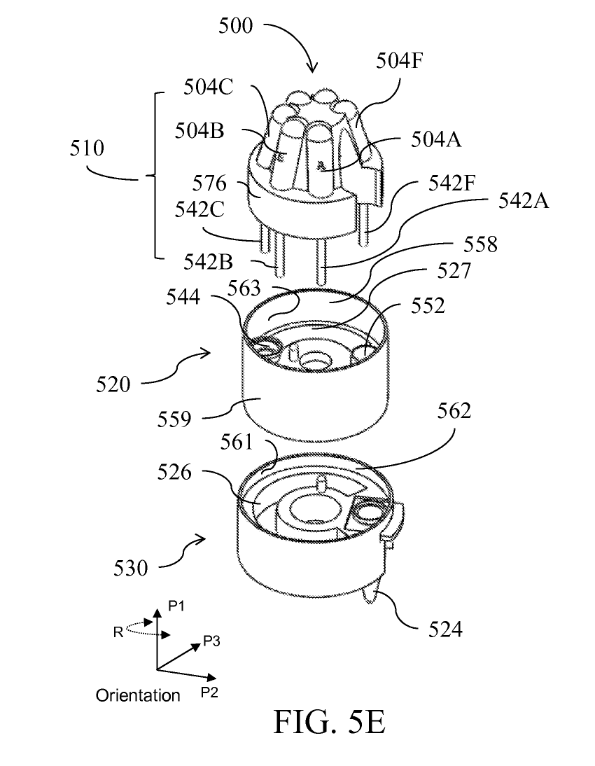

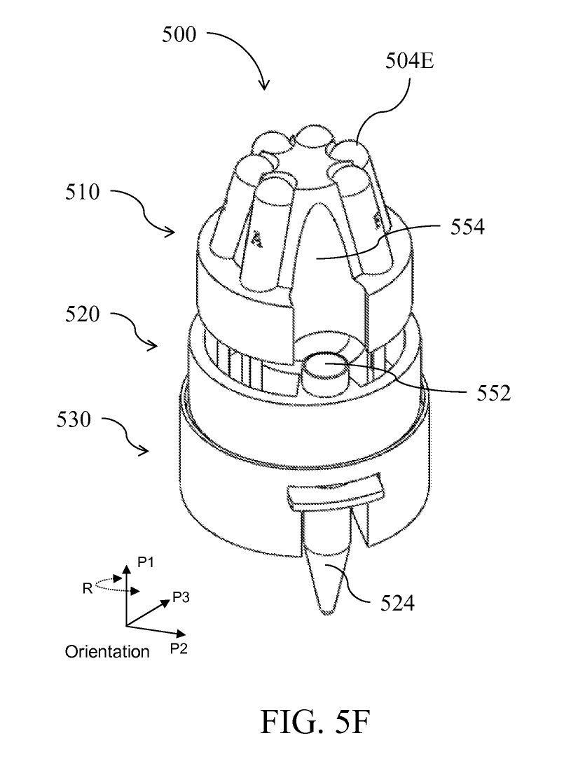

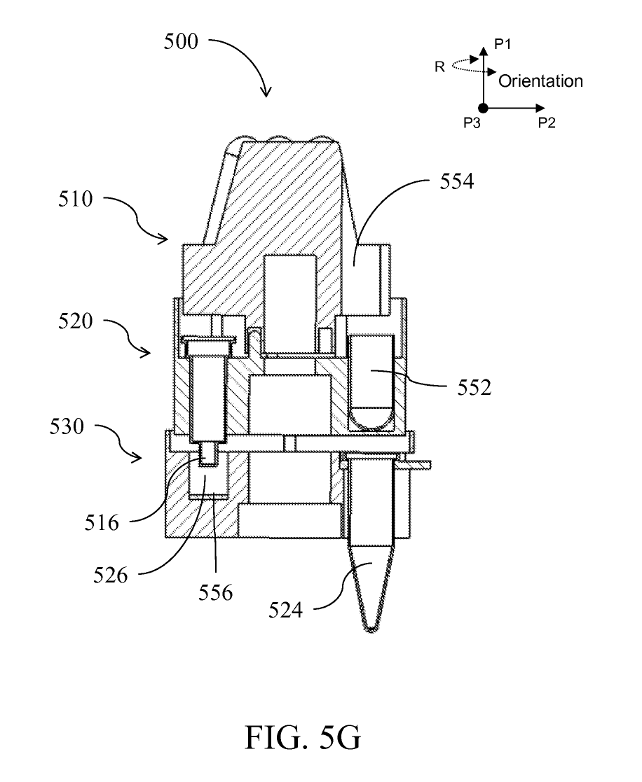

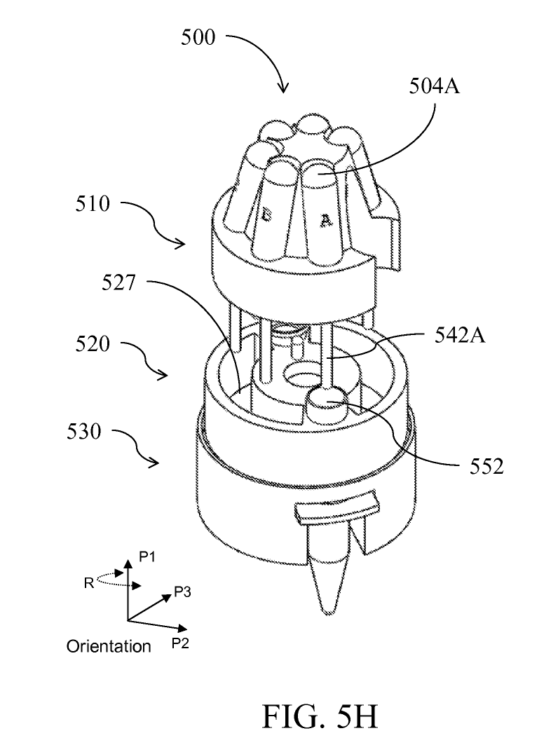

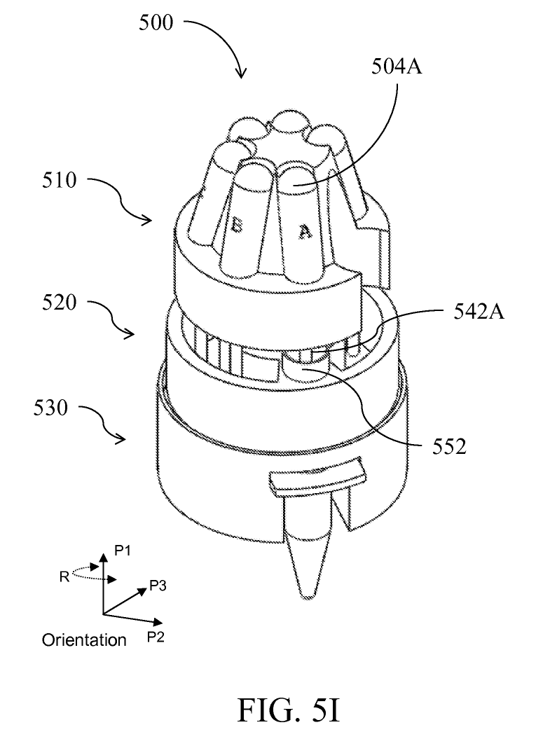

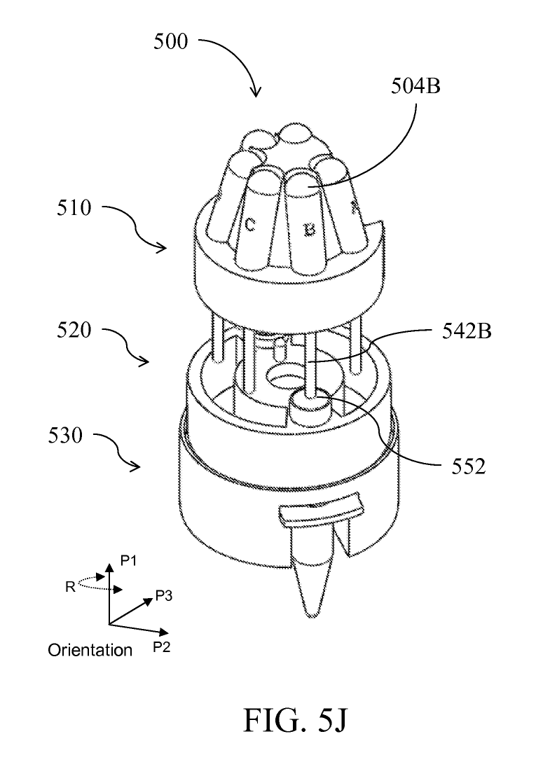

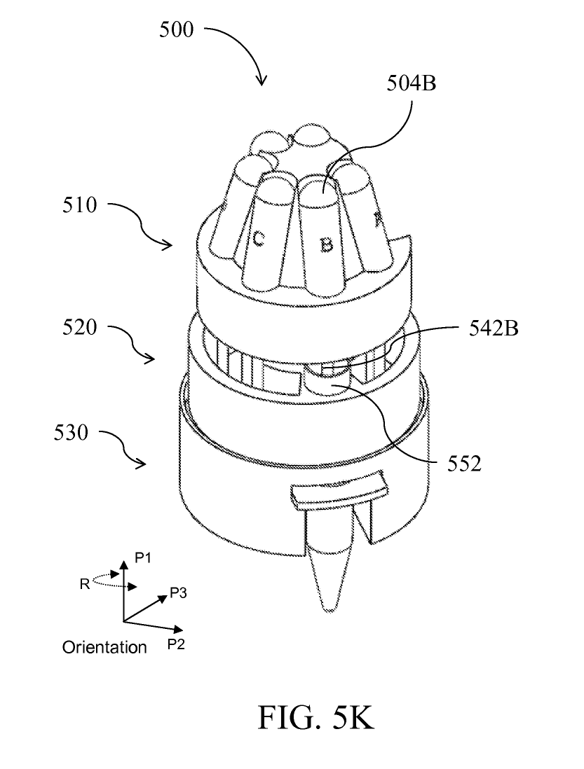

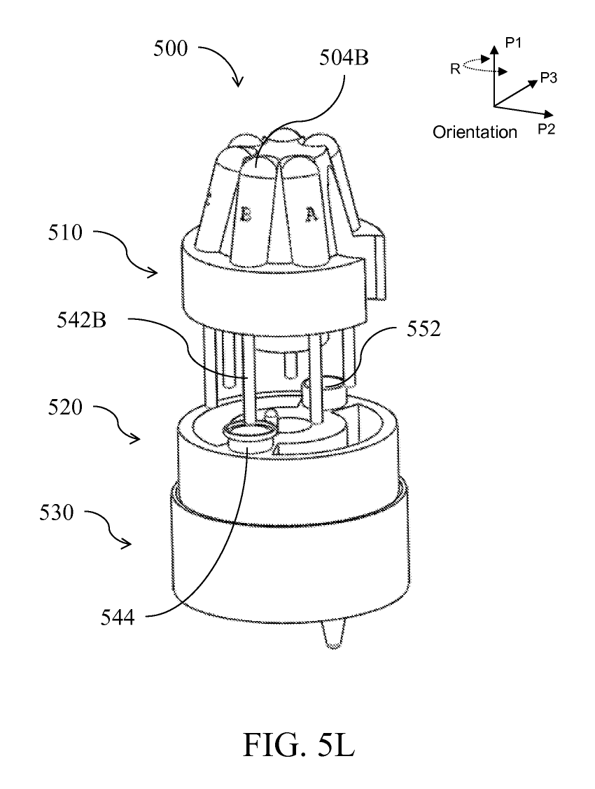

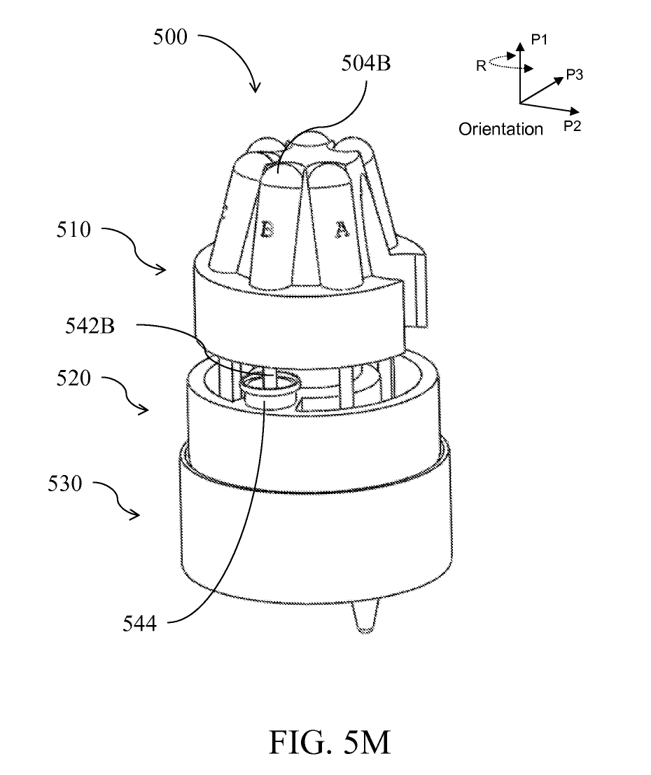

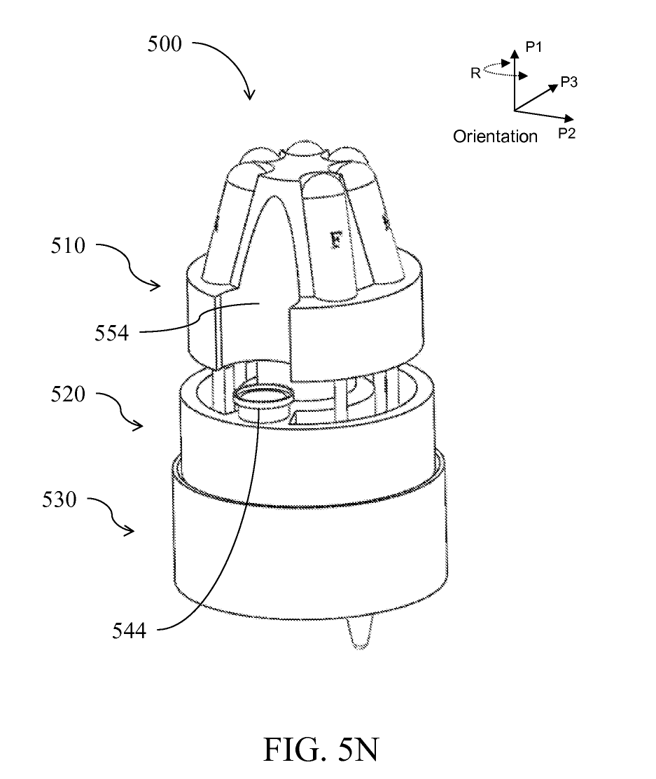

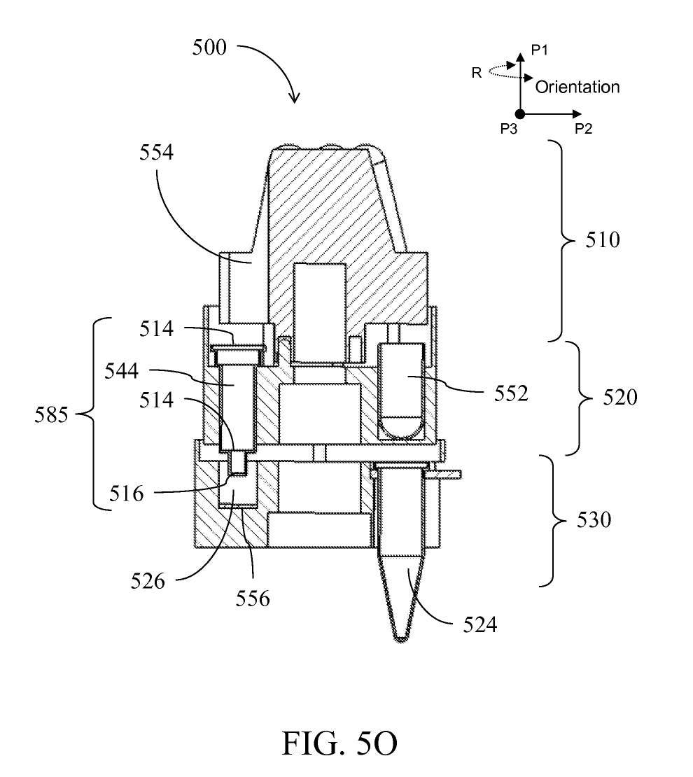

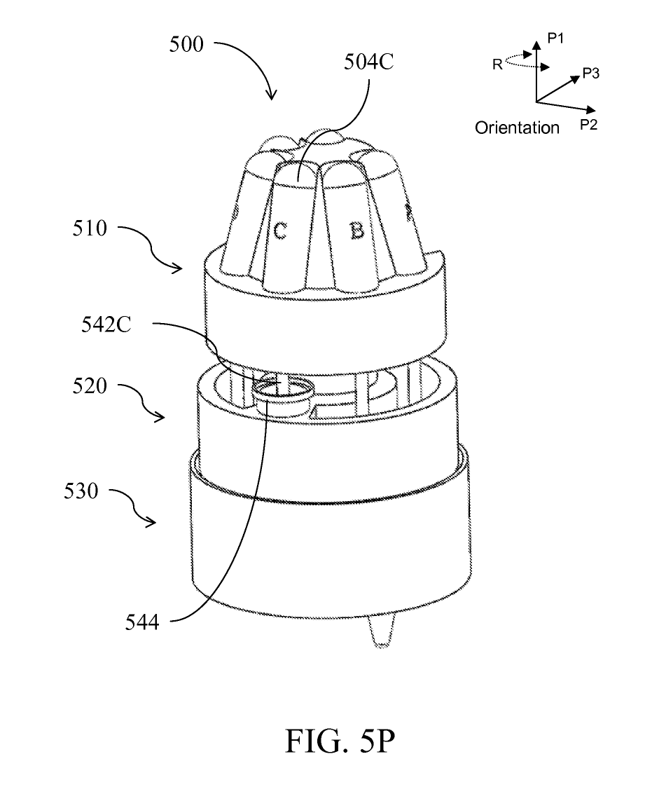

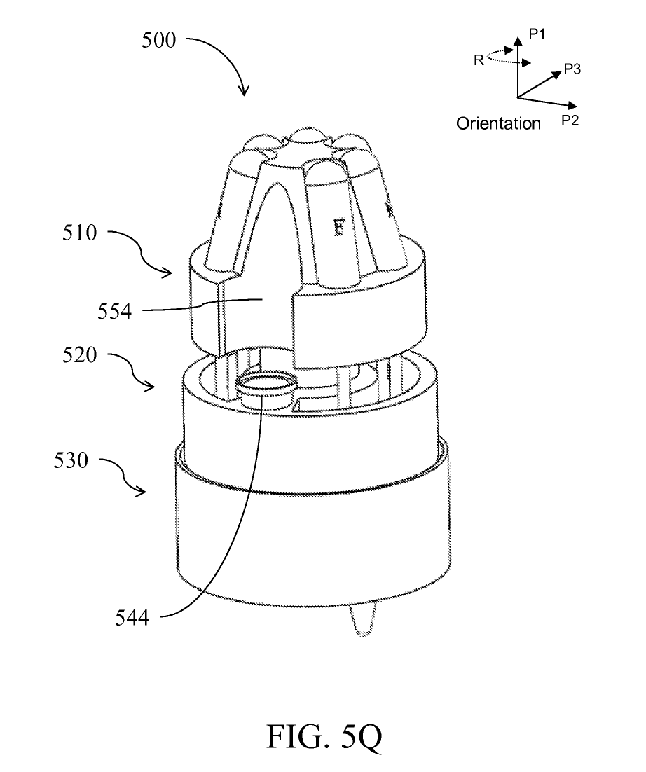

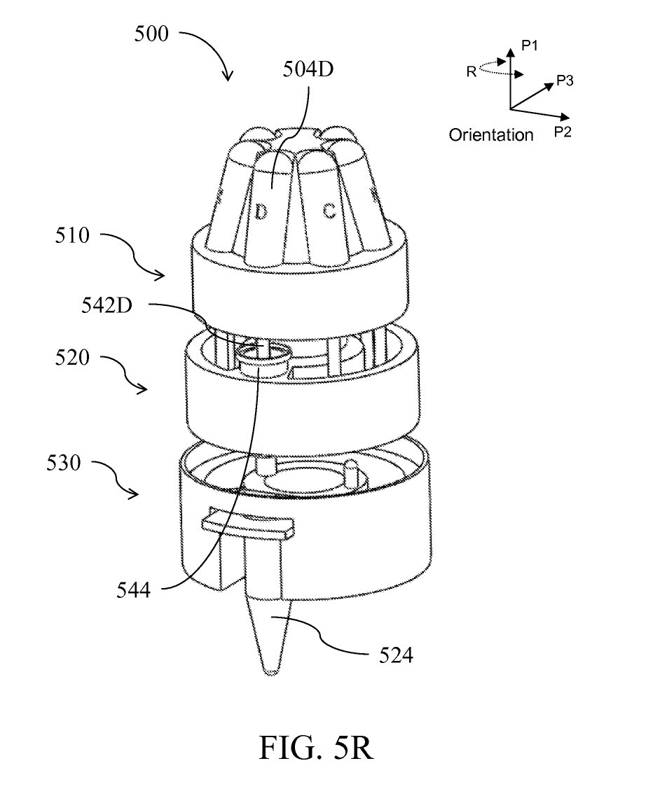

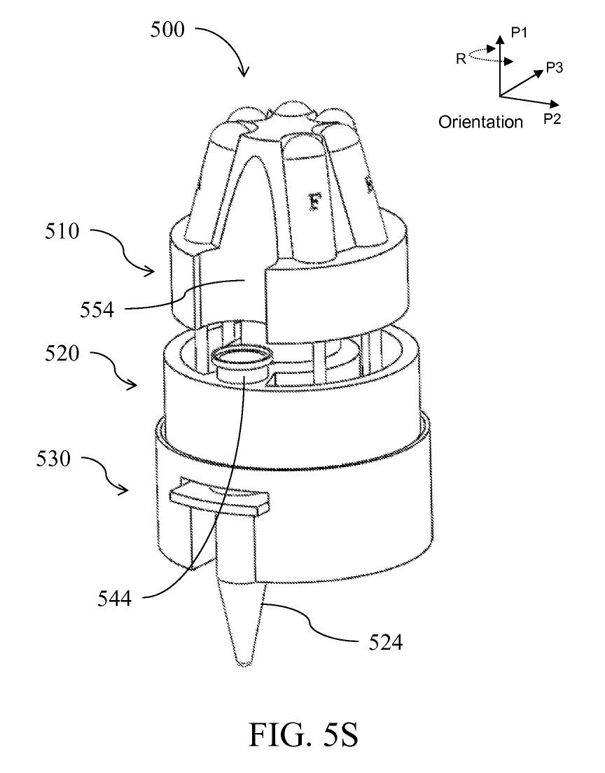

FIG. 5A shows a 3-dimensional representation of another fluid handling device 500 which incorporates many of the features shown in previous figures. The fluid handling device has a circular cross section in a first plane (P2-P3) and a substantially rectangular cross section in a second and third plane (P1-P2/P1-P3). A first end 501 can be configured to provide a planar surface capable of being set on, for example, a table without falling over and a second end 503 forming a somewhat conical shape. This view shows reagent layer 510, reaction layer 520 and fluid collection layer 530 nested together. Reagent layer 510 contains the reagent compartments 504A, 504B, 504C, 504D, 504E, 504F and a cutout 554 to allow access of fittings from the fluid processing apparatus to supply gases or reagents. Reagent compartments 504A, 504B, 504C, 504D, 504E, 504F can be integral to reagent layer 510 or be a separate subcomponent which is attachable to reagent layer 510. Fluid collection layer 530 contains the waste collection compartment 526 (not visible in this view) and an eluate collection compartment 524, which, in this example, takes the form of a common test tube. Eluate collection compartment 524 can be integral to layer 530 or be a separate subcomponent which is attachable to layer 530. Fluid handling devices can be formed such that each layer is integrally formed, one or more layers is integrally formed with at least one other layer and so on. Integrally formed components can be configured such that each component or feature that is essential or necessary for completeness is provided. Moreover, the components can be constructed such that they have a unitary construction or such that they act in a unified manner once formed. Eluate collection compartment 524 can contain prepackageable reagents such as lyophilized buffers, nucleic acids and enzymes. Eluate collection compartment 524 is coverable by a lidding layer, such as aluminum lidding for storage and transport. Other suitable mechanisms to cover or seal eluate collection compartment 524 can be used without departing from the scope of the invention.

FIG. 5B shows a side view of device 500. FIG. 5C shows the top view in plane P2-P3 of device 500. Reagent compartments 504A, 504B, 504C, 504D, 504E, 504F and cutout 554 are arranged axially around the first surface of reagent layer 510. A reaction vessel 552 can be seen through cut out 554.

FIG. 5D shows a section through, for example, a vertical plane, P1-P2, of the fluid handling device 500. Fluid collection layer 530 and reaction layer 520 contain collection layer side wall 562 and reaction layer side wall 558 which defines (as shown in cross-section) an exterior border in the P2-P3 plane of reaction layer 520 and reagent layer 510 respectively. Each side wall has an interior surface and an exterior surface. These side walls 562, 558 are configured such that at least a portion of, for example, an exterior surface 559 of the reaction layer side wall 558 fits within and abuts an interior surface 561 of the fluid collection layer side wall 562 which can serve to reduce the escape of aerosols or fluids during operation of the fluid handling device. As will be appreciated by those skilled in the art, the opposing configuration can be employed without departing from the scope of the disclosure such that the interior surface of the reaction layer side wall mates with and abuts an exterior surface of the fluid collection layer side wall. Similarly the reagent compartment has a side wall 575 which has an exterior surface and which is configured such that at least a portion of the exterior 576 of reagent layer side wall 575 fits within and abuts an interior surface 563 of solid layer side wall 558. The reaction layer 520 contains a reaction area column 585 as depicted here includes the combination of reaction area compartment 544, reaction area filter 514 and reaction area outlet 516. Other components can be included within a reaction area column 585 without departing from the scope of the invention.

Reaction area compartment 544 has an inlet 515 which faces upward in communication with the bottom of reagent layer 510. Reaction area outlet 516 opens (as shown, downward) in communication with the first side of fluid collection layer 530. Reaction area compartment 544, reaction area filter 514 and reaction area outlet 516 are in fluid communication and together comprise a reaction area column. The reaction area column can be formed such that it is integral to reaction layer 520 or form a separate subcomponent which is attachable to reagent layer 520. Integrally formed components can be configured such that each component or feature that is essential or necessary for completeness is provided. Moreover, the components can be constructed such that they have a unitary construction or such that they act in a unified manner once formed. As will be appreciated by those skilled in the art, the form factor of the reaction area column depicted here is similar to commonly used spin filters for protein or nucleic acids purification but can be of other form factors.

In FIG. 5D, the reaction area column outlet 516 is positioned in the eluate collection inlet 528 of the eluate collection compartment 524 and places the reaction area column in fluid communication with the eluate collection compartment 524. If eluate collection compartment 524 is sealed with a lidding layer (not shown), such as aluminum lidding for storage and transport, such lidding layers are easily pierced by the reaction area column outlet 516 during operation of device 500. The one or more reagent compartments 504A, 504B, 504C, 504D, 504E, 504F contain one or more associated tips, 542A, 542B, 542C, 542D, 542E, 542F (more clearly seen in FIG. 5E) and are similar to common, plastic “transfer pipettes” or a unit dose dispensing device as described in U.S. Pat. Nos. 6,869,419 and 6,652,494 and European Patent EP 1086661. The reagent compartments 504A, 504B, 504C, 504D, 504E, 504F are in fluid communication with their associated tips, 542A, 542B, 542C, 542D, 542E, 542F. These pipettes are Tillable with reagents of choice and sealable at the tips for long term storage. In FIG. 5D, a reaction vessel 552 can just be seen positioned behind one of the reagent compartment tip 542C. As with the reaction area column (as represented by the combination of compartment vessel 544, reaction area 514 and output 516), the reaction vessel 552 can be integral to layer 520 or be a separate subcomponent which is attachable to layer 520. Reaction vessel 552 can contain prepackageable reagents such as lytic enzymes and is coverable by a lidding layer, such as aluminum lidding for storage and transport. Such lidding layers are easily pierced by the user with a manual pipette tip during reagent or sample introduction into reaction vessel 552 or by tips 542A, 542B, 542C, 542D, 542E, 542F on the reagent layer 510 during operation of device 500. Other suitable mechanisms to cover or seal reaction vessel 552 can be used without departing from the scope of the invention. The body of layer 510 sits on top of this vessel and can act as a lid to minimize evaporation, for example, during enzymatic incubation.

FIG. 5E shows a trough 527 in layer 520 which opens upward in communication with the bottom of reaction layer 510. This trough serves to accept reagent compartment tips 542A, 542B, 542C, 542D, 542E, 542F when they are not positioned either in the reaction vessel compartment 552 or in the reaction area column compartment 544. An example of a compartment tip resting in trough 527 can be more clearly seen in FIG. 5D where compartment tip 542C can be seen in trough 527. If reagent compartment tips 542A, 542B, 542C, 542D, 542E, 542F contain twist off or break-away seals, one or more troughs 527 can serve as a waste compartment to accept these seals as they are removed from tips 542A, 542B, 542C, 542D, 542E, 542F during use of the device. Fluid collection layer 530 contains one or more waste compartments 526. A suitable waste compartment 526 is a trough running nearly the circumference of layer 530. Waste compartment 526 opens on its first surface (depicted as upward) in communication with the second surface (depicted as bottom) of reaction layer 520. This trough may contain absorbent materials 556 (see FIG. 5D) to absorb any waste fluids emanating from outlet 516 and/or agents suitable to neutralize or react with any waste fluid emanating from outlet 516. In this manner, waste fluids are absorbable to minimize contamination risks such as may occur when the user disposes of the device after use. Additionally, in some configurations, more than one waste compartment may be provided where it is desirable for waste from various steps to be segregated after processing.

FIGS. 5F-S describe how device 500 can be used to execute the process shown in FIG. 1. FIGS. 5F-G show device 500 in its starting configuration. In this view, the side wall 558 on the reaction layer 520 has been drawn down to allow better views of the inner workings of the device. Here reaction compartment 552 is positioned under cutout 554 to allow introduction of samples by the user into compartment 552. FIG. 5G shows a cross section side view of this initial configuration through the plane P1-P2. FIGS. 5H-I show the introduction of lysis buffer into the sample. The reagent layer 510 is moved (e.g., raised) along axis P1 and rotated R around axis P1 such that reagent compartment 504A and its associated tip 542A is aligned with a target reaction compartment, such as reaction compartment 552. In this figure, the lysis buffer is storable in reagent compartment 504A. If the reagent compartment tips are sealed with twist off or breakaway seals, these seals are first removed and are dropped into waste trough 527, prior to alignment with compartment 552. The lysis buffer reagent compartment tip 542A is lowered along axis P1 into the reaction compartment 552 placing compartment tip 542A in fluid communication with reaction compartment 552 as shown in FIG. 5I. Alternate cycles of compression and decompression of the reagent compartment bulb 504A in layer 510 by the fluid processing apparatus will mix the lysis buffer with the sample.

If an incubation step is required, the fluid processing apparatus can provide thermal control to compartment 552 (not shown). In a similar manner, a binding buffer storable in reagent compartment 504B can be delivered and mixed with the lysate as shown in FIGS. 5J-K. This can be achieved by, displacement of reagent layer 510 along axis P1 in a first direction (e.g., upward), followed by rotation R around axis P1, followed by displacement of reagent layer 510 along axis P1 in a second direction (e.g., downward), followed by alternate cycles of compression and decompression of the reagent compartment. After mixing, the resultant mixture can be aspirated back up into the reagent compartment 504B by compression and decompression of reagent compartment 504B. Displacement of reagent layer 510 along axis P1 followed by rotation R of layer 510 (or layer 520) around axis P1, can now align reagent compartment 504B with the reaction area column compartment 544 (FIG. 5L) and the mixture delivered into compartment 544 (FIG. 5M) in a manner similar to the process described above. The combination of displacement along axis P1 in one or more directions and rotation R around axis P1 of reagent layer 510 is again used to align cutout 554 with the reaction area column compartment 544 (FIG. 5N). FIG. 5O shows a side view cross section along plane P1-P2 of the device configuration shown in FIG. 5N. Cutout 554 allows access to a fitting from the fluid processing apparatus (not shown) to make an air tight seal with reaction area column compartment 544. The fluid processing apparatus can deliver gaseous pressure to force the fluid in reaction area column compartment 544 though the reaction area 514, and out through the outlet 516. In this step, nucleic acids are specifically bound to the reaction area.

In FIG. 5O reaction area outlet 516 is aligned over waste compartment 526 of fluid collection layer 530 placing the reaction area outlet 516 in fluid communication with waste compartment 526. The resultant waste fluid from the reaction area column 585 is collectable in waste compartment 526 and absorbed by material 556. The reaction area is washable by buffers contained in subsequent reagent compartments 504. FIG. 5P shows an alignment of one such wash reagent compartment (marked as 504C) with the reaction area column compartment 544. Vertical and rotational alignments needed to carry out this step are similar to steps described previously. Wash fluids delivered to reaction area column compartment 544 can be forced through the filter and out to the waste compartment 526 by gaseous pressure delivered by the fluid processing apparatus (not shown). Access to reaction area column compartment 544 is provided by the reaction area column compartment cutout 554 as shown in FIG. 5Q. Multiple wash steps can be executed in this manner. Nucleic acids bound to the reaction area can be eluted by delivery of elution buffer stored in reagent compartment 504D (shown in FIG. 5R) into reaction area column compartment 544. FIG. 5R also shows rotation of layer 530 such that the collection compartment 524 is aligned under the reaction area column placing eluate collection compartment 524 in fluid communication with the reaction area column. FIG. 5S shows the cutout aligned over reaction area column compartment 544. Gaseous pressure from the fluid processing apparatus can now be delivered to the reaction area column via reaction area column compartment 544 and the nucleic acids is released from the reaction area and delivered to the collection compartment 524. Reagent compartments 504E and 504F are not in use during this example process, but may contain additional wash reagents or reagents optimized for other fluid handling processes such as lysis buffers, binding buffers, wash buffers, elution buffers, reaction buffers, dilution buffers, aqueous solutions, organic solutions, protein solutions, and dried reagents.

FIGS. 6A-I show a 3-dimensional representation of alternative fluid handling device 600 with a linear form factor, which incorporates many of the features shown and described with respect to the previous figures. One primary difference in this device is the use of linear and vertical motion instead of rotary and vertical motion to position the layers and components in a targeted alignment prior to executing a particular process. FIG. 6A shows an isometric view of the assembled device 600 which has rectangular cross-section in planes P1-P2, P2-P3 and P1-P3. A first (bottom) end 601 can be configured to provide a planar surface such that it is capable of being set on, for example, a table or other surface without falling over and a second (top) end 603 that is open. One or more reagent compartments are provided for, as shown reagent compartments 604A, 604B, 604C, 604D, 604E and cutout 654 are arranged in a linear row, e.g. along axis P2, within the second end with the body of the reagent compartments 604A, 604B, 604C, 604D, 604E protruding out of, and accessible from, an open second end of fluid handling device 600.

The exploded view of fluid handling device 600 in FIG. 6B shows three nested layers: a reagent layer 610, a reaction layer 620 and a fluid collection layer 630. Reagent layer 610 contains a reagent layer body 671 which is adapted and configured to hold one or more reagent compartments 604A, 604B, 604C, 604D, 604E. The one or more reagent compartments 604A, 604B, 604C, 604D, 604E are configurable to be in fluid communication with a corresponding one or more tip 642A, 642B, 642C, 642D, 642E (pointing downward toward reaction layer 620) and one or more breakaway seals 606A, 606B, 606C, 606D, 606E arranged in a linear row. Reagent layer body can further be configured to provide a cutout 654 arranged next to reagent compartment 604E to allows access of fittings from the fluid processing apparatus (not shown) to supply gases or reagents. Reagent compartment 604E with its associated tip 642E and breakaway seal 606E are shown as detachable from reagent layer 610.

The reaction layer 620 has a reaction layer body 673 which holds reaction vessel 652, and reaction area column 644. The form factor of the reaction area column depicted here is similar to commonly used spin filters for protein or nucleic acids purification but can be of other form factors. Reaction vessel 652 and reaction area column 644 are open at the top and are in communication with the reagent compartment tips 642A, 642B, 642C, 642D, 642E. Reaction vessel 652 and reaction area column 644 are shown as detachable from reaction layer body 673.

Fluid collection layer 630 contains the waste collection compartment 626 (better shown in FIG. 6C) and an eluate collection compartment 624, which, in this example, takes the form of a common test tube. Waste collection compartment 626 and eluate collection compartment 624 are open at the top and are in communication with the bottom of reaction area column 644. Eluate collection compartment 624 is shown as detachable from fluid collection layer 630. The detachability of the reagent compartments 604A, 604B, 604C, 604D, 604E (with their associated tip and seals), the reaction vessel 652, reaction area column 644, and eluate collection vessel 624 allows customization of the reagents that are stored in reagent layer 610, reaction layer 620, and 630.

The cross-sectional view of device 600 in FIG. 6C shows the relative positions of reagent compartments 604A with tip 642A and breakaway seals 606A, reaction vessel compartment 652, reaction area column compartment 644, waste compartment 626 and eluate collection compartment 624. Fluid collection layer 630 includes side walls 662 which overlap reaction layer 620 and reagent layer 610. This overlap serves to reduce the escape of aerosols or fluids during device operation.

FIG. 6D shows a side view of fluid handling device 600 in the plane P1-P2 indicating the relative horizontal motion 628 of reagent layer 610, reaction layer 620 and fluid collection layer 630. Reagent layer 610 and reaction layer 620 (not visible in this figure) move horizontally within fluid collection layer 630. FIGS. 6E-F show end views of the fluid handling device 600 in the plane P1-P3. Reagent layer 610 can move vertically, 629, along axis P1 and reaction layer 620 can move vertically, 629′, along axis P1. FIGS. 6E-F show rails/stops 666 on reagent layer body 673 and rails/stops 668 on reaction layer body 671 that act to simplify the motion control for the device. They provide both motion limits and sliding surfaces so that reagent layer 610, reaction layer 620 and fluid collection layer 630 can be moved relative to each other. FIG. 6F shows how reagent layer 610 (with reagent compartments 604E and tips 642E visible) and reaction layer 620 (with reaction area column 644) have been raised to new positions. Layer 630 (with eluate collection compartment 624) remains in the same position in this example. The combination of horizontal and vertical motion position the reagent layer 610, reaction layer 620 and fluid collection layer 630 in proper locations to execute the process. Not shown in FIGS. 6D-F are the connection points with the fluid processing apparatus that provides the motion. These connection points may be holes or slots in reagent layer 610, reaction layer 620 and fluid collection layer 630 to allow the layers to be moved to new positions or held stationary in the same position. FIG. 6G shows a top view of device 600. Reagent compartments 604A, 604B, 604C, 604D, 604E are shown visible in reagent layer 610 and eluate collection compartment 624 is shown visible in fluid collection layer 630.

FIGS. 6H-I show one exemplary method of removing the reagent compartment seals 606. FIG. 6H is a side view of fluid handling device 600 and FIG. 6I is a top view of fluid handling device 600. A set of serrated teeth 682 and 682′ are moved into fluid handling device 600 through openings 664 and 664′ in fluid collection layer 630. The motion twists the seals 606 which, upon separation, are collected in waste container 626. Reagent compartment seal removal devices 682 and 682′ may alternatively provide cutting instead of twisting action. Other suitable methods for removing the reagent compartment seals 606 can be used without departing from the scope of the invention.

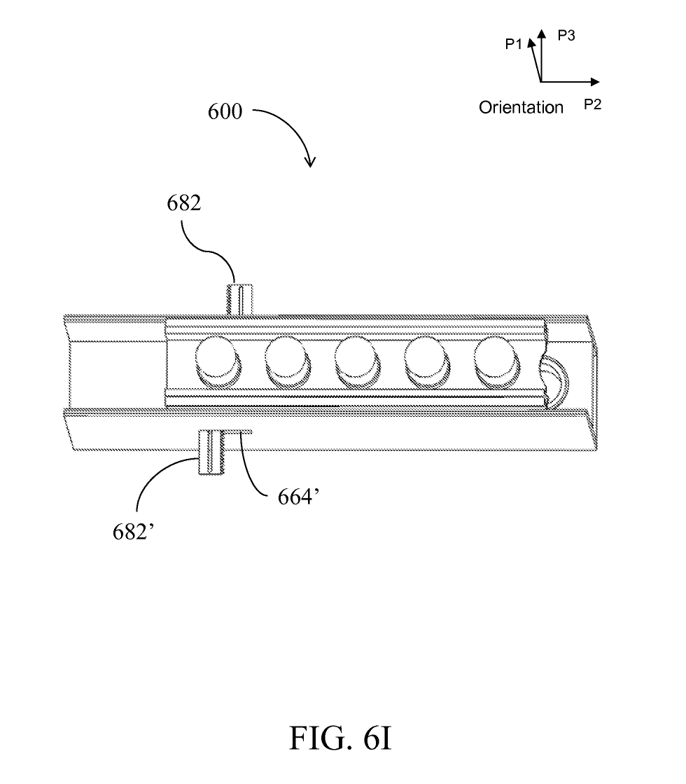

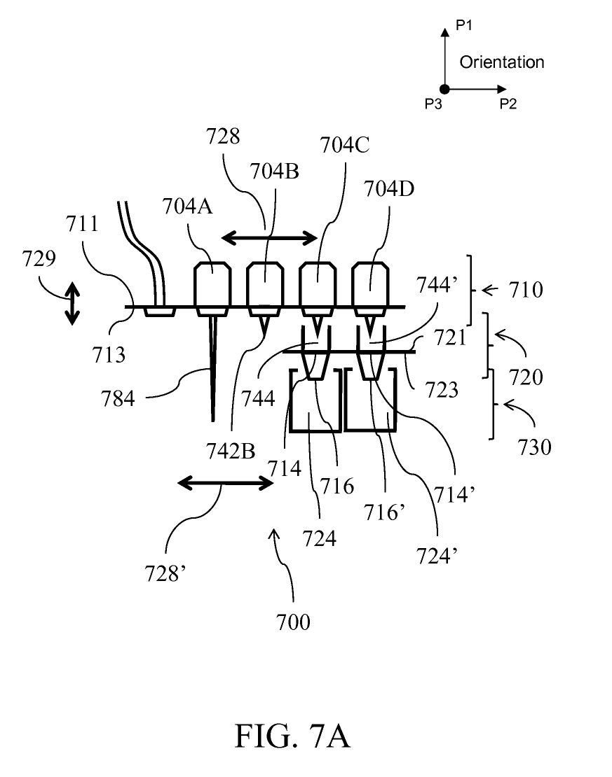

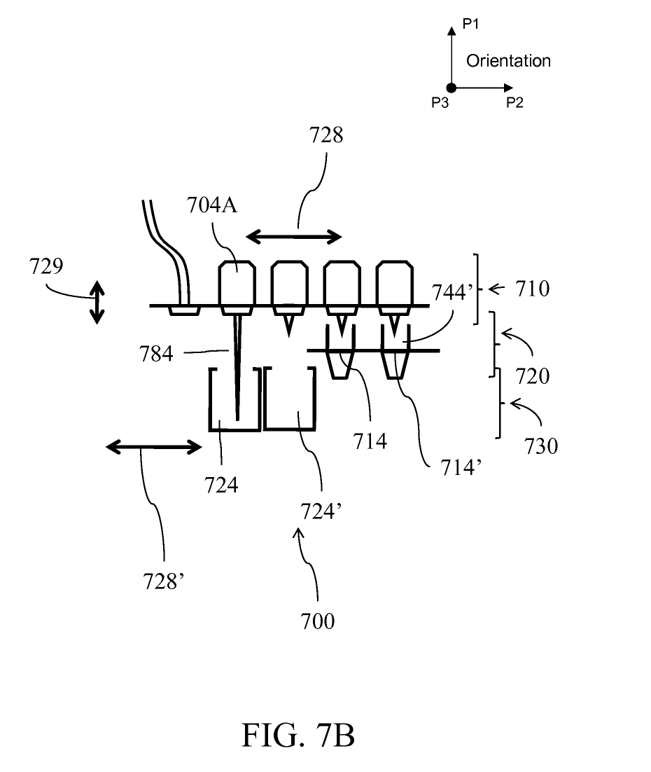

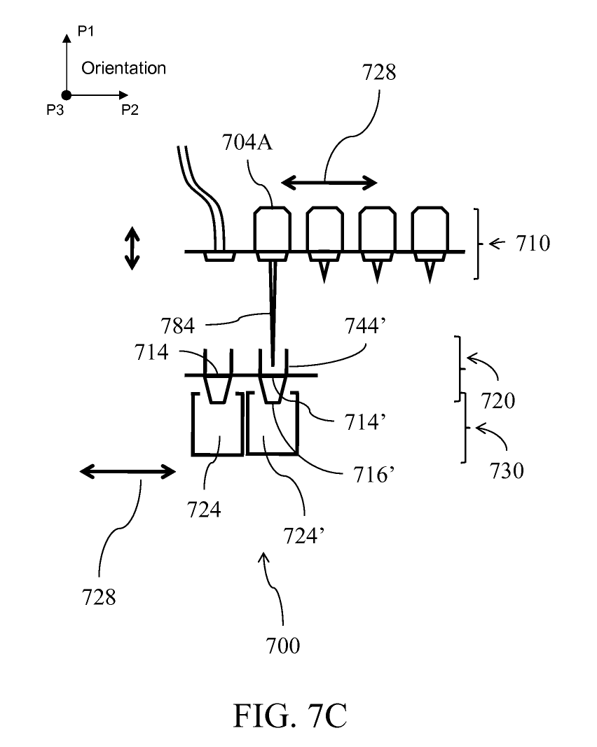

FIG. 7A shows another fluid handling device 700, which contains reagent layer 710, reaction layer 720, and fluid collection layer 730. Reagent layer 710 contains one or more reagent compartments 704A, 704B, 704C, 704D, residing on, for example, a first surface 711. Reagent compartments 704B, 704C, 704D are in fluid communication with one or more corresponding tips 742B, 742C, 742D, which extends from, for example, a second surface 713 (depicted as downward) of reagent layer 710. For clarity, of the plurality of tips 742B, 742C, 742D, that may be provided only tip 742B is labeled in FIG. 7A.

Reagent compartment 704A is in fluid communication with an extra long tip, 784. All of the tips, 742B, 742C, 742D, 784 may have frangible seals at their tips (not shown). Reaction layer 720 has a first surface 721 and an opposing second surface 723 arranged below reagent layer 710 in communication with an opposing second surface 713 of reagent layer 710. Reaction layer 720 contains reaction area compartment 744, reaction area 714, and reaction area output 716. Reaction area compartment 744, arranged on first surface 721, is open at the top and is in communication with an opposing second surface 713 of reagent layer 710. Reaction area output 716, arranged on second surface 723 of the reaction area 720, is open at the bottom and is in communication with fluid collection layer 730. Together, reaction area compartment 744, reaction area 714 and reaction area output 716 are in fluid communication and comprise a reaction area column. Reaction area output 716 can be placed in fluid communication with fluid collection compartment 724 in fluid collection layer 730 allowing fluid collection compartment 724 to receive fluids from 716. In some configurations, reaction layer 720 can be configured to contain a second reaction area column comprising of reaction area compartment 744′, reaction area 714′, and reaction area output 716′. Second reaction area output 716′ can be placed in fluid communication with fluid collection compartment 724′ in fluid collection layer 730 allowing fluid collection compartment 724′ to receive fluids from 716′. Independent horizontal (728 and 728′) and vertical (729) motions of the layers along axes P1 and P2 can place tip 784 into fluid communication with compartment 724 (FIG. 7B) and allows uptake of any fluids in 724 (by compression and decompression of reagent compartment 704A) up through tip 784 and into reagent compartment 704A. Independent horizontal (728 and 728′) and vertical (729) motions of the layers along axes P1 and P2 can now place tip 784 into fluid communication with compartment 744′ (FIG. 7C). Compression of reagent compartment 704A can deliver the content of reagent compartment 704A into reaction area compartment 744′. In this manner, the fluid output of column 714 can be transferred for processing through column 714′.

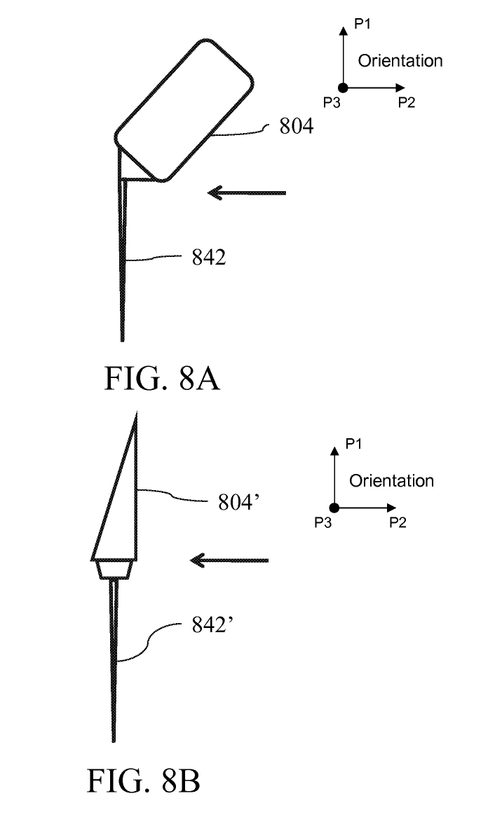

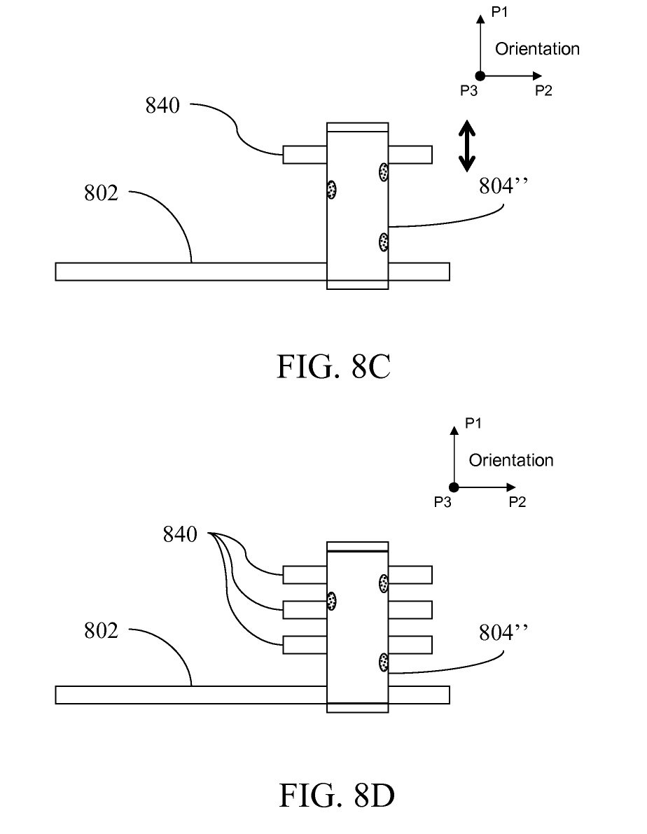

FIGS. 8A-B show alternative shapes of reagent compartments 804 and 804′. Reagent compartment 804 positioned such that it is tilted along its longest axis away from the vertical axis P1. Alternatively, reagent compartment 804′ can be configured such that it has an exterior wall that is sloped away from the vertical axis P1. Reagent compartment 804 is in fluid communication with a tip 842, which points downward and is aligned with the axis P1. Reagent compartment 804′ is in fluid communication with a tip 842′, which points downward and is aligned with the axis P1. When reagent compartment 804 or 804′ with their associated tips are attached to a reagent layer with a circular form factor, such as reagent layer 510 in the fluid handling device 500, rotation of the reagent layer will generate centrifugal force as denoted by the arrow in FIGS. 8A. This centrifugal force will force any liquid in these compartments outward and downward toward tips 842 and 842′. Tips 842 and 842′ are narrow so as to trap as little liquid or air as possible. In this manner, the majority of reagent is containable in the body of compartment 804 and 804′, toward tips 842 and 842′ and can be dispensed in a more accurate and reproducible manner.

Reagents stored in compartments 804″ may become randomly distributed within said compartments during transport. It is desirable that these reagents be collected toward the bottom of the compartments 804″ prior to device operation. FIGS. 8C-D show modules in the fluid processing apparatus that helps move droplets from the sides of the fluid compartment 804″ to the bottom of compartment 804″. These modules can be ultrasonic or other devices that generate vibratory motion in reagent compartment 804″ in order to impart the desired downward motion of droplets. In FIG. 8C there is a single module 840 that can be moved along the length of the fluid compartment 804. In FIG. 8D there are multiple modules 840 that are controlled in a manner that causes the droplets to move in a determined direction.

FIG. 9A-G show various alternate exemplary reagent compartment 904. Reagent compartment 904 in these figures are similar to reagent compartment 304 as described in FIG. 3B. Reagent layer 910 is a substantially planar and has a first surface (shown as an upper surface) 911 and a second opposing surface (shown as a lower surface) 913 and can be configured such that it contains one or more reagent compartments 904 extending from the first surface wherein each reagent compartment is adapted and configured to form a vessel suitable for housing one or more fluids. These primary reagent storage compartments 904 are further sealable with a frangible seal 906 covering reagent compartment 904 on the second surface 913.

FIG. 9A show a reagent compartment 904 with a lid 947 which covers reagent compartment 904 at an opposing surface from frangible seal 906. Reagent compartment 947 is in communication with a piston 949 internal to reagent chamber 904. The fluid processing apparatus has an actuator 945 that is used to force the fluid out through the frangible seal. In this embodiment, actuator 945 moves downward along axis P1, penetrates through reagent compartment lid 947 and mates with piston 949. Further downward movement of plunger 945 and piston 949 cause the frangible seal 906 to break. Reagent compartment 904 may have a gas 903 in the compartment with the liquid reagent 901.

FIG. 9B shows an alternate pressure source to empty exemplary reagent compartment 904. Heat shrinkable tubing 951 surrounds flexible reagent compartment 904 containing reagent 901. As heat is applied to tubing 951 it constricts, thus causing compartment 904 to collapse and empty the reagent through the reaction layer.

FIGS. 9C-D shows an alternative reagent chamber 904 which can be used to introduce reagents or samples into the reaction area columns or reaction vessels in a reaction layer similar to 320 (see FIG. 3B). This embodiment of reagent chamber 904 contains a needle 909 which is retracted into compartment 904 with its sharp end oriented toward the frangible seal 906. Reagent chamber 904 also contains collapsible side wall bellows 975. Downward pressure along axis P1 from piston 945 presses on the reagent compartment 904 causes the bellows to compress and forces the needle 909 to pierce seal 906. Further compression of the compartment 904 forces the liquid out through the needle as shown in FIG. 9D.

FIGS. 9E-F shows an exemplary embodiment which can be used to introduce reagents or samples into the reaction area columns or reaction vessels in a reaction layer similar to 320 (see FIG. 3B). A needle 909 is part of a plunger inside of compartment 904. When piston 940 compresses the compartment forcing the plunger to move towards the opposite end of the compartment, the needle pierces seal 906 causing liquid to be delivered to a column of vessel in layer 320 (see FIG. 3B).

Reagent compartments 904 can be of different shapes, for example taller or skinnier as shown in FIG. 9G or wider or lower as shown in FIG. 9H. Correspondingly in FIG. 9G the fluid processing apparatus actuator 961 is for example a plunger, piston, or expandable bladder to compress the compartment. In FIG. 9H the fluid processing apparatus actuator 963 is correspondingly, for example, a wedge, cam or roller that is used to move the liquid towards the compartment's exit 906. Other methods of actuation to compress reagent compartments can be used without departing from the scope of the invention.

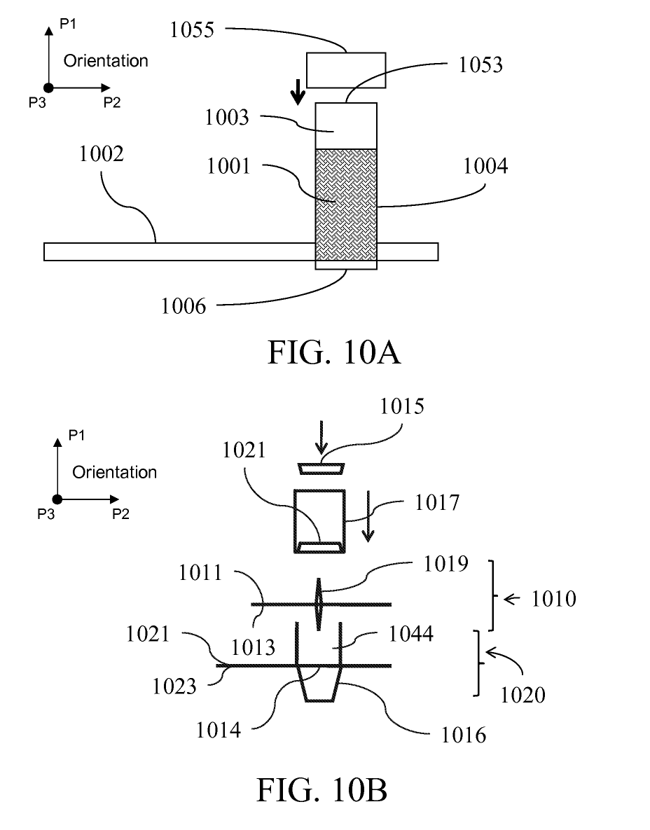

Exemplary reagent compartments 304, 404, 504 and 604, 704, 804 and 904 are usually integrated with the rest of the device. Depending on the application and sample type, it may be desirable to use custom reagents to help develop new fluid handling processes. FIGS. 10A-B show alternate exemplary reagent compartments that enable the use of custom reagents.

FIG. 10A shows an alternate exemplary reagent compartment 1004 with an opening 1053 to accept user added reagent 1001 and a gas 1003. In this embodiment a cap 1055 is used to close off reagent compartment 1004, prior to use. Reagent compartment 1004 may be prefilled or filled by the user and may or may not be permanently attached to substrate 1002 of a reagent layer. These reagent compartments may be shipped, stored or filled separately and attached to substrate 1002 of a reagent layer as needed. This flexibility allows the user to customize the reagents needed for any required processes.

FIG. 10B show a close up side view of reagent layer 1010 and reaction layer 1020 of another fluid handling device 1000. Reagent layer 1010 contains a two headed needle 1019 which passes through and provides a fluid communication path through, for example, a first surface 1011 and, for example, a second surface 1013 of reagent layer 1010. Reaction layer 1020 contains reaction area compartment 1044, reaction area 1014, and reaction area output 1016. Reaction area compartment 1044, arranged on first surface 1021, is open at the top and is in communication with an opposing second surface 1013 of reagent layer 1010. Reaction area output 1016, arranged on second surface 1023, is open at the bottom and is in communication with fluid collection layer 730 (not shown). Together, reaction area compartment 1044, reaction area 1014 and reaction area output 1016 are in fluid communication and comprise a reaction area column. Instead of reagents or samples added or stored, for example, in compartments 304 of reagent layer 310 (see FIG. 3B), reagents or samples can be added or stored in compartment 1017. Examples of reagents that can be stored in compartment 1017 include enzymes or buffers that may aid in the lysis or preprocessing of the samples, solutions that may aid in the stabilization of sample during storage or shipping, or beads that may aid in the disruption of samples. Samples added to compartment 1017 and sealed with lid 1015 can be incubated to allow the enzymes or buffers stored in said compartment to act on the sample. If the reagent in compartment 1017 contain beads, pretreatment can include shaking, vortexing or other mechanical forces such as sonication in order to impart violent motion of the beads. Such violent bead motions are known to those skilled in the arts to disrupt the cell walls of hardy organisms and thereby release analytes into solution. Compartment 1017 has a pierceable septum 1021 on the bottom and a moveable lid 1015 on top. Alternatively, the septum 1021 can be integrated as part of the lid 1015 and the body of compartment 1017 may be at least partially compressible. After the desired pretreatment of the sample, the user pushes compartment 1017 onto a 2 headed needle 1019, such that septum 1021 is pierced by the 2-headed needle 1019. Pressure on lid 1015 or compression on the body of compartment 1017 increases the internal pressure of compartment 1017 and forces the sample or reagents through needle 1019 and into compartment 1044 or other compartments on the reaction layer 1020. Use of the embodiment described in FIG. 10B allows storage or pretreatment of a sample and subsequent processing of the sample through the fluid handling device without needing to open the pretreatment vessel or compartment after the preprocessing steps. Additional manual fluid transfer steps are also not required which may pose contamination risks to the user or to the sample.

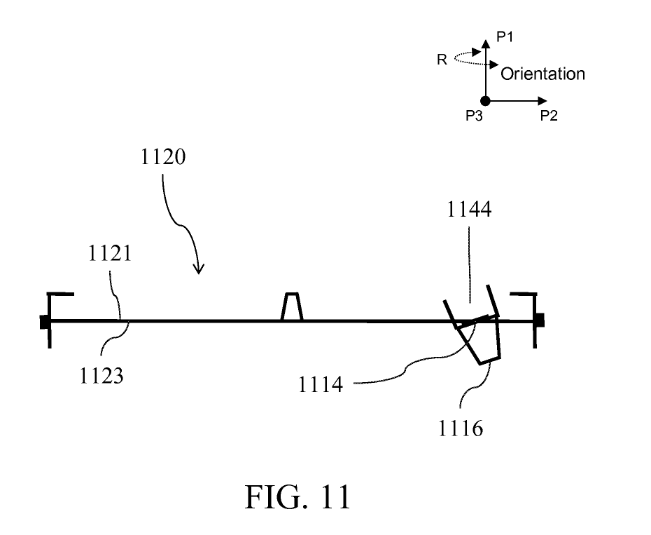

FIG. 11 shows a side view in the plane P2-P3 of an alternate reaction layer 1120. Reaction layer 1120 has a circular cross section in the plane P2-P3. Reaction layer 1120 contains reaction area compartment 1144, reaction area 1114, and reaction area output 1116. Reaction area compartment 1144, arranged on first surface 1121, is open at the top and is in communication with a second surface of a reagent layer such as 310 and 510 allowing it to receive fluids from said reagent layer. Reaction area output 1116, arranged on second surface 1123, is open at the bottom and is in communication with a fluid collection layer (not shown). Together, reaction area compartment 1044, reaction area 1014 and reaction area output 1016 are in fluid communication and comprise a reaction area column. This column is positionable near the outer edge of reaction layer 1120 in the plane P2-P3 and is at an angle relative to layer 1120. Alternatively the reaction area column is mountable on swivels analogous to a swinging bucket in a swinging bucket centrifuge. Rotation R of layer 1120 about axis P1, either independently or in concert with the other layers, can impart a centrifugal force on the reaction area column which can drive fluids contained in compartment 1144, through reaction area 1114 and through outlet 1116.

FIG. 12A shows an fluid processing apparatus 1250 that manipulates the fluid processing device 300 in manual mode without the requirement of an electrical power source. Another embodiment of the fluid processing apparatus 1260, shown in FIG. 12B, automates all the positional motion of the device and emptying of reagent/gas compartments through the reaction area. Unique device identification 1272 is read by the fluid processing apparatus 1260 to ensure running the correct protocol on the device. Suitable unique identifiers 1272 include those discussed above in FIG. 3. The fluid processing apparatus 1260 may have a communication interface to an external controller 1270 that is capable of controlling the fluid processing apparatus 1260 by sending commands over the communication media 1274.

Fluid processing apparatus 1260 may manipulate multiple fluid processing devices 300 at one time. FIG. 12C show a fluid processing apparatus that has multiple slots, one for each fluid processing device 300. These slots may be simple, for example a holder for the fluid processing device where each device 300 is movable to a fluid device manipulation portion of the fluid processing apparatus or each slot may be more independent with separate fluid device manipulation capability in each slot.

In the exemplary embodiments shown in FIGS. 2-12 alignment of the various components is accomplishable by linear, rotary or combined motion. Features described in these embodiments may be utilized individually or in any combination.

### III. Fluid Handling Devices and Communication Network

FIG. 12D shows a modular and scalable system comprised of a controller 1270 and more than one fluid processing apparatus 1260. Controller 1270 communicates with each fluid processing apparatus 1260 over communication media 1274. Communication media 1274 may be a wired point-to-point or multi-drop configuration. Examples of wired communication media 1274 include Ethernet, USB, and RS-232. Alternatively communication media 1274 may be wireless including radio frequency (RF) and optical. The fluid processing apparatus 1260 may have one or more slots for fluid processing devices 300.

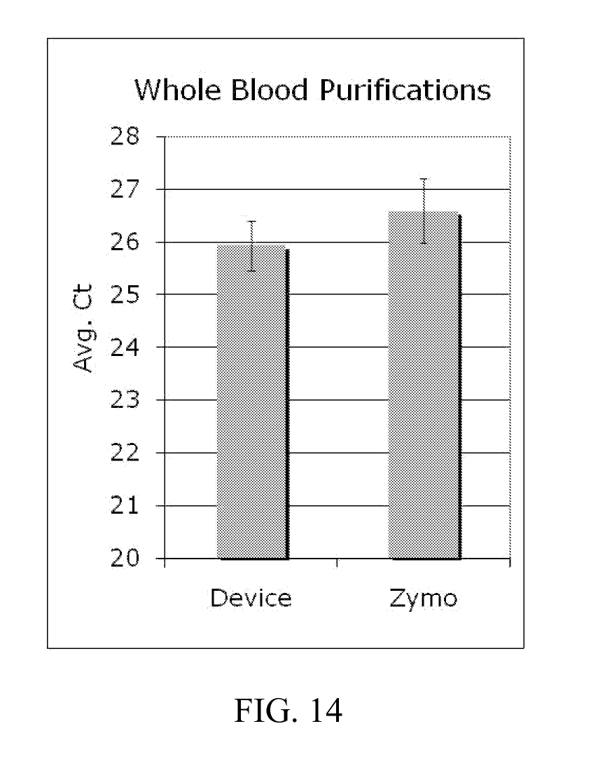

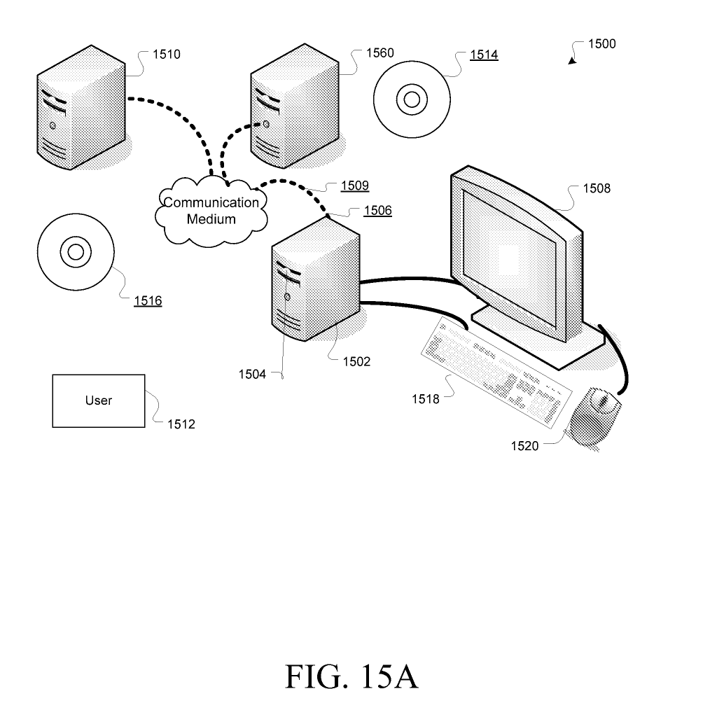

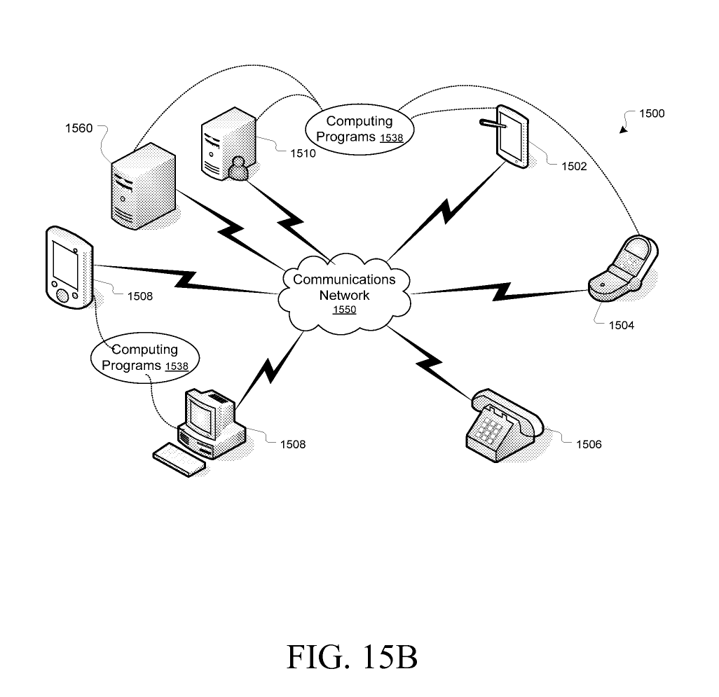

To further appreciate the networked configurations of multiple fluid handling devices and diagnostic devices in a communication network, FIG. 15A is a block diagram showing a representative example logic device through which a browser can be accessed to control and/or communication with fluid handling devices and/or diagnostic devices described above. A computer system (or digital device) 1500, which may be understood as a logic apparatus adapted and configured to read instructions from media 1514 and/or network port 1506, is connectable to a server 1510, and has a fixed media 1516. The computer system 1500 can also be connected to the Internet or an intranet. The system includes central processing unit (CPU) 1502, disk drives 1504, optional input devices, illustrated as keyboard 1518 and/or mouse 1520 and optional monitor 1508. Data communication can be achieved through, for example, communication medium 1509 to a server 1510 at a local or a remote location. The communication medium 1509 can include any suitable means of transmitting and/or receiving data. For example, the communication medium can be a network connection, a wireless connection, or an internet connection. It is envisioned that data relating to the use, operation or function of the fluid handling devices and/or diagnostic devices (shown together for purposes of illustration here as 1560) can be transmitted over such networks or connections. The computer system can be adapted to communicate with a user (users include healthcare providers, physicians, lab technicians, nurses, nurse practitioners, patients, and any other person or entity which would have access to information generated by the system) and/or a device used by a user. The computer system is adaptable to communicate with other computers over the Internet, or with computers via a server. Moreover the system is configurable to activate one or more devices associated with the network (e.g., diagnostic devices and/or fluid handling devices) and to communicate status and/or results of tests performed by the diagnostic devices and/or diagnostic systems.

As is well understood by those skilled in the art, the Internet is a worldwide network of computer networks. Today, the Internet is a public and self-sustaining network that is available to many millions of users. The Internet uses a set of communication protocols called TCP/IP (i.e., Transmission Control Protocol/Internet Protocol) to connect hosts. The Internet has a communications infrastructure known as the Internet backbone. Access to the Internet backbone is largely controlled by Internet Service Providers (ISPs) that resell access to corporations and individuals.

The Internet Protocol (IP) enables data to be sent from one device (e.g., a phone, a Personal Digital Assistant (PDA), a computer, etc.) to another device on a network. There are a variety of versions of IP today, including, e.g., IPv4, IPv6, etc. Other IPs are no doubt available and will continue to become available in the future, any of which can, in a communication network adapted and configured to employ or communicate with one or more fluid handling devices and/or diagnostic devices, be used without departing from the scope of the invention. Each host device on the network has at least one IP address that is its own unique identifier and acts as a connectionless protocol. The connection between end points during a communication is not continuous. When a user sends or receives data or messages, the data or messages are divided into components known as packets. Every packet is treated as an independent unit of data and routed to its final destination—but not necessarily via the same path.

The Open System Interconnection (OSI) model was established to standardize transmission between points over the Internet or other networks. The OSI model separates the communications processes between two points in a network into seven stacked layers, with each layer adding its own set of functions. Each device handles a message so that there is a downward flow through each layer at a sending end point and an upward flow through the layers at a receiving end point. The programming and/or hardware that provides the seven layers of function is typically a combination of device operating systems, application software, TCP/IP and/or other transport and network protocols, and other software and hardware.

Typically, the top four layers are used when a message passes from or to a user and the bottom three layers are used when a message passes through a device (e.g., an IP host device). An IP host is any device on the network that is capable of transmitting and receiving IP packets, such as a server, a router or a workstation. Messages destined for some other host are not passed up to the upper layers but are forwarded to the other host. The layers of the OSI model are listed below. Layer 7 (i.e., the application layer) is a layer at which, e.g., communication partners are identified, quality of service is identified, user authentication and privacy are considered, constraints on data syntax are identified, etc. Layer 6 (i.e., the presentation layer) is a layer that, e.g., converts incoming and outgoing data from one presentation format to another, etc. Layer 5 (i.e., the session layer) is a layer that, e.g., sets up, coordinates, and terminates conversations, exchanges and dialogs between the applications, etc. Layer-4 (i.e., the transport layer) is a layer that, e.g., manages end-to-end control and error-checking, etc. Layer-3 (i.e., the network layer) is a layer that, e.g., handles routing and forwarding, etc. Layer-2 (i.e., the data-link layer) is a layer that, e.g., provides synchronization for the physical level, does bit-stuffing and furnishes transmission protocol knowledge and management, etc. The Institute of Electrical and Electronics Engineers (IEEE) sub-divides the data-link layer into two further sub-layers, the MAC (Media Access Control) layer that controls the data transfer to and from the physical layer and the LLC (Logical Link Control) layer that interfaces with the network layer and interprets commands and performs error recovery. Layer 1 (i.e., the physical layer) is a layer that, e.g., conveys the bit stream through the network at the physical level. The IEEE sub-divides the physical layer into the PLCP (Physical Layer Convergence Procedure) sub-layer and the PMD (Physical Medium Dependent) sub-layer.

Wireless networks can incorporate a variety of types of mobile devices, such as, e.g., cellular and wireless telephones, PCs (personal computers), laptop computers, wearable computers, cordless phones, pagers, headsets, printers, PDAs, etc. and suitable for use in a system or communication network that includes one or more diagnostic devices and/or one or more fluid handling devices. For example, mobile devices may include digital systems to secure fast wireless transmissions of voice and/or data. Typical mobile devices include some or all of the following components: a transceiver (for example a transmitter and a receiver, including a single chip transceiver with an integrated transmitter, receiver and, if desired, other functions); an antenna; a processor; display; one or more audio transducers (for example, a speaker or a microphone as in devices for audio communications); electromagnetic data storage (such as ROM, RAM, digital data storage, etc., such as in devices where data processing is provided); memory; flash memory; and/or a full chip set or integrated circuit; interfaces (such as universal serial bus (USB), coder-decoder (CODEC), universal asynchronous receiver-transmitter (UART), phase-change memory (PCM), etc.). Other components can be provided without departing from the scope of the invention.

Wireless LANs (WLANs) in which a mobile user can connect to a local area network (LAN) through a wireless connection may be employed for wireless communications between one or more diagnostic devices and/or fluid handling devices. Wireless communications can include communications that propagate via electromagnetic waves, such as light, infrared, radio, and microwave. There are a variety of WLAN standards that currently exist, such as Bluetooth®, IEEE 802.11, and the obsolete HomeRF.

By way of example, Bluetooth products may be used to provide links between mobile computers, mobile phones, portable handheld devices, personal digital assistants (PDAs), and other mobile devices and connectivity to the Internet. Bluetooth is a computing and telecommunications industry specification that details how mobile devices can easily interconnect with each other and with non-mobile devices using a short-range wireless connection. Bluetooth creates a digital wireless protocol to address end-user problems arising from the proliferation of various mobile devices that need to keep data synchronized and consistent from one device to another, thereby allowing equipment from different vendors to work seamlessly together.

An IEEE standard, IEEE 802.11, specifies technologies for wireless LANs and devices. Using 802.11, wireless networking may be accomplished with each single base station supporting several devices. In some examples, devices may come pre-equipped with wireless hardware or a user may install a separate piece of hardware, such as a card, that may include an antenna. By way of example, devices used in 802.11 typically include three notable elements, whether or not the device is an access point (AP), a mobile station (STA), a bridge, a personal computing memory card International Association (PCMCIA) card (or PC card) or another device: a radio transceiver; an antenna; and a MAC (Media Access Control) layer that controls packet flow between points in a network.

In addition, Multiple Interface Devices (MIDs) may be utilized in some wireless networks. MIDs may contain two independent network interfaces, such as a Bluetooth interface and an 802.11 interface, thus allowing the MID to participate on two separate networks as well as to interface with Bluetooth devices. The MID may have an IP address and a common IP (network) name associated with the IP address.

Wireless network devices may include, but are not limited to Bluetooth devices, WiMAX (Worldwide Interoperability for Microwave Access), Multiple Interface Devices (MIDs), 802.11x devices (IEEE 802.11 devices including, 802.11a, 802.11b and 802.11g devices), HomeRF (Home Radio Frequency) devices, Wi-Fi (Wireless Fidelity) devices, GPRS (General Packet Radio Service) devices, 3 G cellular devices, 2.5 G cellular devices, GSM (Global System for Mobile Communications) devices, EDGE (Enhanced Data for GSM Evolution) devices, TDMA type (Time Division Multiple Access) devices, or CDMA type (Code Division Multiple Access) devices, including CDMA2000. Each network device may contain addresses of varying types including but not limited to an IP address, a Bluetooth Device Address, a Bluetooth Common Name, a Bluetooth IP address, a Bluetooth IP Common Name, an 802.11 IP Address, an 802.11 IP common Name, or an IEEE MAC address.

Wireless networks can also involve methods and protocols found in, Mobile IP (Internet Protocol) systems, in PCS systems, and in other mobile network systems. With respect to Mobile IP, this involves a standard communications protocol created by the Internet Engineering Task Force (IETF). With Mobile IP, mobile device users can move across networks while maintaining their IP Address assigned once. See Request for Comments (RFC) 3344. NB: RFCs are formal documents of the Internet Engineering Task Force (IETF). Mobile IP enhances Internet Protocol (IP) and adds a mechanism to forward Internet traffic to mobile devices when connecting outside their home network. Mobile IP assigns each mobile node a home address on its home network and a care-of-address (CoA) that identifies the current location of the device within a network and its subnets. When a device is moved to a different network, it receives a new care-of address. A mobility agent on the home network can associate each home address with its care-of address. The mobile node can send the home agent a binding update each time it changes its care-of address using Internet Control Message Protocol (ICMP).

In basic IP routing (e.g., outside mobile IP), routing mechanisms rely on the assumptions that each network node always has a constant attachment point to the Internet and that each node's IP address identifies the network link it is attached to. Nodes include a connection point, which can include a redistribution point or an end point for data transmissions, and which can recognize, process and/or forward communications to other nodes. For example, Internet routers can look at an IP address prefix or the like identifying a device's network. Then, at a network level, routers can look at a set of bits identifying a particular subnet. Then, at a subnet level, routers can look at a set of bits identifying a particular device. With typical mobile IP communications, if a user disconnects a mobile device from the Internet and tries to reconnect it at a new subnet, then the device has to be reconfigured with a new IP address, a proper netmask and a default router. Otherwise, routing protocols would not be able to deliver the packets properly.

Computing system 1500, described above, can be deployed as part of a computer network that includes one or more diagnostic devices and/or fluid handling devices. In general, the above description for computing environments applies to both server computers and client computers deployed in a network environment. FIG. 15B illustrates an exemplary illustrative networked computing environment 1500, with a server in communication with client computers via a communications network 1550. As shown in FIG. 15B, server 1510 may be interconnected via a communications network 1550 (which may be either of, or a combination of a fixed-wire or wireless LAN, WAN, intranet, extranet, peer-to-peer network, virtual private network, the Internet, or other communications network) with a number of client computing environments such as tablet personal computer 1502, mobile telephone 1504, telephone 1506, personal computer 1502, and personal digital assistant 1508. In a network environment in which the communications network 1550 is the Internet, for example, server 1510 can be dedicated computing environment servers operable to process and communicate data to and from client computing environments via any of a number of known protocols, such as, hypertext transfer protocol (HTTP), file transfer protocol (FTP), simple object access protocol (SOAP), or wireless application protocol (WAP). Other wireless protocols can be used without departing from the scope of the invention, including, for example Wireless Markup Language (WML), DoCoMo i-mode (used, for example, in Japan) and XHTML Basic. Additionally, networked computing environment 1500 can utilize various data security protocols such as secured socket layer (SSL) or pretty good privacy (PGP). Each client computing environment can be equipped with operating system 1538 operable to support one or more computing applications, such as a web browser (not shown), or other graphical user interface (not shown), or a mobile desktop environment (not shown) to gain access to server computing environment 1500.

In operation, a user (not shown) may interact with a computing application running on a client computing environment to obtain desired data and/or computing applications. The data and/or computing applications may be stored on server computing environment 1500 and communicated to cooperating users through client computing environments over exemplary communications network 1550. A participating user may request access to specific data and applications housed in whole or in part on server computing environment 1500. These data may be communicated between client computing environments and server computing environments for processing and storage. Server computing environment 1500 may host computing applications, processes and applets for the generation, authentication, encryption, and communication data and applications and may cooperate with other server computing environments (not shown), third party service providers (not shown), network attached storage (NAS) and storage area networks (SAN) to realize application/data transactions.

### IV. Methods of Fluid Handling

### EXAMPLE 1

Methods according to this disclosure include purification and concentration of DNA from a sample through a silica filter. Commercial kits, such as the Qiagen DNeasy®Blood and Tissue Kits (Qiagen 69504) are commonly used for this purpose. A pilot experiment was performed in order to test whether compression of a collapsible reagent compartment and application of an external pressure source is sufficient to perform such a purification.

For a control Qiagen purification, 200 uL of ˜2.5 ng/uL human genomic DNA sample was mixed with 200 uL of Buffer AL and 200 uL EtOH. This mixture was applied to the silica column and processed by centrifugation per manufacturer's instruction. Each sample was washed with 500 uL each of buffer AW1 and AW2 followed by elution with 100 uL Buffer AE.

The same reagents were used to test the invention, except that the process was modified in the following manner. The sample mixtures, and the washes were passed through the column, not by centrifugation, but by pressure fit sealing of a standard laboratory transfer pipette to the silica column and compressing the pipette bulb. The last wash was dried by application of ˜15 psi air through the column for 3 minutes. The elution buffer was also passed through the column with 15 psi air for approximately 15 seconds. Four control and prototype purifications were performed each. The amount of DNA in each eluate was quantitated by quantitative polymerase chain reaction (PCR) using primers designed for the rnaseP gene. The data is shown in FIG. 13. The average threshold cycle (Ct's) for the prototype and the Qiagen control samples are nearly identical, suggesting that use of inexpensive compressible reagent compartments and external pressure source can be used to drive fluids through the column to perform DNA purification.

### EXAMPLE 2

A ZR Genomic DNA II Kit™ from Zymo Research (Orange, Calif.) can be used to purify DNA from human blood. Purified DNA from blood can be used in downstream analytical process to provide valuable information such as determination of the genetic relationships between individuals, assessment of a patient's likely response to a therapeutic, and the identification of possible infectious agents.

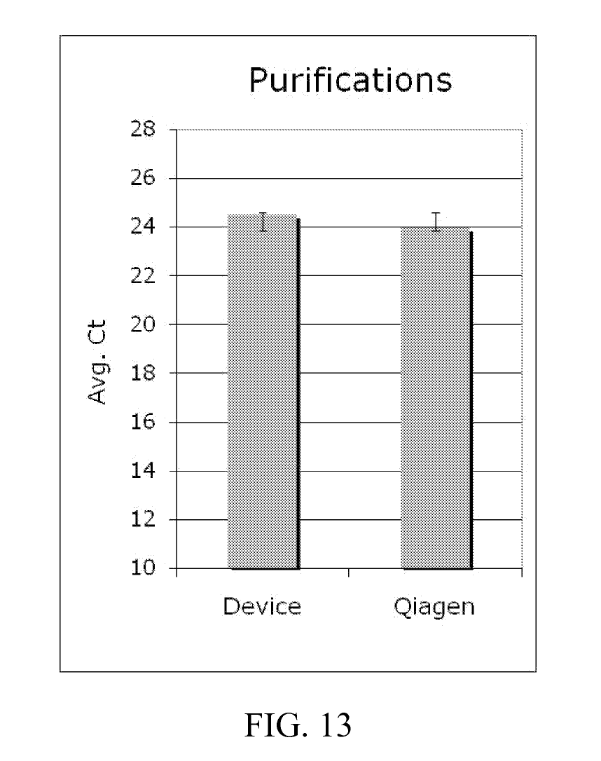

To test the invention, a device similar to device 500 shown in FIG. 5 was used. Four control purifications of DNA from 50 uL of human blood per purification were performed according to the manufacturer's protocol. The same Zymo ZR Genomic DNA II Kit™ reagents as used in the control purifications were aspirated into common laboratory disposable plastic Pasteur pipettes for use as the reagent compartments and attached to layer 510. The reaction vessel and eluate collection vessels were common laboratory test tubes attached to layers 520 and 530 respectively. The reaction area columns used in the device were the same spin columns supplied with the ZR Genomic DNA II Kit™ and attached to layer 520. Four purifications of 50 uL human blood were performed on the device using the manufacturer's recommended protocol, but with the following modifications. The sample mixtures, and the washes were passed through the column, not by centrifugation, but by sealing an external pressure source to the reaction area column and passing 15 psi (pounds per square inch) for 15-30 seconds. The last wash was dried by application of ˜15 psi air through the column for 3 minutes. The elution buffer was also passed through the column with 15 psi air for approximately 5 seconds. The amount of DNA in each eluate was quantitated by quantitative PCR using primers designed for the rnaseP gene. The data is shown in FIG. 14. The average threshold cycle (Ct's) for the prototype and the Zymo control samples are nearly identical, indicating that use of inexpensive compressible reagent compartments and an external pressure source can be used to purify DNA from whole blood with an embodiment of the invention.

### EXAMPLE 3

Evaluation of commercially available rapid influenza diagnostic tests (RIDTs) by the CDC (Evaluation of Rapid Influenza Diagnostic Testes for Detection of Novel Influenza A (H1N1) Virus. Morbidity and Mortality Weekly Report. 58(30): 826-829. Aug. 7, 2009) revealed shortcomings with the ability of these tests to detect the H1N1 strain of influenza. These tests miss many cases of the H1N1 virus, with a detection rate of only 40-69% and none of these tests can distinguish between the different strains. In this study, the CDC used purification of nucleic acids from the samples followed by rRT-PCR assays (5′ fluorogenic exonuclease assay) as the “gold standard” to detect the presence of influenza. The rRT-PCR tests used by the CDC are well over 99% sensitive and can distinguish between the various influenza strains. While these types of rRT-PCR tests are available to the general public, they typically require that the sample be sent to a central laboratory for testing. The requirement for well trained personnel, at well equipped laboratories, at locations remote from the patient to perform these tests increases the cost of these tests. Transport of the sample to these laboratories can lead to sample degradation and places a requirement for sample stabilization. In addition, the delays (days or weeks) in reporting the results back to the doctor and patient reduces the utility of the information. Using the inventions described above, these highly sensitive tests can be performed at the doctor's office safely, and in a time and cost effective manner. By way of example, a sample from the patient (for example nasal swabs) is collected and placeable into a reaction compartment 552 of device 500 as described in FIG. 5. The user places device 500 into a Sample to Answer fluid processing apparatus (see 260 in FIG. 2). The fluid processing apparatus automatically carries out the process as described in FIG. 14 and FIGS. 5F-S. The influenza virus is a relatively easy to lyse target and the target nucleic acid is released into the solution almost immediately upon addition of the lysis buffer (FIGS. 5H-I). Addition of binding buffer (FIG. 5K), binding of the lysate to the reaction area (FIGS. 5L-O), washing of the reaction area (FIGS. 5P-Q), and elution of the purified nucleic acids into collection compartment 524 (FIGS. 5R-S) are all carried out automatically by the fluid processing apparatus. Collection compartment 524 contain lyophilized reagents (enzymes, buffers, primers and probes) needed for the rRT-PCR detection reaction. Addition of the purified eluate to this compartment, reconstitutes the reaction and the fluid processing apparatus executes thermal cycling required for this reaction, collects the fluoresence signal, and reports the results. The user collects the results and disposes of the device 500 in the proper manner.

### EXAMPLE 4

Rapid identification of potential bacterial infections is a growing need. For example, Clostridium difficile infection (CDI) has been cited by the CDC as an emerging threat. C. difficile is a spore forming bacillus bacteria that can infect the elderly and patients with weakened immune systems. Patients treated with antibiotics for other infections are also at elevated risk for CDI. Infection with C. difficile can result in Colitis, other intestinal conditions, sepsis and death. Both the rates of CDI and the severity of symptoms from these infections have been rising in recent years and it is believed that the emergence of more virulent strains is, in part, to blame. Since the majority of human cases of CDI occur in association with inpatient stays in hospitals or long-term care facilities, technologies to rapidly detect CDI and to identify the underlying strains is of great interest. MDx is ideally suited for this role.

C. difficile is an example of a target with hardy cell walls that must be breached to release the nucleic acids for purification and detection. By way of example, different configuration of the invention can be utilized in the analysis of C. difficile containing samples. As a first example, samples suspected of containing C. difficile can be added to a septum containing reagent compartment with lysis buffer and glass beads similar to that described in FIG. 10B. This compartment is shaken or vortexed with the sample to disrupt the cell walls and release the cellular content into the buffer. The user then attaches this compartment to the device via the 2 headed needle on the reagent layer. The sample is then processed in a manner similar to that described in FIG. 10B and FIG. 5.

As a second example, a fluid handling device can be used that has an integrated reaction vessel in the reaction layer, a second detachable reaction vessel in the reaction layer and a detachable elution vessel in the fluid collection layer. The sample can be added to the integrated reaction vessel. The user selects a detachable reaction vessel that contains reagents (for example lyophilized enzymes) optimized for degradation of the C. difficile cell wall and attaches it to the reaction layer. The user also selects a detachable elution vessel that contains reagents (for example buffers, enzymes, primers and probes) optimized for detection of C. difficile and attaches it to the fluid collection layer. Both the detachable reaction and elution vessel may be sealed with a temporary Aluminum lid seal for storage and transport. The option of selecting and attaching detachable reaction and elution vessels allows the user to select reagents optimized for their sample target. These detachable reaction and elution vessels may be supplied as part of a kit with all necessary components or may be ordered separately as needed. Once the user had attached the required reaction and and elution vessel to the device and added the sample to the integrated reaction vessel on the device, the user places the device into the fluid processing apparatus and processes the sample as described previously.

### V. Kits for Fluid Handling

Kits are also contemplated as an aspect of the invention. Suitable kits for extracting nucleic acid from a sample, include, for example, a device with prefilled reagent compartments packaged in an hermitically sealed pouch. The user opens the pouch and inserts the device into an fluid processing apparatus capable of running the fluid handling protocol. Kits may be differentiated by one or more of the following:
by the specific number of reagent compartments, reaction vessels, reaction area columns, eluate collection vessels, and waste compartments.
by the specific shape of reagent compartments, reaction vessels, reaction area columns, eluate collection vessels, and waste compartments.
by the specific reagents and volume of reagents contained in the reagent compartments reaction vessels, reaction area columns, eluate collection vessels, and waste compartments.
by whether they contain custom reagents in the reagent compartments, reaction vessels, reaction area columns, eluate collection vessels, and waste compartments.
by the methods required to operate the device (for example rotary or linear form factor)
by whether the reagent compartments, reaction vessels, reaction area columns, eluate collection vessels, and waste compartments are integrated as part of the device or attachable to the device by the user.
by whether the reagent, reaction area, and fluid collection layers are separated in the kit or integrated into a single fluid handling device.
by whether the reagent, reaction area, and fluid collection layers are inserted separately into the fluid processing apparatus or as an assembled fluid handling device.
   Customers may order kits with their own specified reagents or even customer provided reagents in the reagent compartments. The devices in the kits are matched to the fluid processing apparatus that processes them. In this way there may be a variety of form factors for kits in rotary or linear configurations. Kits may also be provided that contain a device with an output port to transfer the elution fluid immediately to a detection device. Kits may also be provided such that the eluate collection vessel is also used for detection. Other differentiation of kits can be used without departing from the scope of the invention. Kits may also be provided with an adapter designed to enable the fluid handling device to mate with a specific fluid processing system.

### VI. Methods of Manufacturing

Devices are manufacturable using one or more of vacuum, pressure, thermal forming, blow molding, and injection molding processes. Resins used in these processes will depend for example, on the reagents being packaged and on the size of the manufactured device. Reagent compartments that contain solid or liquid reagents are manufacturable with blow, fill, seal and modified blow, fill, seal manufacturing techniques. Reagent compartments may be manufactured individually or in ganged configurations. Existing off-the-shelf components for reagent compartments, reaction vessels, eluate collection vessels may be integrated into the final device using custom holders for the parts. Alternatively the device design may be entirely custom with no off-the-shelf parts. The device may consist of three layers with independent motion or one or more layers may be integrated together and may be desirable to produce lower cost devices. Devices may be manufactured that combine a fluid handling device with detection device. Other methods of manufacturing can be used without departing from the scope of the invention.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A fluid handling device comprising:
an inlet for receiving a sample;
a reagent layer comprising,
a substrate having a first surface and an opposing second surface,
at least one reagent storage compartment configured to hold a reagent, and
a seal in communication with the at least one reagent storage compartment; and
a reaction layer having a first surface and an opposing second surface comprising,
a reaction area, and
an outlet in communication with the reaction area,
wherein the reagent layer and reaction layer are adapted and configured to permit movement of at least one of the reagent layer and the reaction layer in a plane relative to the other layer.

2. The fluid handling device of claim 1 further comprising a reagent layer support extending from the substrate.

3. The fluid handling device of claim 1 further comprising a reaction layer support.

4. The fluid handling device of claim 1 further comprising a shoulder.

5. The fluid handling device of claim 1 wherein the seal is selectively openable.

6. The fluid handling device of claim 1 wherein at least one of the at least one reagent storage compartment is compressible.

7. The fluid handling device of claim 1 wherein the reagent layer further comprises two or more reagent storage compartments and further wherein at least two of the reagent storage compartments of the reagent layer are in fluid communication.

8. The fluid handling device of claim 7 wherein the two or more reagent storage compartments contain two or more reagents.

9. The fluid handling device of claim 1 wherein the reagent storage compartment further comprises a tip.

10. The fluid handling device of claim 9 wherein the tip is configured to enable at least one of mixing of material within the reaction area, transfer of material from a first reaction area to a second reaction area, and transfer of material from a reaction area to a fluid collection compartment.

11. The fluid handling device of claim 9 wherein the tip further comprises a selectively openable seal.

12. The fluid handling device of claim 1 wherein the reaction area further comprises one or more filters.

13. The fluid handling device of claim 1 wherein the reaction area further comprises a reaction vessel.

14. The fluid handling device of claim 13 wherein the reaction vessel further comprises a reagent.

15. The fluid handling device of claim 1 further comprising a fluid collection compartment.

16. The fluid handling device of claim 1 further comprising an external positioning feature adapted and configured to engage the reagent layer with the reaction layer.

17. The fluid handling device of claim 1 further comprising a fluid collection layer.

18. The fluid handling device of claim 17 wherein the fluid collection layer further comprises one or more inlets.

19. The fluid handling device of claim 17 further comprising one or more materials adapted and configured to absorb waste.

20. The fluid handling device of claim 17 wherein the fluid collection layer further comprises one or more fluid collection compartments.

21. The fluid handling device of claim 20 wherein at least one of the one or more fluid collection compartments contains a reagent.

22. The fluid handling device of claim 20 wherein the fluid collection layer is sealable.

23. The fluid handling device of claim 1 wherein at least one of the reagent layer and the reaction layer is nestable within the other layer.

24. The fluid handling device of claim 1 wherein the reagent compartment of the reagent layer is adaptable to form a pressure tight seal.

25. The fluid handling device of claim 1 wherein the reagent layer and reaction layer are adapted and configured to move at least one of about an axis and along and axis.

26. The fluid handling device of claim 17 wherein at least one of the reagent layer and the reaction layer are adapted and configured to permit movement in a plane relative to the fluid collection layer.

27. The fluid handling device of claim 1 wherein the reagent layer and reaction layer are integrally formed.

28. The fluid handling device of claim 17 further comprising an external positioning feature adapted and configured to engage the reaction layer with the fluid collection layer.

29. The fluid handling device of claim 1 wherein the external positioning features are adapted and configured to prevent movement of at least one of the reagent layer and the reaction layer relative to the other layer.

30. The fluid handling device of claim 17 wherein the external positioning features are adapted and configured to at least one of prevent movement of at least one of the reagent layer, the reaction layer and the fluid collection layer relative to at least one other layer and permit movement of at least one of the reagent layer, the reaction layer and the fluid collection layer relative to at least one other layer.

31. The fluid handling device of claim 1 wherein the reaction layer is in fluid communication with a first reagent storage compartment at a first time and a second reagent storage compartment at a second time.

32. The fluid handling device of claims 1 and 17 further comprising one or more device identification components.

33. The fluid handling device of claim 32 wherein the one or more device identification components are adapted and configured to identify one or more of each of serial number, manufacturer, lot number, date codes, reagent type, reagent volume, reaction area type, process identification, process parameters needed to run the process, and calibration parameter.

34. The fluid handling device of claim 33 wherein the one or more device identification components are associated with at least one or more of the fluid handling device, the reagent layer, the reaction layer, and the fluid collection compartment.

35. The fluid handling device of claim 34 wherein the one or more device identification components associated with at least one or more of the fluid handling device, the reagent layer, the reaction layer, and the fluid collection compartment are adapted and configured to communicate information between the one or more of the fluid handling device, the reagent layer, the reaction layer, and the fluid collection compartment.

36. A method of processing a sample for diagnostic testing comprising:
obtaining a sample;
inserting a sample into a reagent layer further comprising, a substrate having a first surface and an opposing second surface, one or more reagent storage compartments configured to hold a reagent, and one or more seals enclosing the one or more reagent storage compartments,
reacting the sample in a reaction layer having a first surface and an opposing second surface comprising, a reaction area, and an outlet in communication with the reaction area, wherein the reagent layer and reaction layer are adapted and configured to permit movement of at least one of the reagent layer and the reaction layer in a plane relative to the other layer; and
processing the sample without human interaction with the sample after the step of inserting the sample into the reagent layer.

37. The method of claim 36 further comprising the step of delivering at least one processed sample to the diagnostic machine.

38. The method of claim 37 further comprising the step of analyzing the at least one processed sample.

39. The method of claim 36 wherein the step of processing the sample includes one or more of adding a lysis buffer to the sample, adding a binding buffer to the sample, binding the sample to a reaction area, emptying a fluid into a waste container; adding a wash buffer; adding an elution buffer; and eluting the sample.

40. The method of claim 36 further comprising the step of controlling at least one of a temperature, a reaction time, and a motion.

41. The method of claim 36 further comprising the step of analyzing the processed sample.

42. The method of claim 36 wherein the sample is a biological sample.

43. The method of claim 42 wherein the biological sample is selected from the group comprising blood, nasal washes, suspensions of particulates, dirt, feces, cellular suspensions, buccal swabs, nucleic acids, protein suspensions, and mixtures of compounds.

44. The method of claim 36 wherein the diagnostic device is selected from the group comprising molecular diagnostic devices, polymerase chain reaction devices, isothermal amplification devices, lateral flow devices, devices employing arrays, electrochemical detection devices, optical detection devices, nucleic acid sequencers.

45. The method of claim 36 further comprising the step of delivering at least two processed samples to the diagnostic machine.

46. A system adapted and configured to process fluid, the fluid processing system comprising:
a diagnostic device; and
a fluid handling device comprising,
an inlet for receiving a sample,
a reagent layer comprising,
a substrate having a first surface and an opposing second surface,
at least one reagent storage compartment configured to hold a reagent,
a seal in communication with the at least one reagent storage compartment,
a reaction layer having a first surface and an opposing second surface comprising,
a reaction area, and
an outlet in communication with the reaction area,
wherein the reagent layer and reaction layer are adapted and configured to permit movement of at least one of the reagent layer and the reaction layer in a plane relative to the other layer.

47. The fluid processing system of claim 46 further comprising a reagent layer support extending from the substrate.

48. The fluid processing system of claim 46 further comprising a reaction layer support.

49. The fluid processing system of claim 46 further comprising a shoulder.

50. The fluid processing system of claim 46 wherein the seal is selectively openable.

51. The fluid processing system of claim 46 wherein at least one of the at least one reagent storage compartment is compressible.

52. The fluid processing system of claim 46 wherein the reagent layer further comprises two or more reagent storage compartments and further wherein at least two of the reagent storage compartments of the reagent layer are in fluid communication.

53. The fluid processing system of claim 52 wherein the two or more reagent storage compartments contain two or more reagents.

54. The fluid processing system of claim 46 wherein the reagent storage compartment further comprises a tip.

55. The fluid processing system of claim 54 wherein the tip is configured to enable at least one of mixing of material within the reaction area, transfer of material from a first reaction area to a second reaction area, and transfer of material from a reaction area to a fluid collection compartment.

56. The fluid processing system of claim 54 wherein the tip further comprises a selectively openable seal.

57. The fluid processing system of claim 46 wherein the reaction area further comprises one or more filters.

58. The fluid processing system of claim 46 wherein the reaction area further comprises a reaction vessel.

59. The fluid processing system of claim 58 wherein the reaction vessel further comprises a reagent.

60. The fluid processing system of claim 46 further comprising a fluid collection compartment.

61. The fluid processing system of claim 46 further comprising an external positioning feature adapted and configured to engage the reagent layer with the reaction layer.

62. The fluid processing system of claim 46 further comprising a fluid collection layer.

63. The fluid processing system of claim 62 wherein the fluid collection layer further comprises one or more inlets.

64. The fluid processing system of claim 62 further comprising one or more materials adapted and configured to absorb waste.

65. The fluid processing system of claim 62 wherein the fluid collection layer further comprises one or more fluid collection compartments.

66. The fluid processing system of claim 65 wherein at least one of the one or more fluid collection compartments contains a reagent.

67. The fluid processing system of claim 65 wherein the fluid collection layer is sealable.

68. The fluid processing system of claim 46 wherein at least one of the reagent layer and the reaction layer is nestable within the other layer.

69. The fluid processing system of claim 46 wherein the reagent compartment of the reagent layer is adaptable to form a pressure tight seal.

70. The fluid processing system of claim 46 wherein the reagent layer and reaction layer are adapted and configured to move at least one of about an axis and along and axis.

71. The fluid processing system of claim 62 wherein at least one of the reagent layer and the reaction layer are adapted and configured to permit movement in a plane relative to the fluid collection layer.

72. The fluid processing system of claim 46 wherein the reagent layer and reaction layer are integrally formed.

73. The fluid processing system of claim 62 further comprising an external positioning feature adapted and configured to engage the reaction layer with the fluid collection layer.

74. The fluid processing system of claim 46 wherein the external positioning features are adapted and configured to prevent movement of at least one of the reagent layer and the reaction layer relative to the other layer.

75. The fluid processing system of claim 62 wherein the external positioning features are adapted and configured to prevent movement of at least one of the reagent layer, the reaction layer and the fluid collection layer relative to at least one other layer.

76. The fluid processing system of claim 46 wherein the reaction layer is in fluid communication with a first reagent storage compartment at a first time and a second reagent storage compartment at a second time.

77. The fluid processing system of claim 46 wherein the diagnostic device is selected from the group comprising molecular diagnostic devices, polymerase chain reaction devices, isothermal amplification devices, lateral flow devices, devices employing arrays, electrochemical detection devices, optical detection devices, nucleic acid sequencers.

78. The fluid processing system of claim 46 further comprising an adapter configured to engage the diagnostic device and the fluid handling device.

79. The fluid processing system of claims 46 and 62 further comprising one or more device identification components.

80. The fluid processing system of claim 79 wherein the one or more device identification components are adapted and configured to identify one or more of each of serial number, manufacturer, lot number, date codes, reagent type, reagent volume, reaction area type, process identification, process parameters needed to run the process, and calibration parameter.

81. The fluid processing system of claim 80 wherein the one or more device identification components are associated with at least one or more of the diagnostic device, fluid handling device, the reagent layer, the reaction layer, and the fluid collection compartment.

82. The fluid processing system of claim 81 wherein the one or more device identification components associated with at least one or more of the fluid handling device, the reagent layer, the reaction layer, and the fluid collection compartment are adapted and configured to communicate information between the one or more of the fluid handling device, the reagent layer, the reaction layer, and the fluid collection compartment.

83. A kit for processing a sample comprising:
a reagent layer comprising,
a substrate having a first surface and an opposing second surface
at least one reagent storage compartment configured to contain a reagent, and
a seal in communication with the at least one reagent storage compartment;
a packaging adapted and configured to house one or more kit components.

84. The kit of claim 83 wherein at least one of the at least one reagent storage compartment is compressible.

85. The kit of claim 83 wherein the reagent layer further comprises two or more reagent storage compartments and further wherein at least two of the reagent storage compartments of the reagent layer are in fluid communication.

86. The kit of claim 85 wherein the two or more reagent storage compartments contain two or more reagents.

87. The kit of claim 83 wherein the reaction area further comprises one or more filters.

88. The kit of claim 83 wherein the fluid handling device further comprising at least one or more of each of a reaction layer having a first surface and an opposing second surface, comprising a reaction area, and an outlet in communication with the reaction area and a fluid collection compartment.

89. The kit of claim 83 further comprising one or more reagents.

90. The kit of claim 89 further comprising one or more syringes adapted and configured to deliver the one or more reagents to the reagent layer.

91. The kit of claim 89 wherein the reagents are selected from the group comprising:
lysis buffers, binding buffers, wash buffers, elution buffers, reaction buffers, dilution buffers, aqueous solutions, organic solutions, protein solutions, and dried reagents.

92. The kit of claim 83 further comprising a fluid collection layer.

93. The kit of claim 83 further comprising one or more of each of reaction vessels, reaction area columns, eluate collection vessels and waste compartments.

94. The kit of claim 83 further comprising an adapter adapted and configured to engage a diagnostic device and a fluid handling device.

95. The kit of claim 83 further comprising one or more detectors.

96. The kit of claim 83 further comprising one or more device identification components.

97. A kit for processing a sample comprising:
a reaction layer comprising a first surface and an opposing second surface, comprising
a reaction area, and
an outlet in communication with the reaction area;
a packaging adapted and configured to house one or more kit components.

98. The kit of claim 97 wherein the reaction area further comprises one or more filters.

99. The kit of claim 97 wherein the kit further comprises a reagent layer having a first surface and an opposing second surface, at least one reagent storage compartment configured to hold a reagent, and a seal in communication with the at least one reagent storage compartment, and a fluid collection compartment.

100. The kit of claim 98 wherein at least one of the at least one reagent storage compartment is compressible.

101. The kit of claim 98 wherein the reagent layer further comprises two or more reagent storage compartments and further wherein at least two of the reagent storage compartments of the reagent layer are in fluid communication.

102. The kit of claim 98 wherein the two or more reagent storage compartments contain two or more reagents.

103. The kit of claim 97 further comprising one or more reagents.

104. The kit of claim 97 further comprising one or more syringes adapted and configured to deliver the one or more reagents to the reagent layer.

105. The kit of claim 102 wherein the reagents are selected from the group comprising:
lysis buffers, binding buffers, wash buffers, elution buffers, reaction buffers, dilution buffers, aqueous solutions, organic solutions, protein solutions, and dried reagents.

106. The kit of claim 97 further comprising one or more of each of reaction vessels, reaction area columns, eluate collection vessels and waste compartments.

107. The kit of claim 97 further comprising an adapter adapted and configured to engage a diagnostic device and a fluid handling device.

108. The kit of claim 97 further comprising one or more detectors.

109. The kit of claim 97 further comprising one or more device identification components.

110. A communication system, comprising:
a diagnostic device;
a fluid handling device comprising an inlet for receiving a sample, a reagent layer comprising a substrate having a first surface and an opposing second surface, at least one reagent storage compartment configured to hold a reagent, and a seal in communication with the at least one reagent storage compartment, and a reaction layer having a first surface and an opposing second surface comprising, a reaction area, and an outlet in communication with the reaction area, wherein the reagent layer and the reaction layer are adapted and configured to permit movement of at least one of the reagent layer and the reaction layer in a plane relative to the other layer;
a diagnostic device server computer system;
a diagnostic test result module on the server computer system for permitting the transmission of a diagnostic test result from a diagnostic device over a network;
at least one of an API engine connected to at least one of the diagnostic device and the fluid handling device to create an message about the diagnostic test result and transmit the message over an API integrated network to a recipient having a predetermined recipient user name, an SMS engine connected to at least one of the diagnostic device and the fluid handling device to create an SMS message about the diagnostic test result and transmit the SMS message over a network to a recipient device having a predetermined diagnostic test result recipient telephone number, and an email engine connected to at least one of the diagnostic device and the fluid handling device to create an email message about the diagnostic test result and transmit the email message over the network to a diagnostic test result recipient email having a predetermined diagnostic test result recipient email address.

111. The communication system of claim 110, further comprising a storing module on the server computer system for storing the diagnostic test result on the diagnostic device server database.

112. The communications system of claim 111, wherein at least one of the diagnostic device and the fluid handling device is connectable to the server computer system over at least one of a mobile phone network and an Internet network, and a browser on the diagnostic test result recipient electronic device is used to retrieve an interface on the server computer system.

113. The communications system of claim 110, wherein a plurality of email addresses are held in a diagnostic device database and fewer than all the email addresses are individually selectable from the diagnostic host computer system, the email message being transmitted to at least one diagnostic test result recipient email having at least one selected email address.

114. The communications system of claim 113, wherein at least one of the diagnostic device and the fluid handling device is connectable to the server computer system over the Internet, and a browser on the diagnostic test result recipient electronic device is used to retrieve an interface on the server computer system.

115. The communications system of claim 110, wherein a plurality of user names are held in the diagnostic device database and fewer than all the user names are individually selectable from the diagnostic host computer system, the message being transmitted to at least one diagnostic test result recipient user name via an API.

116. The communications system of claim 115, wherein the diagnostic test result recipient electronic device is connectable to the server computer system over the Internet, and a browser on the diagnostic test result recipient electronic device is used to retrieve an interface on the server computer system.

117. The communications system of claim 110, wherein the diagnostic test result recipient electronic device is connected to the server computer system over a cellular phone network.

118. The communications system of claim 117, wherein the diagnostic test result recipient electronic device is a diagnostic test result recipient mobile device.

119. The communications system of claim 118, further comprising: an interface on the server computer system, the interface being retrievable by an application on the diagnostic test result recipient mobile device.

120. The communications system of claim 110, wherein the SMS diagnostic test result is received by a message application on the diagnostic test result recipient mobile device.

121. The communications system of claim 110, wherein a plurality of SMS diagnostic test results are received for the diagnostic test result, each by a respective message application on a respective diagnostic test result recipient mobile device.

122. The communications system of claim 110, wherein the at least one SMS engine receives an SMS response over the cellular phone SMS network from the diagnostic test result recipient mobile device and stores an SMS response on the server computer system.

123. The communications system of claim 122, wherein a diagnostic test result recipient phone number ID is transmitted with the SMS diagnostic test result to the SMS engine and is used by the server computer system to associate the SMS diagnostic test result with the SMS response.

124. The communications system of claim 110, wherein the server computer system is connectable over a cellular phone network to receive a response from the diagnostic test result recipient mobile device.

125. The communications system of claim 124, wherein the SMS diagnostic test result includes a URL that is selectable at the diagnostic test result recipient mobile device to respond from the diagnostic test result recipient mobile device to the server computer system, the server computer system utilizing the URL to associate the response with the SMS diagnostic test result.

126. The communications system of claim 110, further comprising:
a downloadable application residing on the diagnostic test result recipient mobile device, the downloadable application transmitting the response and a diagnostic test result recipient phone number ID over the cellular phone network to the server computer system, the server computer system utilizing the diagnostic test result recipient phone number ID to associate the response with the SMS diagnostic test result.

127. The communications system of claim 110, further comprising:
a transmissions module that transmits the diagnostic test result over a network other than the cellular phone SMS network to a diagnostic test result recipient user computer system, in parallel with the diagnostic test result that is sent over the cellular phone SMS network.

128. The communication system of claim 110 further comprising
a downloadable application residing on the diagnostic test result recipient host computer, the downloadable application transmitting a response and a diagnostic test result recipient phone number ID over the cellular phone network to the server computer system, the server computer system utilizing the diagnostic test result recipient phone number ID to associate the response with the SMS diagnostic test result.

129. A networked apparatus comprising:
a memory;
a processor;
a communicator;
a display;
a fluid handling device comprising an inlet for receiving a sample, a reagent layer comprising a substrate having a first surface and an opposing second surface, at least one reagent storage compartment configured to hold a reagent, and a seal in communication with the at least one reagent storage compartment, and a reaction layer having a first surface and an opposing second surface comprising, a reaction area, and an outlet in communication with the reaction area, wherein the reagent layer and the reaction layer are adapted and configured to permit movement of at least one of the reagent layer and the reaction layer in a plane relative to the other layer.
